SEQUENCE LISTING (7 sequences)

SEQ ID NO: 1 - protein, 29 aa, Homo sapiens
YADAIFTNSYRKVLGQLSARKLLQDIMSR

SEQ ID NO: 2 - protein, 29 aa, synthetic construct
  SITE 29: note = Arginine amide
YADAIFTNSYRKVLGQLSARKLLQDIMSR

SEQ ID NO: 3 - protein, 10 aa, synthetic construct
  SITE 1: note = Pyroglutamic acid
XHWSYGLRPG

SEQ ID NO: 4 - protein, 10 aa, synthetic construct
  SITE 1: note = Pyroglutamic acid
  SITE 10: note = Glycine amide
XHWSYGLRPG

SEQ ID NO: 5 - protein, 44 aa, synthetic construct
  VARIANT 2: note = Alanine or D-Alanine
  VARIANT 8: note = asparagine, D-Asparagine or alanine
  VARIANT 10: note = tyrosine or D-Tyrosine
  VARIANT 15: note = glycine, alanine or D-Alanine
  VARIANT 22: note = leucine, D-Leucine, lysine or alanine
  VARIANT 30..44: note = Any amino acid or absent
  VARIANT 30..44: note = This region may encompass 1-15 residues where the C terminal resiude is amidated
YXDAIFTXSXRKVLXQLSARKXLQDIMSRXXXXXXXXXXXXXXX

SEQ ID NO: 6 - protein, 44 aa, synthetic construct; identical to SEQ ID NO: 5
  VARIANT 2: note = alanine or D-Alanine
  VARIANT 8: note = asparagine, D-Asparagine or alanine
  VARIANT 10: note = tyrosine or D-Tyrosine
  VARIANT 15: note = glycine, alanine or D-Alanine
  VARIANT 22: note = Leucine, D-Leucine, Lysine or Alanine
  SITE 29: note = Amidated Arginine
  VARIANT 30..44: note = Any amino acid or absent
  VARIANT 30..44: note = This region may encompass 1-15 residues where the C terminal resiude is amidated

SEQ ID NO: 7 - protein, 31 aa, synthetic construct
  VARIANT 1: note = des-amino-tyrosine, acetylmethyltyrosine, or N-methyltyrosine
  VARIANT 2: note = alanine, D-Alanine, or D-alpha-aminobutanoyl
  VARIANT 6: note = phenylalanine or pentafluoro-Phe
  VARIANT 8: note = glutamine, asparagine, or threonine
  SITE 12: note = 2-chlorobenzyloxycarbonyl
  SITE 15: note = alpha-aminobutanoyl
  SITE 21: note = 2-chlorobenzyloxycarbonyl
  SITE 27: note = Norleucine
  VARIANT 28: note = Aspartic Acid or Serine
  VARIANT 29: note = Agmatine, Arginine, D-Arginine, Agmatine amide, Arginine amide, D-Arginine amide, Agmatine methylamino group, Arginine methylamino group, D-Arginine methylamino group, Agmatine ethylamino group, Arginine ethylamino group, or D-Arginine ethylamino group if this position is the C-terminus
  VARIANT 30: note = 5-aminopentanoyl, Gamma-aminobutanoyl, Glutamine, 5-aminopentanoyl amide, Gamma-aminobutanoyl amide, Glutamine amide, 5-aminopentanoyl methylamino group, Gamma-aminobutanoyl methylamino group, Glutamine methylamino group, 5-aminopentanoyl ethylamino group, Gamma-aminobutanoyl ethylamino group, Glutamine ethylamino group or absent if this position is the C-terminus
  VARIANT 31: note = Gamma-aminobutanoyl, Gamma-aminobutanoyl amide, Gamma-aminobutanoyl methylamino group, Gamma-aminobutanoyl ethylamino group, or absent
XXDAIXTXSYRXVLXQLSARXLLQDIXXXXX

(19) United States
(12) Patent Application Publication
(10) Pub. No.: US 2024/0307498 A1
(43) Pub. Date: Sep. 19, 2024

(54) GROWTH HORMONE-RELEASING HORMONE PEPTIDES AND FORMULATIONS THEREOF

(71) Applicant: ReBalance Health, Inc., Boulder, CO (US)

(72) Inventors: Russell GLENN, San Diego, CA (US); Justin HAI, Boulder, CO (US)

(21) Appl. No.: 18/681,207

(22) PCT Filed: Aug. 5, 2022
(86) PCT No.: PCT/US2022/039585

§ 371 Date: Feb. 5, 2024

Related U.S. Application Data
(60) Provisional application No. 63/230,503, filed on Aug. 6, 2021.
(60) Provisional application No. 63/234,598, filed on Aug. 18, 2021.

(51) Int. Cl. A61K 38/25; A61K 9/20; A61K 31/704; A61K 31/706; A61K 31/714; A61K 47/18; A61K 47/26

(52) U.S. Cl. CPC A61K 38/25; A61K 9/2013; A61K 31/704; A61K 31/706; A61K 31/714; A61K 47/183; A61K 47/26

(57) ABSTRACT

Described herein are sermorelin peptide compositions formulated for high oral bioavailability and high rates of absorption for treatment in a variety of applications, such as treatment of low testosterone, diminished endurance and energy, deficient immune system, slow metabolism, deficient cognitive function, poor sleep health, and poor bone health.

### PRIORITY

The application claims the benefit of U.S. Provisional Application No. 63/230,503 filed Aug. 6, 2021, and U.S. Provisional Application No. 63/234,598 filed Aug. 18, 2021, both of which are incorporated by reference herein in their entirety.

### BACKGROUND

Sermorelin peptide is a growth hormone-releasing hormone (GHRH) peptide that is useful for stimulating human growth hormone.

### SUMMARY

The present disclosure provides compositions comprising sermorelin for oral administration, e.g., in a dissolvable formulation, with high oral bioavailability and absorption rate.

In one aspect, provided herein is a composition comprising a growth hormone-releasing hormone (GHRH) peptide and glycine. In some embodiments, the glycine facilitates absorption of the peptide. In some embodiments, about 50 μg to about 3000 μg, about 50 μg to about 2900 μg, about 50 μg to about 2800 μg, about 50 μg to about 2700 μg, about 50 μg to about 2600 μg, about 50 μg to about 2500 μg, about 50 μg to about 2400 μg, about 50 μg to about 2300 μg, about 50 μg to about 2200 μg, about 50 μg to about 2100 μg, about 50 μg to about 2000 μg, about 50 μg to about 1900 μg, about 50 μg to about 1800 μg, about 50 μg to about 1700 μg, about 50 μg to about 1600 μg, about 50 μg to about 1500 μg, about 50 μg to about 1400 μg, about 50 μg to about 1300 μg, about 50 μg to about 1200 μg, about 50 μg to about 1100 μg, about 50 μg to about 1000 μg, about 50 μg to about 900 μg, about 50 μg to about 800 μg, about 50 μg to about 700 μg, about 50 μg to about 600 μg, about 50 μg to about 500 μg, about 100 μg to about 500 μg, about 200 μg to about 500 μg, about 50 μg to about 400 μg, about 50 μg to about 300 μg, about 100 μg to about 300 μg, about 200 to about 300 μg, about 100 μg to about 3000 μg, about 200 μg to about 3000 μg, about 300 μg to about 3000 μg, about 400 μg to about 3000 μg, about 500 μg to about 3000 μg, about 600 μg to about 3000 μg, about 700 μg to about 3000 μg, about 800 μg to about 3000 μg, about 900 μg to about 3000 μg, about 1000 μg to about 3000 μg, about 1100 μg to about 3000 μg, about 1200 μg to about 3000 μg, about 1300 μg to about 3000 μg, about 1400 μg to about 3000 μg, about 1500 μg to about 3000 μg, about 1600 μg to about 3000 μg, about 1700 μg to about 3000 μg, about 1800 μg to about 3000 μg, about 1900 μg to about 3000 μg, about 2000 μg to about 3000 μg, about 2100 μg to about 3000 μg, about 2200 μg to about 3000 μg, about 2300 μg to about 3000 μg, about 2400 μg to about 3000 μg, about 2500 μg to about 3000 μg, about 2600 μg to about 3000 μg, about 2700 μg to about 3000 μg, about 2800 μg to about 3000 μg, about 2900 μg to about 3000 μg, about 100 μg to about 2500 μg, about 200 μg to about 2500 μg, about 300 μg to about 2500 μg, about 400 μg to about 2500 μg, about 500 μg to about 2500 μg, about 600 μg to about 2500 μg, about 700 μg to about 2500 μg, about 800 μg to about 2500 μg, about 900 μg to about 2500 μg, about 1000 μg to about 2500 μg, about 1100 μg to about 2500 μg, about 1200 μg to about 2500 μg, about 1300 μg to about 2500 μg, about 1400 μg to about 2500 μg, about 1500 μg to about 2500 μg, about 1600 μg to about 2500 μg, about 1700 μg to about 2500 μg, about 1800 μg to about 2500 μg, about 1900 μg to about 2500 μg, about 2000 μg to about 2500 μg, about 2100 μg to about 2500 μg, about 2200 μg to about 2500 μg, about 2300 μg to about 2500 μg, about 2400 μg to about 2500 μg, about 100 μg to about 2000 μg, about 200 μg to about 2000 μg, about 300 μg to about 2000 μg, about 400 μg to about 2000 μg, about 500 μg to about 2000 μg, about 600 μg to about 2000 μg, about 700 μg to about 2000 μg, about 800 μg to about 2000 μg, about 900 μg to about 2000 μg, about 1000 μg to about 2000 μg, about 1100 μg to about 2000 μg, about 1200 μg to about 2000 μg, about 1300 μg to about 2000 μg, about 1400 μg to about 2000 μg, about 1500 μg to about 2000 μg, about 1600 μg to about 2000 μg, about 1700 μg to about 2000 μg, about 1800 μg to about 2000 μg, about 1900 μg to about 2000 μg, about 100 μg to about 1500 μg, about 200 μg to about 1500 μg, about 300 μg to about 1500 μg, about 400 μg to about 1500 μg, about 500 μg to about 1500 μg, about 600 μg to about 1500 μg, about 700 μg to about 1500 μg, about 800 μg to about 1500 μg, about 900 μg to about 1500 μg, about 1000 μg to about 1500 μg, about 1100 μg to about 1500 μg, about 1200 μg to about 1500 μg, about 1300 μg to about 1500 μg, about 1400 μg to about 1500 μg, about 100 μg to about 1000 μg, about 200 μg to about 1000 μg, about 300 μg to about 1000 μg, about 400 μg to about 1000 μg, about 500 μg to about 1000 μg, about 600 μg to about 1000 μg, about 700 μg to about 1000 μg, about 800 μg to about 1000 μg, or about 900 μg to about 1000 μg, of glycine is present in the composition. In some embodiments, about 300 μg to about 700 μg, or about 350 μg to about 700 μg, about 400 μg to about 700 μg, about 450 μg to about 700 μg, about 500 μg to about 700 μg, about 300 μg to about 650 μg, about 350 μg to about 650 μg, about 400 μg to about 650 μg, about 450 μg to about 650 μg, about 500 μg to about 650 μg, about 300 μg to about 600 μg, about 350 μg to about 600 μg, about 400 μg to about 600 μg, about 450 μg to about 600 μg, about 500 μg to about 600 μg, about 300 μg to about 550 μg, about 350 μg to about 550 μg, about 400 μg to about 550 μg, about 450 μg to about 550 μg, about 500 μg to about 550 μg, about 300 μg to about 500 μg, about 350 μg to about 500 μg, about 400 μg to about 500 μg, or about 450 μg to about 500 μg of glycine is present in the composition. In some embodiments, about 800 μg to about 1200 μg, or about 850 μg to about 1200 μg, about 900 μg to about 1200 μg, about 950 μg to about 1200 μg, about 1000 μg to about 1200 μg, about 800 μg to about 1150 μg, about 850 μg to about 1150 μg, about 900 μg to about 1150 μg, about 950 μg to about 1150 μg, about 1000 μg to about 1150 μg, about 800 μg to about 1100 μg, about 850 μg to about 1100 μg, about 900 μg to about 1100 μg, about 950 μg to about 1100 μg, about 1000 μg to about 1100 μg, about 800 μg to about 1050 μg, about 850 μg to about 1050 μg, about 900 μg to about 1050 μg, about 950 μg to about 1050 μg, about 1000 μg to about 1050 μg, about 800 μg to about 1000 μg, about 850 μg to about 1000 μg, about 900 μg to about 1000 μg, or about 950 μg to about 1000 μg of glycine is present in the composition. In some embodiments, about 1300 μg to about 1700 μg, or about 1350 μg to about 1700 μg, about 1400 μg to about 1700 μg, about 1450 μg to about 1700 μg, about 1500 μg to about 1700 μg, about 1300 μg to about 1650 μg, about 1350 μg to about 1650 μg, about 1400 μg to about 1650 μg, about 1450 μg to about 1650 μg, about 1500 μg to about 1650 μg, about 1300 μg to about 1600 μg, about 1350 μg to about 1600 μg, about 1400 μg to about 1600 μg, about 1450 μg to about 1600 μg, about 1500 μg to about 1600 μg, about 1300 μg to about 1550 μg, about 1350 μg to about 1550 μg, about 1400 μg to about 1550 μg, about 1450 μg to about 1550 μg, about 1500 μg to about 1550 μg, about 1300 μg to about 1500 μg, about 1350 μg to about 1500 μg, about 1400 μg to about 1500 μg, or about 1450 μg to about 1500 μg of glycine is present in the composition. In some embodiments, about 1800 μg to about 2200 μg, or about 1850 μg to about 2200 μg, about 1900 μg to about 2200 μg, about 1950 μg to about 2200 μg, about 2000 μg to about 2200 μg, about 1800 μg to about 2150 μg, about 1850 μg to about 2150 μg, about 1900 μg to about 2150 μg, about 1950 μg to about 2150 μg, about 2000 μg to about 2150 μg, about 1800 μg to about 2100 μg, about 1850 μg to about 2100 μg, about 1900 μg to about 2100 μg, about 1950 μg to about 2100 μg, about 2000 μg to about 2100 μg, about 1800 μg to about 2050 μg, about 1850 μg to about 2050 μg, about 1900 μg to about 2050 μg, about 1950 μg to about 2050 μg, about 2000 μg to about 2050 μg, about 1800 μg to about 2000 μg, about 1850 μg to about 2000 μg, about 1900 μg to about 2000 μg, or about 1950 μg to about 2000 μg of glycine is present in the composition. In some embodiments, about 2300 μg to about 2700 μg, or about 2350 μg to about 2700 μg, about 2400 μg to about 2700 μg, about 2450 μg to about 2700 μg, about 2500 μg to about 2700 μg, 2300 μg to about 2650 μg, about 2350 μg to about 2650 μg, about 2400 μg to about 2650 μg, about 2450 μg to about 2650 μg, about 2500 μg to about 2650 μg, about 2300 μg to about 2600 μg, about 2350 μg to about 2600 μg, about 2400 μg to about 2600 μg, about 2450 μg to about 2600 μg, about 2500 μg to about 2600 μg, about 2300 μg to about 2550 μg, about 2350 μg to about 2550 μg, about 2400 μg to about 2550 μg, about 2450 μg to about 2550 μg, about 2500 μg to about 2550 μg, 2300 μg to about 2500 μg, about 2350 μg to about 2500 μg, about 2400 μg to about 2500 μg, or about 2450 μg to about 2500 μg of glycine is present in the composition. In some embodiments, about 200 μg to about 300 μg, or about 225 μg to about 300 μg, about 250 μg to about 300 μg, about 200 to about 275 μg, about 200 to about 250 μg, or about 225 μg to about 275 μg of glycine is present in the composition. In some embodiments, about 400 ug, about 410 ug, about 420 ug, about 430 ug, about 440 ug, about 450 ug, about 460 ug, about 470 ug, about 480 ug, about 490 ug, about 500 ug, about 510 ug, about 520 ug, about 530 ug, about 540 ug, about 550 ug, about 560 ug, about 570 ug, about 580 ug, about 590 ug, or about 600 ug of glycine is present in the composition. In some embodiments, about 500 μg of glycine is present in the composition. In some embodiments, about 900 ug, about 910 ug, about 920 ug, about 930 ug, about 940 ug, about 950 ug, about 960 ug, about 970 ug, about 980 ug, about 990 ug, about 1000 ug, about 1010 ug, about 1020 ug, about 1030 ug, about 1040 ug, about 1050 ug, about 1060 ug, about 1070 ug, about 1080 ug, about 1090 ug, or about 1100 ug of glycine is present in the composition. In some embodiments, about 1000 ug of glycine is present in the composition. In some embodiments, about 1400 ug, about 1410 ug, about 1420 ug, about 1430 ug, about 1440 ug, about 1450 ug, about 1460 ug, about 1470 ug, about 1480 ug, about 1490 ug, about 1500 ug, about 1510 ug, about 1520 ug, about 1530 ug, about 1540 ug, about 1550 ug, about 1560 ug, about 1570 ug, about 1580 ug, about 1590 ug, or about 1600 ug of glycine is present in the composition. In some embodiments, about 1500 μg of glycine is present in the composition. In some embodiments, about 1900 ug, about 1910 ug, about 1920 ug, about 1930 ug, about 1940 ug, about 1950 ug, about 1960 ug, about 1970 ug, about 1980 ug, about 1990 ug, about 2000 ug, about 2010 ug, about 2020 ug, about 2030 ug, about 2040 ug, about 2050 ug, about 2060 ug, about 2070 ug, about 2080 ug, about 2090 ug, or about 2100 ug of glycine is present in the composition. In some embodiments, about 2000 μg of glycine is present in the composition. In some embodiments, about 2400 ug, about 2410 ug, about 2420 ug, about 2430 ug, about 2440 ug, about 2450 ug, about 2460 ug, about 2470 ug, about 2480 ug, about 2490 ug, about 2500 ug, about 2510 ug, about 2520 ug, about 2530 ug, about 2540 ug, about 2550 ug, about 2560 ug, about 2570 ug, about 2580 ug, about 2590 ug, or about 2600 ug of glycine is present in the composition. In some embodiments, about 2500 μg of glycine is present in the composition. In some embodiments, about 200 μg, about 210 μg, about 220 μg, about 230 μg, about 240 μg, about 250 μg, about 260 μg, about 270 μg, about 280 μg, about 290 μg, or about 300 μg of glycine is present in the composition.

In some embodiments, the composition further comprises a delivery-enhancing agent. In some embodiments, the composition further comprises a stimulatory agent. In some embodiments, the composition further comprises a steroid. In some embodiments, the composition further comprises a vitamin. In some embodiments, the composition further comprises an excipient.

In another aspect, provided herein is a composition comprising a growth hormone-releasing hormone (GHRH) peptide and a delivery-enhancing agent. In another aspect, provided herein is a composition comprising a growth hormone-releasing hormone (GHRH) peptide and a stimulatory agent. In another aspect, provided herein is a composition comprising a growth hormone-releasing hormone (GHRH) peptide and a steroid. In another aspect, provided herein is a composition comprising a growth hormone-releasing hormone (GHRH) peptide and a vitamin. In another aspect, provided herein is a composition comprising a growth hormone-releasing hormone (GHRH) peptide and an excipient.

In some embodiments, any composition herein comprises a delivery-enhancing agent. In some embodiments, the delivery-enhancing agent increases absorption of the peptide across mucous membranes. In some embodiments, the delivery-enhancing agent comprises an aggregation inhibitory agent, a charge-modifying agent, a pH control agent, a degradative enzyme inhibitory agent, a mucolytic or mucus clearing agent, a ciliostatic agent, a modulatory agent of epithelial junction physiology, a vasodilator agent, a selective transport-enhancing agent, or a membrane penetration-enhancing agent, or a combination of two or more thereof. In some embodiments, the delivery-enhancing agent comprises the membrane penetration-enhancing agent. In some embodiments, the delivery-enhancing agent comprises (i) a surfactant, (ii) a bile salt, (ii) a phospholipid additive, mixed micelle, liposome, or carrier, (iii) an alcohol, (iv) an enamine, (v) an NO donor compound, (vi) a long-chain amphipathic molecule, (vii) a small hydrophobic penetration enhancer, (viii) sodium or a salicylic acid derivative, (ix) a glycerol ester of acetoacetic acid, (x) a cyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, (x) an inhibitor of cholesterol synthesis, (xi) glycine, and (xii) any combination of the delivery-enhancing agents recited in (i)-(xi). In some embodiments, the delivery-enhancing agent comprises an alkyl glycoside. In some embodiments, the alkyl glycoside comprises a saccharide joined to a hydrophobic alkyl. In some embodiments, the saccharide comprises glucose, maltose, maltotriose, maltotetrose, sucrose or trehalose, or a combination of two or more thereof. In some embodiments, the hydrophobic alkyl has a length of about 9 to about 24 carbons. In some embodiments, the hydrophobic alkyl has a length of about 10 to about 14 carbons. In some embodiments, the alkyl glycoside comprises octyl-, nonyl-, decyl-, undecyl-, dodecyl, tridecyl, tctradecyl, pentadecyl, octadecyl a- or β-D-maltoside, -giucoside or sucroside. In some embodiments, the alkyl glycoside comprises dodecyl maltoside, tetradecyl maltoside, sucrose dodecanoate, sucrose monostearate, sucrose distearate, or a combination of two or more thereof. In some embodiments, the alkyl glycoside comprises dodecyl maltoside. In some embodiments, the alkyl glycoside comprises n-dodecyl-β-D-maltoside. In some embodiments, about 1 μg to about 30 μg, about 1 μg to about 20 μg, about 1 μg to about 15 μg, about 1 μg to about 10 μg, about 5 μg to about 30 μg, about 5 μg to about 20 μg, about 5 μg to about 15 μg, or about 5 to about 10 μg of the delivery-enhancing agent is present in the composition. In some embodiments, about 8 μg to about 15 μg, about 8 μg to about 14 μg, about 8 μg to about 13 μg, about 8 μg to about 12 μg, about 9 μg to about 15 μg, about 9 μg to about 14 μg, about 9 μg to about 13 μg, about 9 μg to about 12 μg, about 10 μg to about 15 μg, about 10 μg to about 14 μg, about 10 μg to about 13 μg, about 10 μg to about 12 μg, or about 11 μg to about 13 μg of the delivery-enhancing agent is present in the composition. In some embodiments, about 8 μg, about 9 μg, about 10 μg, about 11 μg, about 12 μg, about 13 μg, about 14 μg, or about 15 μg of the delivery-enhancing agent is present in the composition.

In some embodiments, any composition herein comprises a stimulatory agent. In some embodiments, the stimulatory agent induces secretion of human growth hormone. In some embodiments, the stimulatory agent comprises an amino acid. In some embodiments, the stimulatory agent comprises a neurotransmitter. In some embodiments, the stimulatory agent comprises glycine, choline, arginine, ornithine, glycine, lysine, glutamine, or niacin (vitamin B3), or a combination of two or more thereof. In some embodiments, the stimulatory agent comprises glycine. In some embodiments, about 50 μg to about 3000 μg, about 50 μg to about 2900 μg, about 50 μg to about 2800 μg, about 50 μg to about 2700 μg, about 50 μg to about 2600 μg, about 50 μg to about 2500 μg, about 50 μg to about 2400 μg, about 50 μg to about 2300 μg, about 50 μg to about 2200 μg, about 50 μg to about 2100 μg, about 50 μg to about 2000 μg, about 50 μg to about 1900 μg, about 50 μg to about 1800 μg, about 50 μg to about 1700 μg, about 50 μg to about 1600 μg, about 50 μg to about 1500 μg, about 50 μg to about 1400 μg, about 50 μg to about 1300 μg, about 50 μg to about 1200 μg, about 50 μg to about 1100 μg, about 50 μg to about 1000 μg, about 50 μg to about 900 μg, about 50 μg to about 800 μg, about 50 μg to about 700 μg, about 50 μg to about 600 μg, about 50 μg to about 500 μg, about 100 μg to about 500 μg, about 200 μg to about 500 μg, about 50 μg to about 400 μg, about 50 μg to about 300 μg, about 100 μg to about 300 μg, about 200 to about 300 μg, about 100 μg to about 3000 μg, about 200 μg to about 3000 μg, about 300 μg to about 3000 μg, about 400 μg to about 3000 μg, about 500 μg to about 3000 μg, about 600 μg to about 3000 μg, about 700 μg to about 3000 μg, about 800 μg to about 3000 μg, about 900 μg to about 3000 μg, about 1000 μg to about 3000 μg, about 1100 μg to about 3000 μg, about 1200 μg to about 3000 μg, about 1300 μg to about 3000 μg, about 1400 μg to about 3000 μg, about 1500 μg to about 3000 μg, about 1600 μg to about 3000 μg, about 1700 μg to about 3000 μg, about 1800 μg to about 3000 μg, about 1900 μg to about 3000 μg, about 2000 μg to about 3000 μg, about 2100 μg to about 3000 μg, about 2200 μg to about 3000 μg, about 2300 μg to about 3000 μg, about 2400 μg to about 3000 μg, about 2500 μg to about 3000 μg, about 2600 μg to about 3000 μg, about 2700 μg to about 3000 μg, about 2800 μg to about 3000 μg, about 2900 μg to about 3000 μg, about 100 μg to about 2500 μg, about 200 μg to about 2500 μg, about 300 μg to about 2500 μg, about 400 μg to about 2500 μg, about 500 μg to about 2500 μg, about 600 μg to about 2500 μg, about 700 μg to about 2500 μg, about 800 μg to about 2500 μg, about 900 μg to about 2500 μg, about 1000 μg to about 2500 μg, about 1100 μg to about 2500 μg, about 1200 μg to about 2500 μg, about 1300 μg to about 2500 μg, about 1400 μg to about 2500 μg, about 1500 μg to about 2500 μg, about 1600 μg to about 2500 μg, about 1700 μg to about 2500 μg, about 1800 μg to about 2500 μg, about 1900 μg to about 2500 μg, about 2000 μg to about 2500 μg, about 2100 μg to about 2500 μg, about 2200 μg to about 2500 μg, about 2300 μg to about 2500 μg, about 2400 μg to about 2500 μg, about 100 μg to about 2000 μg, about 200 μg to about 2000 μg, about 300 μg to about 2000 μg, about 400 μg to about 2000 μg, about 500 μg to about 2000 μg, about 600 μg to about 2000 μg, about 700 μg to about 2000 μg, about 800 μg to about 2000 μg, about 900 μg to about 2000 μg, about 1000 μg to about 2000 μg, about 1100 μg to about 2000 μg, about 1200 μg to about 2000 μg, about 1300 μg to about 2000 μg, about 1400 μg to about 2000 μg, about 1500 μg to about 2000 μg, about 1600 μg to about 2000 μg, about 1700 μg to about 2000 μg, about 1800 μg to about 2000 μg, about 1900 μg to about 2000 μg, about 100 μg to about 1500 μg, about 200 μg to about 1500 μg, about 300 μg to about 1500 μg, about 400 μg to about 1500 μg, about 500 μg to about 1500 μg, about 600 μg to about 1500 μg, about 700 μg to about 1500 μg, about 800 μg to about 1500 μg, about 900 μg to about 1500 μg, about 1000 μg to about 1500 μg, about 1100 μg to about 1500 μg, about 1200 μg to about 1500 μg, about 1300 μg to about 1500 μg, about 1400 μg to about 1500 μg, about 100 μg to about 1000 μg, about 200 μg to about 1000 μg, about 300 μg to about 1000 μg, about 400 μg to about 1000 μg, about 500 μg to about 1000 μg, about 600 μg to about 1000 μg, about 700 μg to about 1000 μg, about 800 μg to about 1000 μg, or about 900 μg to about 1000 μg, of stimulatory agent is present in the composition. In some embodiments, about 300 μg to about 700 μg, or about 350 μg to about 700 μg, about 400 μg to about 700 μg, about 450 μg to about 700 μg, about 500 μg to about 700 μg, about 300 μg to about 650 μg, about 350 μg to about 650 μg, about 400 μg to about 650 μg, about 450 μg to about 650 μg, about 500 μg to about 650 μg, about 300 μg to about 600 μg, about 350 μg to about 600 μg, about 400 μg to about 600 μg, about 450 μg to about 600 μg, about 500 μg to about 600 μg, about 300 μg to about 550 μg, about 350 μg to about 550 μg, about 400 μg to about 550 μg, about 450 μg to about 550 μg, about 500 μg to about 550 μg, about 300 μg to about 500 μg, about 350 μg to about 500 μg, about 400 μg to about 500 μg, or about 450 μg to about 500 μg of stimulatory agent is present in the composition. In some embodiments, about 800 μg to about 1200 μg, or about 850 μg to about 1200 μg, about 900 μg to about 1200 μg, about 950 μg to about 1200 μg, about 1000 μg to about 1200 μg, about 800 μg to about 1150 μg, about 850 μg to about 1150 μg, about 900 μg to about 1150 μg, about 950 μg to about 1150 μg, about 1000 μg to about 1150 μg, about 800 μg to about 1100 μg, about 850 μg to about 1100 μg, about 900 μg to about 1100 μg, about 950 μg to about 1100 μg, about 1000 μg to about 1100 μg, about 800 μg to about 1050 μg, about 850 μg to about 1050 μg, about 900 μg to about 1050 μg, about 950 μg to about 1050 μg, about 1000 μg to about 1050 μg, about 800 μg to about 1000 μg, about 850 μg to about 1000 μg, about 900 μg to about 1000 μg, or about 950 μg to about 1000 μg of stimulatory agent is present in the composition. In some embodiments, about 1300 μg to about 1700 μg, or about 1350 μg to about 1700 μg, about 1400 μg to about 1700 μg, about 1450 μg to about 1700 μg, about 1500 μg to about 1700 μg, about 1300 μg to about 1650 μg, about 1350 μg to about 1650 μg, about 1400 μg to about 1650 μg, about 1450 μg to about 1650 μg, about 1500 μg to about 1650 μg, about 1300 μg to about 1600 μg, about 1350 μg to about 1600 μg, about 1400 μg to about 1600 μg, about 1450 μg to about 1600 μg, about 1500 μg to about 1600 μg, about 1300 μg to about 1550 μg, about 1350 μg to about 1550 μg, about 1400 μg to about 1550 μg, about 1450 μg to about 1550 μg, about 1500 μg to about 1550 μg, about 1300 μg to about 1500 μg, about 1350 μg to about 1500 μg, about 1400 μg to about 1500 μg, or about 1450 μg to about 1500 μg of stimulatory agent is present in the composition. In some embodiments, about 1800 μg to about 2200 μg, or about 1850 μg to about 2200 μg, about 1900 μg to about 2200 μg, about 1950 μg to about 2200 μg, about 2000 μg to about 2200 μg, about 1800 μg to about 2150 μg, about 1850 μg to about 2150 μg, about 1900 μg to about 2150 μg, about 1950 μg to about 2150 μg, about 2000 μg to about 2150 μg, about 1800 μg to about 2100 μg, about 1850 μg to about 2100 μg, about 1900 μg to about 2100 μg, about 1950 μg to about 2100 μg, about 2000 μg to about 2100 μg, about 1800 μg to about 2050 μg, about 1850 μg to about 2050 μg, about 1900 μg to about 2050 μg, about 1950 μg to about 2050 μg, about 2000 μg to about 2050 μg, about 1800 μg to about 2000 μg, about 1850 μg to about 2000 μg, about 1900 μg to about 2000 μg, or about 1950 μg to about 2000 μg of stimulatory agent is present in the composition. In some embodiments, about 2300 μg to about 2700 μg, or about 2350 μg to about 2700 μg, about 2400 μg to about 2700 μg, about 2450 μg to about 2700 μg, about 2500 μg to about 2700 μg, 2300 μg to about 2650 μg, about 2350 μg to about 2650 μg, about 2400 μg to about 2650 μg, about 2450 μg to about 2650 μg, about 2500 μg to about 2650 μg, about 2300 μg to about 2600 μg, about 2350 μg to about 2600 μg, about 2400 μg to about 2600 μg, about 2450 μg to about 2600 μg, about 2500 μg to about 2600 μg, about 2300 μg to about 2550 μg, about 2350 μg to about 2550 μg, about 2400 μg to about 2550 μg, about 2450 μg to about 2550 μg, about 2500 μg to about 2550 μg, 2300 μg to about 2500 μg, about 2350 μg to about 2500 μg, about 2400 μg to about 2500 μg, or about 2450 μg to about 2500 μg of stimulatory agent is present in the composition. In some embodiments, about 400 ug, about 410 ug, about 420 ug, about 430 ug, about 440 ug, about 450 ug, about 460 ug, about 470 ug, about 480 ug, about 490 ug, about 500 ug, about 510 ug, about 520 ug, about 530 ug, about 540 ug, about 550 ug, about 560 ug, about 570 ug, about 580 ug, about 590 ug, or about 600 ug of stimulatory agent is present in the composition. In some embodiments, about 500 μg of stimulatory agent is present in the composition. In some embodiments, about 900 ug, about 910 ug, about 920 ug, about 930 ug, about 940 ug, about 950 ug, about 960 ug, about 970 ug, about 980 ug, about 990 ug, about 1000 ug, about 1010 ug, about 1020 ug, about 1030 ug, about 1040 ug, about 1050 ug, about 1060 ug, about 1070 ug, about 1080 ug, about 1090 ug, or about 1100 ug of stimulatory agent is present in the composition. In some embodiments, about 1000 μg of stimulatory agent is present in the composition. In some embodiments, about 1400 ug, about 1410 ug, about 1420 ug, about 1430 ug, about 1440 ug, about 1450 ug, about 1460 ug, about 1470 ug, about 1480 ug, about 1490 ug, about 1500 ug, about 1510 ug, about 1520 ug, about 1530 ug, about 1540 ug, about 1550 ug, about 1560 ug, about 1570 ug, about 1580 ug, about 1590 ug, or about 1600 ug of stimulatory agent is present in the composition. In some embodiments, about 1500 μg of stimulatory agent is present in the composition. In some embodiments, about 1900 ug, about 1910 ug, about 1920 ug, about 1930 ug, about 1940 ug, about 1950 ug, about 1960 ug, about 1970 ug, about 1980 ug, about 1990 ug, about 2000 ug, about 2010 ug, about 2020 ug, about 2030 ug, about 2040 ug, about 2050 ug, about 2060 ug, about 2070 ug, about 2080 ug, about 2090 ug, or about 2100 ug of stimulatory agent is present in the composition. In some embodiments, about 2000 μg of stimulatory agent is present in the composition. In some embodiments, about 2400 ug, about 2410 ug, about 2420 ug, about 2430 ug, about 2440 ug, about 2450 ug, about 2460 ug, about 2470 ug, about 2480 ug, about 2490 ug, about 2500 ug, about 2510 ug, about 2520 ug, about 2530 ug, about 2540 ug, about 2550 ug, about 2560 ug, about 2570 ug, about 2580 ug, about 2590 ug, or about 2600 ug of stimulatory agent is present in the composition. In some embodiments, about 2500 μg of stimulatory agent is present in the composition. In some embodiments, about 50 μg to about 500 μg, about 100 μg to about 500 μg, about 200 μg to about 500 μg, about 50 μg to about 400 μg, about 50 μg to about 300 μg, about 100 μg to about 300 μg, or about 200 to about 300 μg of the stimulatory agent is present in the composition. In some embodiments, about 200 μg to about 300 μg, or about 225 μg to about 300 μg, about 250 μg to about 300 μg, about 200 to about 275 μg, about 200 to about 250 μg, or about 225 μg to about 275 μg of the stimulatory agent is present in the composition. In some embodiments, about 200 μg, about 210 μg, about 220 μg, about 230 μg, about 240 μg, about 250 μg, about 260 μg, about 270 μg, about 280 μg, about 290 μg, or about 300 μg of the stimulatory agent is present in the composition.

In some embodiments, any composition herein comprises a steroid. In some embodiments, the steroid comprises a steroid glycoside. In some embodiments, the steroid comprises a triterpene saponin. In some embodiments, the steroid comprises a ginsenoside. In some embodiments, the ginsenoside comprises ginsenoside Rb1, ginsenoside Rb2, ginsenoside Rb3, ginsenoside Rc, ginsenoside Rd, ginsenoside Re, ginsenoside Rg1, ginsenoside Rg2, ginsenoside Rg3, or ginsenoside Rh1, or a combination of two or more thereof. In some embodiments, the ginsenoside comprises ginsenoside Rg3. In some embodiments, about 200 μg to about 2000 μg, about 500 μg to about 2000 μg, about 500 μg to about 1500 μg, about 500 μg to about 1000 μg, about 1000 μg to about 2000 μg, or about 1000 to about 1500 μg of the steroid is present in the composition. In some embodiments, about 800 μg to about 1200 μg, or about 900 μg to about 1100 μg, or about 950 μg to about 1050 μg of the steroid is present in the composition. In some embodiments, about 900 μg, about 950 μg, about 1000 μg, about 1050 μg, or about 1100 μg of the steroid is present in the composition.

In some embodiments, any composition herein comprises a vitamin. In some embodiments, the vitamin increases NAD+ levels. In some embodiments, the vitamin comprises vitamin A, vitamin C, vitamin D, vitamin E, vitamin K, vitamin B1, vitamin B2, vitamin B3, vitamin B5, vitamin B6, vitamin B7, vitamin B9, vitamin B12, or a combination of two or more thereof. In some embodiments, the vitamin comprises vitamin B3 and/or vitamin B12. In some embodiments, the vitamin comprises vitamin B3. In some embodiments, the vitamin B3 comprises nicotinamide or niacin. In some embodiments, the nicotinamide comprises nicotinamide riboside. In some embodiments, the vitamin comprises vitamin B12. In some embodiments, the vitamin B12 comprises methylcobalamin or cyanocobalamin. In some embodiments, the vitamin B12 comprises methylcobalamin. In some embodiments, about 200 μg to about 2000 μg, about 500 μg to about 2000 μg, about 500 μg to about 1500 μg, about 500 μg to about 1000 μg, about 1000 μg to about 2000 μg, or about 1000 to about 1500 μg of the vitamin is present in the composition. In some embodiments, about 800 μg to about 1200 μg, or about 900 μg to about 1100 μg, or about 950 μg to about 1050 μg of the vitamin is present in the composition. In some embodiments, about 900 μg, about 950 μg, about 1000 μg, about 1050 μg, or about 1100 μg of the vitamin is present in the composition. In some embodiments, about 400 μg to about 4000 μg, about 1000 μg to about 4000 μg, about 1000 μg to about 3000 μg, about 1000 μg to about 2000 μg, about 2000 μg to about 4000 μg, or about 2000 to about 3000 μg of the vitamin is present in the composition. In some embodiments, about 1600 μg to about 2400 μg, or about 1800 μg to about 2200 μg, or about 1900 μg to about 1100 μg of the vitamin is present in the composition. In some embodiments, about 1900 μg, about 1950 μg, about 2000 μg, about 2050 μg, or about 2100 μg of the vitamin is present in the composition.

In some embodiments, the vitamin comprises two or more vitamins. In some embodiments, the two or more vitamins comprise vitamin B3 and vitamin B12. In some embodiments, about 200 μg to about 2000 μg, about 500 μg to about 2000 μg, about 500 μg to about 1500 μg, about 500 μg to about 1000 μg, about 1000 μg to about 2000 μg, or about 1000 to about 1500 μg of each vitamin is present in the composition. In some embodiments, about 800 μg to about 1200 μg, or about 900 μg to about 1100 μg, or about 950 μg to about 1050 μg of each vitamin is present in the composition. In some embodiments, about 900 μg, about 950 μg, about 1000 μg, about 1050 μg, or about 1100 μg of each vitamin is present in the composition.

In some embodiments, any composition herein comprises an excipient. In some embodiments, the excipient is a penetration enhancer. In some embodiments, the excipient comprises an alginate. In some embodiments, the excipient comprises sodium alginate. In some embodiments, about 1 μg to about 30 μg, about 1 μg to about 20 μg, about 1 μg to about 15 μg, about 1 μg to about 10 μg, about 5 μg to about 30 μg, about 5 μg to about 20 μg, about 5 μg to about 15 μg, or about 5 to about 10 μg of the excipient is present in the composition. In some embodiments, about 8 μg to about 15 μg, about 8 μg to about 14 μg, about 8 μg to about 13 μg, about 8 μg to about 12 μg, about 9 μg to about 15 μg, about 9 μg to about 14 μg, about 9 μg to about 13 μg, about 9 μg to about 12 μg, about 10 μg to about 15 μg, about 10 μg to about 14 μg, about 10 μg to about 13 μg, about 10 μg to about 12 μg, or about 11 μg to about 13 μg of the excipient is present in the composition. In some embodiments, about 8 μg, about 9 μg, about 10 μg, about 11 μg, about 12 μg, about 13 μg, about 14 μg, or about 15 μg of the excipient is present in the composition.

In some embodiments, any composition herein comprises a GHRH peptide. In some embodiments, the GHRH peptide comprises amino acids 1-29 of human GHRH. In some embodiments, the GHRH peptide comprises a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1 (YADAIFTNSY RKVLGQLSAR KLLQDIMSR). In some embodiments, the GHRH peptide comprises a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 (YADAIFTNSY RKVLGQLSAR KLLQDIMSR-NH2). In some embodiments, the GHRH peptide comprises a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 1. In some embodiments, the GHRH peptide comprises SEQ ID NO: 1 (YADAIFTNSY RKVLGQLSAR KLLQDIMSR). In some embodiments, the GHRH peptide comprises SEQ ID NO: 2 (YADAIFTNSY RKVLGQLSAR KLLQDIMSR-NH2). In some embodiments, about 200 μg to about 2000 μg, about 500 μg to about 2000 μg, about 500 μg to about 1500 μg, about 500 μg to about 1000 μg, about 1000 μg to about 2000 μg, or about 1000 to about 1500 μg of the GHRH peptide is present in the composition. In some embodiments, about 800 μg to about 1200 μg, or about 900 μg to about 1100 μg, or about 950 μg to about 1050 μg of the GHRH peptide is present in the composition. In some embodiments, about 900 μg, about 950 μg, about 1000 μg, about 1050 μg, or about 1100 μg of the GHRH peptide is present in the composition.

In one aspect, provided herein is a formulation comprising any composition herein. In some embodiments, a composition herein is formulated in a tablet. In some embodiments, a composition herein is formulated in an oral dissolving tablet. In some embodiments, the composition disintegrates in water in less than about 30 seconds, 29 seconds, 28 seconds, 27 seconds, 26 seconds, 25 seconds, 24 seconds, 23 seconds, 22 seconds, 21 seconds, 20 seconds, 19 seconds, 18 seconds, 17 seconds, 16 seconds, 15 seconds, 14 seconds, 13 seconds, 12 seconds, 11 seconds, 10 seconds, 9 seconds, 8 seconds, 7 seconds, 6 seconds, or 5 seconds. In some embodiments, the composition disintegrates in water in less than about 10 seconds. In some embodiments, the composition is prepared in a process comprising lyophilization. In some embodiments, the composition is prepared in a unit dosage form having about 1 mg to about 10 mg weight of active ingredient. In some embodiments, the active ingredient comprises the GHRH peptide, and optionally one or more of the following: glycine, delivery-enhancing agent, stimulatory agent, steroid, vitamin, and excipient. In some embodiments, the active ingredient comprises the GHRH peptide, glycine, and optionally an excipient. In some embodiments, the unit dosage form has about 1, 2, 3, 4, 5, 6, or 7 mg of active ingredient.

In one aspect, provided herein is a method of preparing a tablet comprising any composition herein, the method comprising lyophilizing a formulation comprising the composition. In some embodiments, the formulation comprises a continuous phase. In some embodiments, the continuous phase comprises water.

In another aspect, provided herein is a method of improving the health of a subject, the method comprising administering to the subject any composition herein. In some embodiments, the administration is oral. In some embodiments, the administration is sublingual or buccal. In some embodiments, the composition increases testosterone, improves endurance, improves performance, boosts the immune system, boosts metabolism, naturally modulates hGH, improves libido, improves cognitive function, improves mood, increases energy, improves skin texture & tone, repairs DNA and maintains cell health, increases strength, helps to build lean muscles, improves muscle mass, improves skin health, improves bone health, has a calming effect, has a deep sleep effect, reduces time it takes to fall asleep, boosts daytime alertness, or any combination of two or more thereof, as compared to the subject prior to administration of the composition. In some embodiments, the composition is administered in a regimen comprises two or more doses. In some embodiments, the two or more doses is 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 doses. In some embodiments, the two or more doses is 2, 3, 4, 5, 6, 7, 8, 9, or 10 doses. In some embodiments, the two or more doses is 5 doses. In some embodiments, each dose is administered daily. In some embodiments, each dose is administered every other day, every two days, every three days, every four days, every five days, every six days, every seven days, once a week, once every two weeks, once every three weeks, or once every four weeks. In some embodiments, the regimen comprises the two or more doses, followed by one or more days where the composition is not administered to the subject. In some embodiments, the one or more days is 1, 2, 3, 4, 5, 6, or 7 days. In some embodiments, the one or more days is 2 days. In some embodiments, the regimen comprises five daily doses, followed by two days where the composition is not administered to the subject. In some embodiments, the regimen is repeated one or more times. In some embodiments, the regimen is repeated weekly, every two weeks, every three weeks, or every four weeks. In some embodiments, the regimen is repeated weekly.

The above features and methods are of representative embodiments only and are not intended to limit the scope and breadth of the claims.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 shows the structure of an exemplary delivery-enhancing agent, n-Dodecyl-β-D-maltoside.

FIG. 2 shows an example method for preparing an oral formulation for high bioavailability.

### DESCRIPTION

Provided herein are compositions and oral formulations of a composition for improving the health of a subject. In some aspects, formulations have increased oral bioavailability that provide increased efficiency for delivery of the composition. Example compositions comprise growth hormone-releasing hormone (GHRH) peptides and one or more additional agents, such as a delivery-enhancing agent and/or excipient. An additional agent may be glycine. Non-limiting example benefits of the compositions provided herein include increased testosterone, improved endurance, improved performance, a boosted immune system, a boosted metabolism, improved libido, improved cognitive function, improved mood, increased energy, improved skin texture and tone, improved muscle mass, improved skin health, improved bone health, improved sleep, improved daytime alertness, and combinations thereof.

### Growth Hormone-Releasing Hormone Peptides

In one aspect, provided are compositions and formulations comprising a growth hormone-releasing hormone (GHRH) peptide. GHRH stimulates growth hormone (GH) synthesis and secretion from the pituitary gland via binding to GHRH receptors. Growth hormone (GH) has important effects on growth and metabolism. In a nonlimiting embodiment GHRH is a chain of 29 amino acids comprising YADAIFTNSYRKVLGQLSARKLLQDIMSR (SEQ ID NO: 1). In some cases, the GHRH peptide has a c-terminal amino group such as —NH2 rather than —COOH, e.g., YADAIFTNSY RKVLGQLSAR KLLQDIMSR-NH2 (SEQ ID NO: 2).

In some embodiments, a peptide refers to a polymer of two or more amino acids. The amino acids may be joined by, e.g., peptide bonds or modified peptide bonds. The amino acids may comprise modified and/or unnatural amino acids. Modifications may be by a natural process, e.g., post-translational modifications, or by chemical modification techniques, and include disulfide bond formation, glycosylation, lipidation, acetylation, phosphorylation, or any other manipulation or modification, such as fusion with another peptide and/or conjugation. Modifications can occur anywhere in a peptide, including the peptide backbone, the amino acid side-chains, and the amino or carboxyl terminus. Modifications include, for example, acetylation, acylation, AD Pribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-link formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. Peptides may be branched or cyclic, with or without branching. Cyclic, branched and branched circular polypeptides may result from post-translational natural processes and may be made by entirely synthetic methods, as well.

In some embodiments, a peptide described herein comprises a hydrophobic amino acid. Non-limiting exemplary hydrophobic amino acids include glycine (Gly), proline (Pro), phenylalanine (Phe), alanine (Ala), isoleucine (Ile), leucine (Leu), methionine (Met), and valine (Val). In some embodiments, a hydrophobic amino acid of GHRH is replaced with another hydrophobic amino acid. In some embodiments, a peptide described herein comprises a hydrophilic amino acid. Non-limiting exemplary hydrophilic amino acids include serine (Ser), threonine (Thr), aspartic acid (Asp), glutamic acid (Glu), cysteine (Cys), asparagine (Asn), glutamine (Gln), arginine (Arg), lysine (Lys), tryptophan (Trp), tyrosine (Tyr), and histidine (His). In some embodiments, a hydrophilic amino acid of GHRH is replaced with another hydrophilic amino acid. In some embodiments, a peptide described herein comprises a basic amino acid. Non-limiting exemplary basic amino acids include arginine (Arg), lysine (Lys), and histidine (His). In some embodiments, a basic amino acid of GHRH is replaced with another basic amino acid. In some embodiments, a peptide described herein comprises an acidic amino acid. Non-limiting exemplary acidic amino acids include aspartic acid (Asp) and glutamic acid (Glu). In some embodiments, an acidic amino acid of GHRH is replaced with another acidic amino acid. In some embodiments, a peptide described herein comprises a neutral amino acid. Non-limiting exemplary neutral amino acids include alanine (Ala), asparagine (Asn), cysteine (Cys), glutamine (Gln), glycine (Gly), isoleucine (Ile), leucine (Leu), methionine (Met), phenylalanine (Phe), proline (Pro), serine (Ser), threonine (Thr), tryptophan (Trp), tyrosine (Tyr), and valine (Val). In some embodiments, a neutral amino acid of GHRH (e.g., SEQ ID NOS: 1-7) is replaced with another neutral amino acid. For example, the GHRH peptide comprising SEQ ID NO: 1 or 2 has one or more substitutions. See, for example SEQ ID NO 3-7.

Percent (%) sequence identity with respect to a reference peptide sequence is the percentage of amino acid or nucleotide residues in a candidate sequence that are identical with the amino acid residues in the reference sequence, after aligning the sequences and introducing gaps, if necessary, to achieve the maximum percent sequence identity, and not considering any conservative substitutions as part of the sequence identity. Alignment for purposes of determining percent sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, about means within 10% of the stated amount. For instance, a peptide comprising about 80% identity to a reference peptide may comprise 72% to 88% identity to the reference peptide.

### Exemplary GHRH

In certain embodiments, a GHRH peptide comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 1. The GHRH peptide may include a C-terminal amidation (e.g., —NH2). In some cases, the peptide ends in a —COOH, —NH2, —NH—CH3, or —NH—CH2CH3. In some cases, a GHRH peptide has 1, 2, 3, 4, 5, 6, 7, 8, 9, 10, 11, 12, 13, 14, or 15 amino acid substitutions as compared to a sequence of Table 1. In some cases, a GHRH peptide has 1, 2, or 3 amino acid substitutions as compared to a sequence of Table 1.

In certain embodiments, a GHRH peptide comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1. The GHRH peptide may include a C-terminal amidation (e.g., —NH2). In some cases, the peptide ends in a —COOH, —NH2, —NH—CH3, or —NH—CH2CH3. In some cases, a GHRH peptide has 1, 2, 3, 4, 5, 6, 7, 8, 9, 10, 11, 12, 13, 14, or 15 amino acid substitutions as compared to SEQ ID NO: 1. In some cases, a GHRH peptide has 1, 2, or 3 amino acid substitutions as compared to SEQ ID NO: 1. In some cases, a GHRH peptide comprises SEQ ID NO: 1.

In certain embodiments, a GHRH peptide comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2. The GHRH peptide may include a C-terminal amidation (e.g., —NH2). In some cases, the peptide ends in a —COOH, —NH2, —NH—CH3, or —NH—CH2CH3. In some cases, a GHRH peptide has 1, 2, 3, 4, 5, 6, 7, 8, 9, 10, 11, 12, 13, 14, or 15 amino acid substitutions as compared to SEQ ID NO: 2. In some cases, a GHRH peptide has 1, 2, or 3 amino acid substitutions as compared to SEQ ID NO: 2. In some cases, a GHRH peptide comprises SEQ ID NO: 2.

In certain embodiments, a GHRH peptide comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 3. The GHRH peptide may include a C-terminal amidation (e.g., —NH2). In some cases, the peptide ends in a —COOH, —NH2, —NH—CH3, or —NH—CH2CH3. In some cases, a GHRH peptide has 1, 2, 3, 4, 5, 6, 7, 8, 9, or 10 amino acid substitutions as compared to SEQ ID NO: 3. In some cases, a GHRH peptide has 1, 2, or 3 amino acid substitutions as compared to SEQ ID NO: 3. In some cases, a GHRH peptide comprises SEQ ID NO: 3.

In certain embodiments, a GHRH peptide comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 4. The GHRH peptide may include a C-terminal amidation (e.g., —NH2). In some cases, the peptide ends in a —COOH, —NH2, —NH—CH3, or —NH—CH2CH3. In some cases, a GHRH peptide has 1, 2, 3, 4, 5, 6, 7, 8, 9, or 10 amino acid substitutions as compared to SEQ ID NO: 4. In some cases, a GHRH peptide has 1, 2, or 3 amino acid substitutions as compared to SEQ ID NO: 4. In some cases, a GHRH peptide comprises SEQ ID NO: 4.

In certain embodiments, a GHRH peptide comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 5. The GHRH peptide may include a C-terminal amidation (e.g., —NH2). In some cases, the peptide ends in a —OOH, —NH2, —NH—CH3, or —NH—CH2CH3. In some cases, a GHRH peptide has 1, 2, 3, 4, 5, 6, 7, 8, 9, 10, 11, 12, 13, 14, or 15 amino acid substitutions as compared to SEQ ID NO: 5. In some cases, a GHRH peptide has 1, 2, or 3 amino acid substitutions as compared to SEQ ID NO: 5. In some cases, a GHRH peptide comprises SEQ ID NO: 5.

In certain embodiments, a GHRH peptide comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 6. The GHRH peptide may include a C-terminal amidation (e.g., —NH2). In some cases, the peptide ends in a —COOH, —NH2, —NH—CH3, or —NH—CH2CH3. In some cases, a GHRH peptide has 1, 2, 3, 4, 5, 6, 7, 8, 9, 10, 11, 12, 13, 14, or 15 amino acid substitutions as compared to SEQ ID NO: 6. In some cases, a GHRH peptide has 1, 2, or 3 amino acid substitutions as compared to SEQ ID NO: 6. In some cases, a GHRH peptide comprises SEQ ID NO: 6.

In certain embodiments, a GHRH peptide comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 7. The GHRH peptide may include a C-terminal amidation (e.g., —NH2). In some cases, the peptide ends in a —COOH, —NH2, —NH—CH3, or —NH—CH2CH3. In some cases, a GHRH peptide has 1, 2, 3, 4, 5, 6, 7, 8, 9, 10, 11, 12, 13, 14, or 15 amino acid substitutions as compared to SEQ ID NO: 7. In some cases, a GHRH peptide has 1, 2, or 3 amino acid substitutions as compared to SEQ ID NO: 7. In some cases, a GHRH peptide comprises SEQ ID NO: 7.

TABLE 1
Example GHRH sequences

| SEQ ID | | |
|---|---|---|
| NO | Description | Sequence |
| 1 | GHRHa | YADAIFTNSY RKVLGQLSAR KLLQDIMSR |
| 2 | GHRHa-NH2 | YADAIFTNSY RKVLGQLSAR KLLQDIMSR-NH2 |
| 3 | GHRHb | Pyr-HWSYGLRPG |
| 4 | GHRHb-NH2 | Pyr-HWSYGLRPG-NH2 |
| 5 | GHRHc | Y-X2-DAIFT-X8-S-X10-RKVL-X15-QLSARK-X22- |
| | | LQDIMSR-X30 |
| | | wherein X2 is Ala or D-Ala, |
| | | X8 is Asn, D-Asn or Ala, |
| | | X10 is Tyr or D-Tyr, |
| | | X15 is Gly, Ala or D-Ala, |
| | | X22 is Leu, D-Leu, Lys or Ala, and |
| | | X30 is absent, a bond or any amino acid sequence of 1 up |
| | | to 15 residues |
| | | and wherein said analogue comprises at least one of the |
| | | above amino acid substitutions in comparison with the |
| | | amino acid sequence of the native form of hGHRH1-29 |
| | | (SEQ ID NO: 1) |
| 6 | GHRHc-NH2 | Y-X2-DAIFT-X8-S-X10-RKVL-X15-QLSARK-X22- |
| | | LQDIMSR-X30-NH2 |
| | | wherein |
| | | X2 is Ala or D-Ala, |
| | | X8 is Asn, D-Asn or Ala, |
| | | X10 is Tyr or D-Tyr, |
| | | X15 is Gly, Ala or D-Ala, |
| | | X22 is Leu, D-Leu, Lys or Ala, and |
| | | X30 is absent, a bond or any amino acid sequence of 1 up |
| | | to 15 residues |
| | | and wherein said analogue comprises at least one of the |
| | | above amino acid substitutions in comparison with the |
| | | amino acid sequence of the native form of hGHRH1-29 |
| | | (SEQ ID NO: 1) |
| 7 | GHRHd | X1-X2-DAI-X6-T-X8-SYR-X12-VL-X15-QLSAR-X21- |
| | | LLQDI-X27-X28-X29-X30-X31 |
| | | wherein |
| | | X1 is des-amino-tyrosine (Dat), acetylmethyltyrosine |
| | | (Ac-Me-Tyr), or N-methyltyrosine (N-Me-Tyr), |
| | | X2 is Ala, D-Ala, or D-alpha-aminobutanoyl (D-Abu), |
| | | X6 is Phe or pentafluoro-Phe (Fpa5), |
| | | X8 is Gln, Asn, or Thr, |
| | | X12 is Orn |
| | | X15 is alpha-aminobutanoyl (Abu) |
| | | X21 is 2-chlorobenzyloxycarbonyl (Orn) |
| | | X27 is Nle |
| | | X28 is Asp or Ser |
| | | X29 is agmatine (Agm), Arg, Arg, D-Arg, or Arg, |
| | | X30 is absent, 5-aminopentanoyl(Apa), gamma- |
| | | aminobutanoyl(Gab), or Gln, and |
| | | X31 is absent or Gab, |
| | | and wherein the peptide ends in a -COOH, -NH2, -NH- |
| | | CH3, or -NH-CH2CH3 |

In some embodiments, a GHRH peptide comprises GHRHc (SEQ ID NO: 5) or GHRHc-NH2 (SEQ ID NO: 6). In some cases, X2 is D-alanine (D-Ala), X8 is alanine (Ala), X10 is tyrosine (Tyr), X15 is glycine (Gly), X22 is Lysine (Lys), and X30 is a bond. In some cases, X2 is D-alanine (D-Ala), X8 is asparagine (Asn), X10 is tyrosine (Tyr), X15 is alanine (Ala), X22 is leucine (Leu), and X30 is a bond.

In some embodiments, a GHRH peptide comprises GHRHd (SEQ ID NO: 7). In some cases, X1 is des-amino-tyrosine (Dat), X2 is alanine (Ala), X6 is phenylalanine (Phe), X8 is glutamine (Gln), X12 is 2-chlorobenzyloxycarbonyl (Orn), X15 is alpha-aminobutanoyl (Abu), X21 is Orn, X27 is norleucine (Nle), X28 is aspartate (Asp), X29 is agmatine(Agm)-NH2, and X30 and X31 are absent. In some cases, X1 is acetylmethyltyrosine (Ac-Me-Tyr), X2 is D-alanine (D-Ala), X6 is phenylalanine (Phe), X8 is glutamine (Gln), X12 is 2-chlorobenzyloxycarbonyl (Orn), X15 is alpha-aminobutanoyl (Abu), X21 is Orn, X27 is norleucine (Nle), X28 is aspartate (Asp), X29 is agmatine(Agm)-NH2, and X30 and X31 are absent. In some cases, X1 is N-methyltyrosine (N-Me-Tyr), X2 is alanine (Ala), X6 is phenylalanine (Phe), X8 is glutamine (Gln), X12 is 2-chlorobenzyloxycarbonyl (Or), X15 is alpha-aminobutanoyl (Abu), X21 is Orn, X27 is norleucine (Nle), X28 is aspartate (Asp), X29 is agmatine(Agm)-NH2, and X30 and X31 are absent. In some cases, X1 is N-methyltyrosine (N-Me-Tyr), X2 is D-alanine (D-Ala), X6 is phenylalanine (Phe), X8 is glutamine (Gln), X12 is 2-chlorobenzyloxycarbonyl (Orn), X15 is alpha-aminobutanoyl (Abu), X21 is Orn, X27 is norleucine (Nle), X28 is aspartate (Asp), X29 is agmatine(Agm)-NH2, and X30 and X31 are absent. In some cases, X1 is N-methyltyrosine (N-Me-Tyr), X2 is D-alanine (D-Ala), X6 is phenylalanine (Phe), X8 is asparagine (Asn), X12 is 2-chlorobenzyloxycarbonyl (Orn), X15 is alpha-aminobutanoyl (Abu), X21 is Orn, X27 is norleucine (Nle), X28 is aspartate (Asp), X29 is agmatine(Agm)-NH2, and X30 and X31 are absent. In some cases, X1 is des-amino-tyrosine (Dat), X2 is D-alanine (D-Ala), X6 is phenylalanine (Phe), X8 is glutamine (Gln), X12 is 2-chlorobenzyloxycarbonyl (Orn), X15 is alpha-aminobutanoyl (Abu), X21 is Orn, X27 is norleucine (Nle), X28 is aspartate (Asp), X29 is D-arginine(D-Arg)-NH-CH3, and X30 and X31 are absent. In some cases, X1 is N-methyltyrosine (N-Me-Tyr), X2 is D-alanine (D-Ala), X6 is phenylalanine (Phe), X8 is glutamine (Gln), X12 is 2-chlorobenzyloxycarbonyl (Orn), X15 is alpha-aminobutanoyl (Abu), X21 is Orn, X27 is norleucine (Nle), X28 is aspartate (Asp), X29 is arginine(Arg)-NH—CH3, and X30 and X31 are absent. In some cases, X1 is N-methyltyrosine (N-Me-Tyr), X2 is D-alanine (D-Ala), X6 is pentafluoro-Phe (Fpa5), X8 is glutamine (Gln), X12 is 2-chlorobenzyloxycarbonyl (Orn), X15 is alpha-aminobutanoyl (Abu), X21 is Orn, X27 is norleucine (Nle), X28 is aspartate (Asp), X29 is arginine(Arg)-NH—CH3, and X30 and X31 are absent. In some cases, X1 is N-methyltyrosine (N-Me-Tyr), X2 is alanine (Ala), X6 is phenylalanine (Phe), X8 is glutamine (Gln), X12 is 2-chlorobenzyloxycarbonyl (Orn), X15 is alpha-aminobutanoyl (Abu), X21 is Orn, X27 is norleucine (Nle), X28 is serine (Ser), X29 is arginine(Arg)-NH—CH3, and X30 and X31 are absent. In some cases, X1 is N-methyltyrosine (N-Me-Tyr), X2 is alanine (Ala), X6 is phenylalanine (Phe), X8 is glutamine (Gln), X12 is 2-chlorobenzyloxycarbonyl (Orn), X15 is alpha-aminobutanoyl (Abu), X21 is Orn, X27 is norleucine (Nle), X28 is aspartate (Asp), X29 is arginine(Arg)-NH—CH3, and X30 and X31 are absent. In some cases, X1 is des-amino-tyrosine (Dat), X2 is alanine (Ala), X6 is phenylalanine (Phe), X8 is glutamine (Gln), X12 is 2-chlorobenzyloxycarbonyl (Orn), X15 is alpha-aminobutanoyl (Abu), X21 is Orn, X27 is norleucine (Nle), X28 is aspartate (Asp), X29 is arginine(Arg)-NH-CH3, and X30 and X31 are absent. In some cases, X1 is des-amino-tyrosine (Dat), X2 is D-alanine (D-Ala), X6 is phenylalanine (Phe), X8 is glutamine (Gln), X12 is 2-chlorobenzyloxycarbonyl (Orn), X15 is alpha-aminobutanoyl (Abu), X21 is Orn, X27 is norleucine (Nle), X28 is aspartate (Asp), X29 is arginine(Arg)-NH—CH3, and X30 and X31 are absent. In some cases, X1 is N-methyltyrosine (N-Me-Tyr), X2 is D-alanine (D-Ala), X6 is phenylalanine (Phe), X8 is asparagine (Asn), X12 is 2-chlorobenzyloxycarbonyl (Orn), X15 is alpha-aminobutanoyl (Abu), X21 is Orn, X27 is norleucine (Nle), X28 is aspartate (Asp), X29 is arginine(Arg)-NH—CH3, and X30 and X31 are absent. In some cases, X1 is N-methyltyrosine (N-Me-Tyr), X2 is D-alanine (D-Ala), X6 is phenylalanine (Phe), X8 is threonine (Thr), X12 is 2-chlorobenzyloxycarbonyl (Or), X15 is alpha-aminobutanoyl (Abu), X21 is Orn, X27 is norleucine (Nle), X28 is aspartate (Asp), X29 is arginine(Arg)-NH—CH3, and X30 and X31 are absent. In some cases, X1 is N-methyltyrosine (N-Me-Tyr), X2 is D-alanine (D-Ala), X6 is phenylalanine (Phe), X8 is glutamine (Gln), X12 is 2-chlorobenzyloxycarbonyl (Orn), X15 is alpha-aminobutanoyl (Abu), X21 is Orn, X27 is norleucine (Nle), X28 is aspartate (Asp), X29 is arginine(Arg)-NH—CH2CH3, and X30 and X31 are absent. In some cases, X1 is N-methyltyrosine (N-Me-Tyr), X2 is D-alanine (D-Ala), X6 is phenylalanine (Phe), X8 is asparagine (Asn), X12 is 2-chlorobenzyloxycarbonyl (Orn), X15 is alpha-aminobutanoyl (Abu), X21 is Orn, X27 is norleucine (Nle), X28 is aspartate (Asp), X29 is arginine(Arg)-NH—CH2CH3, and X30 and X31 are absent. In some cases, X1 is N-methyl-tyrosine (N-Me-Tyr), X2 is D-alanine (D-Ala), X6 is phenylalanine (Phe), X8 is glutamine (Gln), X12 is 2-chlorobenzyloxycarbonyl (Orn), X15 is alpha-aminobutanoyl (Abu), X21 is Orn, X27 is norleucine (Nle), X28 is aspartate (Asp), X29 is arginine (Arg), X30 is 5-aminopentanoyl-NH2 (Apa-NH2) and X31 is absent. In some cases, X1 is des-amino-tyrosine (Dat), X2 is alanine (Ala), X6 is phenylalanine (Phe), X8 is glutamine (Gln), X12 is 2-chlorobenzyloxycarbonyl (Orn), X15 is alpha-aminobutanoyl (Abu), X21 is Orn, X27 is norleucine (Nle), X28 is aspartate (Asp), X29 is arginine (Arg), X30 is 5-aminopentanoyl-NH2 (Apa-NH2) and X31 is absent. In some cases, X1 is des-amino-tyrosine (Dat), X2 is D-alanine (D-Ala), X6 is pentafluoro-Phe (Fpa5), X8 is asparagine (Asn), X12 is 2-chlorobenzyloxycarbonyl (Orn), X15 is alpha-aminobutanoyl (Abu), X21 is Orn, X27 is norleucine (Nle), X28 is serine (Ser), X29 is arginine (Arg), X30 is gamma-aminobutanoyl-NH2 (Gab-NH2) and X31 is absent. In some cases, X1 is des-amino-tyrosine (Dat), X2 is D-alpha-aminobutanoyl (D-Abu), X6 is pentafluoro-Phe (Fpa5), X8 is asparagine (Asn), X12 is 2-chlorobenzyloxycarbonyl (Orn), X15 is alpha-aminobutanoyl (Abu), X21 is Orn, X27 is norleucine (Nle), X28 is serine (Ser), X29 is arginine (Arg), X30 is gamma-aminobutanoyl-NH2 (Gab-NH2) and X31 is absent. In some cases, X1 is des-amino-tyrosine (Dat), X2 is D-alanine (D-Ala), X6 is pentafluoro-Phe (Fpa5), X8 is asparagine (Asn), X12 is 2-chlorobenzyloxycarbonyl (Orn), X15 is alpha-aminobutanoyl (Abu), X21 is Orn, X27 is norleucine (Nle), X28 is serine (Ser), X29 is arginine (Arg), X30 is glutamine (Gln) and X31 is gamma-aminobutanoyl-NH2 (Gab-NH2).

GHRHb is a small 10 amino acid long peptide. It acts as the hypophysis to cause an increase in the release of biologically active Follicle-Stimulating Hormone and Luteinizing Hormone in the blood. In turn, these hormones stimulate the development of male and female steroid in growing male and female animals.

GHRHc is a 29 amino acid peptide. As an analog of GHRH, GHRHc stimulates production of growth hormone in a mammal. There are permutations of the analog described above in Table 1.

GHRHd is a 29-31 amino acid peptide. GHRHd acts as an analog to GHRH stimulating production of growth hormone in a mammal. There are permutations of the analog described above in Table 1.

### Methods of Generating Peptides

Peptides may be prepared using methods known in the art. At its core, peptide synthesis is the stepwise assembly of peptides from amino acid precursors. Non-limiting examples of peptide synthesis methods include in vivo and in vitro translation systems, and organic synthesis routes such as solid phase peptide synthesis. Solid phase peptide synthesis is a technique wherein an initial amino acid is linked to a solid surface. Non-limiting examples of solid surfaces include a bead, a microscope slide, or a similar surface. Following this, subsequent amino acids are added to the initial amino acid to form the peptide. The peptide is attached to a solid surface, so operations such as wash steps, side chain modifications, cyclization, or other treatment steps can be performed with the peptide maintained in a discrete location. Additional methods include use of automated synthesis platforms for parallel assembly of millions of unique peptides in an array on a single surface. Chemical peptide synthesis methods facilitate facile synthesis of peptide analogs such as the ones provided herein. Synthetic polypeptides can be synthesized, for example, using an automated polypeptide synthesizer.

Peptides may be synthesized by standard solid-phase peptide synthesis (SPPS) techniques and purified via HPLC. Peptides may be synthesized using standard Fmoc chemistry. Peptide purification by flash chromatography may be performed on silica gel prepacked columns (40 μm, RediSep® Rf from Teledyne Isco) on a CombiFlash® Rf (Teledyne Isco).

### Compositions

In one aspect, provided herein are compositions comprising a GHRH peptide (e.g., as described herein) that are useful in a variety of applications including, but not limited to, therapeutic applications, such as the treatment of low testosterone, diminished endurance and energy, deficient immune system, slow metabolism, deficient cognitive function, poor sleep health, and bone health issues.

In various embodiments, the compositions are formulated for delivery via any route of administration. A route of administration includes any administration pathway known in the art, including but not limited to intravenous, subcutaneous, aerosol, nasal, oral (sublingual or buccal), transmucosal, transdermal and parenteral. In example embodiments, the route of administration is oral (e.g., buccal, sublingual), dermal, or intranasal.

In various aspects, the composition is formulated for buccal or sublingual delivery. Buccal or sublingual delivery can provide a means for rapid transmucosal absorption of a portion of the peptide in the presence of one or more excipients, absorption enhancing, and/or delivery-enhancing agents. Delivery-enhancing agents include mucosal delivery-enhancing agents. In some aspects, the composition comprises the GHRH peptide, glycine, and optionally one or more excipients, absorption enhancing, and/or delivery-enhancing agents. In some cases, glycine may function as an excipient, absorption enhancing agent, or delivery-enhancing agent, or a combination thereof.

In non-limiting embodiments, a delivery-enhancing agent comprises an alkyl glycoside (or alkyl saccharide). In certain aspects, upon administration of peptide composition by placing it in contact with buccal tissue, addition of an alkyl saccharide to the composition results in substantially reduced first-pass effect metabolism of the peptide composition. Administration of fast dispersing formulations upon presentation to buccal tissue results in an increase in bioavailability. Additional aspects of the compositions and formulations herein are based on the notion that increasing or decreasing the amount of specific alkyl saccharides included in fast-dispersing dosage forms alters or modulates the site of absorption of a peptide compound, increasing or decreasing, respectively, that proportion of a peptide that is absorbed through buccal tissue compared to other portions of the alimentary canal. In cases where it is desirable to speed the onset of peptide action but preserve the normally longer Tmax associated with the standard oral tablet, the alkyl saccharide content can be reduced to attenuate buccal absorption so that a portion of the drug is immediately absorbed buccally for rapid onset, but the rest is absorbed through the slower gastric absorption process.

Further non-limiting examples of mucosal delivery-enhancing agents include: cyclopentadecalactone; sodium N-[8-(2-hydroxylbenzoyl)amino] caprylate (SNAC); medium chain fatty acids, salts, and derivatives; sodium caprate, modified release formulation; sodium caprylate suspension in hydrophobic medium with matrix forming polymer; protease inhibitor and omega 3 fatty acid; liquid mixed-micelle spray; combo of protease inhibitor, permeation enhancer, pH modifier, enteric coating; lipid polymer micelle; chitosan; dodecyl-2-N,N-dimethylamino propionate (DDAIP), and other agents known in the art. Other mucosal delivery-enhancing agents include glycine, aggregation inhibitory agents; charge-modifying agents; pH control agents; degradative enzyme inhibitory agents; mucolytic or mucus clearing agents; ciliostatic agents; membrane penetration-enhancing agents; modulatory agents of epithelial junction physiology; vasodilator agents; selective transport-enhancing agents; and stabilizing delivery vehicles, carriers, mucoadhesives, support or complex-forming species. Additional mucosal delivery-enhancing agents include, for example, citric acid, sodium citrate, propylene glycol, glycerin, ascorbic acid (e.g., L-ascorbic acid), sodium metabisulfite, ethylenediaminetetraacetic acid (EDTA) disodium, benzalkonium chloride, sodium hydroxide, and mixtures thereof. In some cases, delivery enhancing agent comprises a membrane penetration enhancing agents, such as a surfactant; a bile salt; a phospholipid additive, mixed micelle, liposome, or carrier; an alcohol; an enamine; an NO donor compound; a long-chain amphipathic molecule; a small hydrophobic penetration enhancer; sodium or a salicylic acid derivative; a glycerol ester of acetoacetic acid; a cyclodextrin or beta-cyclodextrin derivative; a medium-chain fatty acid; a chelating agent; an amino acid or salt thereof; an N-acetylamino acid or salt thereof; an enzyme degradative to a selected membrane component; an inhibitor of fatty acid synthesis; an inhibitor of cholesterol synthesis; and any combination of the membrane penetration enhancing agents recited above.

In some embodiments, amino acids act as delivery enhancing agents. In a nonlimiting example embodiment, glycine is used as a delivery enhancing agent.

Mucosal delivery-enhancing agents include agents which enhance the release or solubility (e.g., from a formulation delivery vehicle), diffusion rate, penetration capacity and timing, uptake, residence time, stability, effective half-life, peak or sustained concentration levels, clearance and other desired mucosal delivery characteristics (e.g., as measured at the site of delivery, or at a selected target site of activity such as the bloodstream or central nervous system) of a peptide. Enhancement of mucosal delivery can occur a variety of mechanisms, including, for example, by increasing the diffusion, transport, persistence or stability of the compound, increasing membrane fluidity, modulating the availability or action of calcium and other ions that regulate intracellular or paracellular permeation, solubilizing mucosal membrane components (e.g., lipids), changing non-protein and protein sulfhydryl levels in mucosal tissues, increasing water flux across the mucosal surface, modulating epithelial junction physiology, reducing the viscosity of mucus overlying the mucosal epithelium, reducing mucociliary clearance rates, and other mechanisms.

In some cases, compositions herein are formulated in an oral-dispersing dosage form, e.g., a fast-dispersing dosage form, which encompasses all the types of dosage forms capable of dissolving, entirely or in part, within the mouth. However, in certain aspects, the oral-dispersing dosage form is a solid, fast-dispersing network of peptide and a water-soluble or water-dispersible carrier matrix which is inert towards the peptide and excipients. In some embodiments, the network may be obtained by lyophilizing or subliming solvent from a composition in the solid state, which composition comprises the peptide, an alkyl saccharide, and a solution of the carrier in a solvent. While a variety of solvents are known in the art as being suitable for this use, one solvent particularly well suited for use herein is water. Water-alcohol mixtures may also be employed where drug solubility in the mixed solvent is enhanced. For poorly water-soluble drugs, dispersions of small drug particles can be suspended in an aqueous gel that maintains uniform distribution of the substantially insoluble drug during the lyophilization or subliming process. FIG. 2 provides an example preparation of an oral-dispersing dosage form.

### Non-Limiting Excipients

In various embodiments, compositions herein including an acceptable excipient along with the active ingredient (e.g., peptide, stimulatory agent, steroid, vitamin). An acceptable excipient may be an excipient that is useful in preparing a composition that is generally safe, non-toxic, and desirable, and includes excipients that are acceptable for veterinary use as well as for human use. The active ingredient can be mixed with excipients which are acceptable and compatible with the active ingredient and in amounts suitable for use in therapeutic methods described herein. Such excipients may be solid, liquid, semisolid, or, in the case of an aerosol composition, gaseous. Suitable excipients are, for example, starch, glucose, lactose, sucrose, gelatin, malt, rice, flour, chalk, silica gel, sodium stearate, glycerol monostearate, talc, sodium chloride, dried skim milk, water, saline, dextrose, propylene glycol, glycerol, ethanol, mannitol, polysorbate or the like and combinations thereof. In addition, if desired, the composition can contain auxiliary substances such as wetting or emulsifying agents, pH buffering agents and the like which enhance or maintain the effectiveness of the active ingredient. In some embodiments acceptable excipients, or enhancing agents, can be used for effective delivery of the composition. Excipients may act as stabilizing agents in compositions and formulations of the present disclosure. Therapeutic compositions as described herein can include acceptable salts. Acceptable salts include the acid addition salts formed with inorganic acids such as, for example, hydrochloric or phosphoric acids, organic acids, for example, acetic, tartaric or mandelic, salts formed from inorganic bases such as, for example, sodium, potassium, ammonium, calcium or ferric hydroxides, and salts formed from organic bases such as isopropylamine, trimethylamine, 2-ethylamino ethanol, histidine, procaine and the like. Liquid compositions can contain liquid phases in addition to and in the exclusion of water, for example, glycerin, vegetable oils such as cottonseed oil, and water-oil emulsions. Physiologically tolerable carriers are well known in the art.

In various embodiments, compositions herein including an acceptable carrier along with the active ingredient (e.g., peptide, stimulatory agent, steroid, vitamin). In some cases, an acceptable carrier refers to an acceptable material, composition, or vehicle that is involved in carrying or transporting the ingredient from one tissue, organ, or portion of the body to another tissue, organ, or portion of the body. For example, the carrier may be a liquid or solid filler, diluent, excipient, solvent, or encapsulating material, or a combination thereof. Each component of the carrier must be acceptable in that it must be compatible with the other ingredients of the formulation. It must also be suitable for use in contact with any tissues or organs with which it may come in contact, meaning that it does not carry a risk of toxicity, irritation, allergic response, immunogenicity, or any other complication that excessively outweighs its therapeutic benefits.

The amount of active ingredient (e.g., peptide, stimulatory agent, steroid, vitamin) used that will be effective in the therapeutic methods of some embodiments will depend on the nature of the disorder or condition and can be determined by one of skill in the art with standard techniques.

### Non-Limiting Example Compositions

In certain embodiments, a GHRH peptide is formulated in a composition with one or more agents. The one or more agents may be active or inactive agents, where an active agent is present in the composition for a beneficial effect in the subject, e.g., increased testosterone, improved endurance, improved performance, a boosted immune system, a boosted metabolism, improved libido, improved cognitive function, improved mood, increased energy, improved skin texture and tone, improved muscle mass, improved skin health, improved bone health, improved sleep, improved daytime alertness, and combinations thereof. Non-limiting examples of agents include delivery-enhancing agents, surfactants, alkyl glycosides, stimulatory agents, neurotransmitters, amino acids, steroids, vitamins, and excipients.

In some embodiments, about 200 μg to about 2000 μg, about 500 μg to about 2000 μg, about 500 μg to about 1500 μg, about 500 μg to about 1000 μg, about 1000 μg to about 2000 μg, or about 1000 to about 1500 μg of the GHRH peptide is present in the composition. In some cases, about 800 μg to about 1200 μg, or about 900 μg to about 1100 μg, or about 950 μg to about 1050 μg of the GHRH peptide is present in the composition. In some cases, about 900 μg, about 950 μg, about 1000 μg, about 1050 μg, or about 1100 μg of the GHRH peptide is present in the composition.

In certain embodiments, a composition comprising a GHRH peptide comprises glycine. In some embodiments, about 50 μg to about 3000 μg, about 50 μg to about 2900 μg, about 50 μg to about 2800 μg, about 50 μg to about 2700 μg, about 50 μg to about 2600 μg, about 50 μg to about 2500 μg, about 50 μg to about 2400 μg, about 50 μg to about 2300 μg, about 50 μg to about 2200 μg, about 50 μg to about 2100 μg, about 50 μg to about 2000 μg, about 50 μg to about 1900 μg, about 50 μg to about 1800 μg, about 50 μg to about 1700 μg, about 50 μg to about 1600 μg, about 50 μg to about 1500 μg, about 50 μg to about 1400 μg, about 50 μg to about 1300 μg, about 50 μg to about 1200 μg, about 50 μg to about 1100 μg, about 50 μg to about 1000 μg, about 50 μg to about 900 μg, about 50 μg to about 800 μg, about 50 μg to about 700 μg, about 50 μg to about 600 μg, about 50 μg to about 500 μg, about 100 μg to about 500 μg, about 200 μg to about 500 μg, about 50 μg to about 400 μg, about 50 μg to about 300 μg, about 100 μg to about 300 μg, about 200 to about 300 μg, about 100 μg to about 3000 μg, about 200 μg to about 3000 μg, about 300 μg to about 3000 μg, about 400 μg to about 3000 μg, about 500 μg to about 3000 μg, about 600 μg to about 3000 μg, about 700 μg to about 3000 μg, about 800 μg to about 3000 μg, about 900 μg to about 3000 μg, about 1000 μg to about 3000 μg, about 1100 μg to about 3000 μg, about 1200 μg to about 3000 μg, about 1300 μg to about 3000 μg, about 1400 μg to about 3000 μg, about 1500 μg to about 3000 μg, about 1600 μg to about 3000 μg, about 1700 μg to about 3000 μg, about 1800 μg to about 3000 μg, about 1900 μg to about 3000 μg, about 2000 μg to about 3000 μg, about 2100 μg to about 3000 μg, about 2200 μg to about 3000 μg, about 2300 μg to about 3000 μg, about 2400 μg to about 3000 μg, about 2500 μg to about 3000 μg, about 2600 μg to about 3000 μg, about 2700 μg to about 3000 μg, about 2800 μg to about 3000 μg, about 2900 μg to about 3000 μg, about 100 μg to about 2500 μg, about 200 μg to about 2500 μg, about 300 μg to about 2500 μg, about 400 μg to about 2500 μg, about 500 μg to about 2500 μg, about 600 μg to about 2500 μg, about 700 μg to about 2500 μg, about 800 μg to about 2500 μg, about 900 μg to about 2500 μg, about 1000 μg to about 2500 μg, about 1100 μg to about 2500 μg, about 1200 μg to about 2500 μg, about 1300 μg to about 2500 μg, about 1400 μg to about 2500 μg, about 1500 μg to about 2500 μg, about 1600 μg to about 2500 μg, about 1700 μg to about 2500 μg, about 1800 μg to about 2500 μg, about 1900 μg to about 2500 μg, about 2000 μg to about 2500 μg, about 2100 μg to about 2500 μg, about 2200 μg to about 2500 μg, about 2300 μg to about 2500 μg, about 2400 μg to about 2500 μg, about 100 μg to about 2000 μg, about 200 μg to about 2000 μg, about 300 μg to about 2000 μg, about 400 μg to about 2000 μg, about 500 μg to about 2000 μg, about 600 μg to about 2000 μg, about 700 μg to about 2000 μg, about 800 μg to about 2000 μg, about 900 μg to about 2000 μg, about 1000 μg to about 2000 μg, about 1100 μg to about 2000 μg, about 1200 μg to about 2000 μg, about 1300 μg to about 2000 μg, about 1400 μg to about 2000 μg, about 1500 μg to about 2000 μg, about 1600 μg to about 2000 μg, about 1700 μg to about 2000 μg, about 1800 μg to about 2000 μg, about 1900 μg to about 2000 μg, about 100 μg to about 1500 μg, about 200 μg to about 1500 μg, about 300 μg to about 1500 μg, about 400 μg to about 1500 μg, about 500 μg to about 1500 μg, about 600 μg to about 1500 μg, about 700 μg to about 1500 μg, about 800 μg to about 1500 μg, about 900 μg to about 1500 μg, about 1000 μg to about 1500 μg, about 1100 μg to about 1500 μg, about 1200 μg to about 1500 μg, about 1300 μg to about 1500 μg, about 1400 μg to about 1500 μg, about 100 μg to about 1000 μg, about 200 μg to about 1000 μg, about 300 μg to about 1000 μg, about 400 μg to about 1000 μg, about 500 μg to about 1000 μg, about 600 μg to about 1000 μg, about 700 μg to about 1000 μg, about 800 μg to about 1000 μg, or about 900 μg to about 1000 μg of glycine is present in the composition. In some embodiments, about 300 μg to about 700 μg, or about 350 μg to about 700 μg, about 400 μg to about 700 μg, about 450 μg to about 700 μg, about 500 μg to about 700 μg, about 300 μg to about 650 μg, about 350 μg to about 650 μg, about 400 μg to about 650 μg, about 450 μg to about 650 μg, about 500 μg to about 650 μg, about 300 μg to about 600 μg, about 350 μg to about 600 μg, about 400 μg to about 600 μg, about 450 μg to about 600 μg, about 500 μg to about 600 μg, about 300 μg to about 550 μg, about 350 μg to about 550 μg, about 400 μg to about 550 μg, about 450 μg to about 550 μg, about 500 μg to about 550 μg, about 300 μg to about 500 μg, about 350 μg to about 500 μg, about 400 μg to about 500 μg, or about 450 μg to about 500 μg of glycine is present in the composition. In some embodiments, about 800 μg to about 1200 μg, or about 850 μg to about 1200 μg, about 900 μg to about 1200 μg, about 950 μg to about 1200 μg, about 1000 μg to about 1200 μg, about 800 μg to about 1150 μg, about 850 μg to about 1150 μg, about 900 μg to about 1150 μg, about 950 μg to about 1150 μg, about 1000 μg to about 1150 μg, about 800 μg to about 1100 μg, about 850 μg to about 1100 μg, about 900 μg to about 1100 μg, about 950 μg to about 1100 μg, about 1000 μg to about 1100 μg, about 800 μg to about 1050 μg, about 850 μg to about 1050 μg, about 900 μg to about 1050 μg, about 950 μg to about 1050 μg, about 1000 μg to about 1050 μg, about 800 μg to about 1000 μg, about 850 μg to about 1000 μg, about 900 μg to about 1000 μg, or about 950 μg to about 1000 μg of glycine is present in the composition. In some embodiments, about 1300 μg to about 1700 μg, or about 1350 μg to about 1700 μg, about 1400 μg to about 1700 μg, about 1450 μg to about 1700 μg, about 1500 μg to about 1700 μg, about 1300 μg to about 1650 μg, about 1350 μg to about 1650 μg, about 1400 μg to about 1650 μg, about 1450 μg to about 1650 μg, about 1500 μg to about 1650 μg, about 1300 μg to about 1600 μg, about 1350 μg to about 1600 μg, about 1400 μg to about 1600 μg, about 1450 μg to about 1600 μg, about 1500 μg to about 1600 μg, about 1300 μg to about 1550 μg, about 1350 μg to about 1550 μg, about 1400 μg to about 1550 μg, about 1450 μg to about 1550 μg, about 1500 μg to about 1550 μg, about 1300 μg to about 1500 μg, about 1350 μg to about 1500 μg, about 1400 μg to about 1500 μg, or about 1450 μg to about 1500 μg of glycine is present in the composition. In some embodiments, about 1800 μg to about 2200 μg, or about 1850 μg to about 2200 μg, about 1900 μg to about 2200 μg, about 1950 μg to about 2200 μg, about 2000 μg to about 2200 μg, about 1800 μg to about 2150 μg, about 1850 μg to about 2150 μg, about 1900 μg to about 2150 μg, about 1950 μg to about 2150 μg, about 2000 μg to about 2150 μg, about 1800 μg to about 2100 μg, about 1850 μg to about 2100 μg, about 1900 μg to about 2100 μg, about 1950 μg to about 2100 μg, about 2000 μg to about 2100 μg, about 1800 μg to about 2050 μg, about 1850 μg to about 2050 μg, about 1900 μg to about 2050 μg, about 1950 μg to about 2050 μg, about 2000 μg to about 2050 μg, about 1800 μg to about 2000 μg, about 1850 μg to about 2000 μg, about 1900 μg to about 2000 μg, or about 1950 μg to about 2000 μg of glycine is present in the composition. In some embodiments, about 2300 μg to about 2700 μg, or about 2350 μg to about 2700 μg, about 2400 μg to about 2700 μg, about 2450 μg to about 2700 μg, about 2500 μg to about 2700 μg, 2300 μg to about 2650 μg, about 2350 μg to about 2650 μg, about 2400 μg to about 2650 μg, about 2450 μg to about 2650 μg, about 2500 μg to about 2650 μg, about 2300 μg to about 2600 μg, about 2350 μg to about 2600 μg, about 2400 μg to about 2600 μg, about 2450 μg to about 2600 μg, about 2500 μg to about 2600 μg, about 2300 μg to about 2550 μg, about 2350 μg to about 2550 μg, about 2400 μg to about 2550 μg, about 2450 μg to about 2550 μg, about 2500 μg to about 2550 μg, 2300 μg to about 2500 μg, about 2350 μg to about 2500 μg, about 2400 μg to about 2500 μg, or about 2450 μg to about 2500 μg of glycine is present in the composition. In some cases, about 200 μg to about 300 μg, or about 225 μg to about 300 μg, about 250 μg to about 300 μg, about 200 to about 275 μg, about 200 to about 250 μg, or about 225 μg to about 275 μg of glycine is present in the composition. In some cases, about 400 ug, about 410 ug, about 420 ug, about 430 ug, about 440 ug, about 450 ug, about 460 ug, about 470 ug, about 480 ug, about 490 ug, about 500 ug, about 510 ug, about 520 ug, about 530 ug, about 540 ug, about 550 ug, about 560 ug, about 570 ug, about 580 ug, about 590 ug, or about 600 ug of glycine is present in the composition. In some cases, about 500 ug of glycine is present in the composition. In some cases, about 900 ug, about 910 ug, about 920 ug, about 930 ug, about 940 ug, about 950 ug, about 960 ug, about 970 ug, about 980 ug, about 990 ug, about 1000 ug, about 1010 ug, about 1020 ug, about 1030 ug, about 1040 ug, about 1050 ug, about 1060 ug, about 1070 ug, about 1080 ug, about 1090 ug, or about 1100 ug of glycine is present in the composition. In some cases, about 1000 μg of glycine is present in the composition. In some cases, about 1400 ug, about 1410 ug, about 1420 ug, about 1430 ug, about 1440 ug, about 1450 ug, about 1460 ug, about 1470 ug, about 1480 ug, about 1490 ug, about 1500 ug, about 1510 ug, about 1520 ug, about 1530 ug, about 1540 ug, about 1550 ug, about 1560 ug, about 1570 ug, about 1580 ug, about 1590 ug, or about 1600 ug of glycine is present in the composition. In some cases, about 1500 μg of glycine is present in the composition. In some cases, about 1900 ug, about 1910 ug, about 1920 ug, about 1930 ug, about 1940 ug, about 1950 ug, about 1960 ug, about 1970 ug, about 1980 ug, about 1990 ug, about 2000 ug, about 2010 ug, about 2020 ug, about 2030 ug, about 2040 ug, about 2050 ug, about 2060 ug, about 2070 ug, about 2080 ug, about 2090 ug, or about 2100 ug of glycine is present in the composition. In some cases, about 2000 μg of glycine is present in the composition. In some cases, about 2400 ug, about 2410 ug, about 2420 ug, about 2430 ug, about 2440 ug, about 2450 ug, about 2460 ug, about 2470 ug, about 2480 ug, about 2490 ug, about 2500 ug, about 2510 ug, about 2520 ug, about 2530 ug, about 2540 ug, about 2550 ug, about 2560 ug, about 2570 ug, about 2580 ug, about 2590 ug, or about 2600 ug of glycine is present in the composition. In some cases, about 2500 μg of glycine is present in the composition. In some cases, about 200 μg, about 210 μg, about 220 μg, about 230 μg, about 240 μg, about 250 μg, about 260 μg, about 270 μg, about 280 μg, about 290 μg, or about 300 μg of glycine is present in the composition.

In certain embodiments, a composition comprising a GHRH peptide comprises a delivery-enhancing agent. In certain embodiments, a composition comprising a GHRH peptide comprises a surfactant. In some cases, the delivery-enhancing agent is a surfactant. In some cases the surfactant is a delivery-enhancing agent. A surfactant and/or delivery-enhancing agent includes a nonionic surfactant, ionic surfactant, and amphoteric surfactant, and combinations thereof. In some embodiments, the surfactant comprises a nonionic surfactant. In some embodiments the surfactant and/or delivery-enhancing agent is an alkyl saccharide. In some cases, the addition of certain alkyl saccharides, when included in oral- or fast-dispersing dosage forms, modulate the proportion of compositions subject to the first-pass effect, thus allowing a fixed amount of a composition to exert greater clinical benefit, or allowing a smaller amount of an active agent to achieve similar clinical benefit compared to an otherwise larger dose. Exemplary saccharides that may be covalently joined to an alkyl, include glucose, maltose, maltotriose, maltotetrose, sucrose and trehalose. Non-limiting exemplary alkyl glycosides include octyl-, nonyl-, decyl-, undecyl-, dodecyl, tridecyl, tctradecyl, pentadecyl, octadecyl a- or β-D-maltoside,-giucoside and sucroside. In some embodiments, the glycoside comprises maltose, sucrose or glucose linked by glycosidic linkage to an alkyl chain of 9, 10, 12, 14, 16, 18 or 20 carbon atoms. In some embodiments, alkyl glycoside refers to any sugar joined by a linkage to any hydrophobic alkyl. A suitable alkyl glycoside may be one that is nontoxic and ionic/nonionic, and that it increases the absorption of a compound when it is administered with the peptide via the ocular, nasal, nasolacrimal, inhalation or pulmonary, oral cavity (sublingual or buccal cell), or CSF delivery route. Specific examples include dodecyl maltoside, tetradecyl maltoside, sucrose dodecanoate, sucrose monostearate, sucrose distearate, and/or combinations of two or more thereof. Alkyl glycosides that are particularly considered useful in embodiments of the invention include those marketed under the name Intravail® by Aegis Therapeutics, LLC, San Diego, CA.

Additional delivery-enhancing agents include glycine; aggregation inhibitory agents; charge-modifying agents; pH control agents; degradative enzyme inhibitory agents; mucolytic or mucus clearing agents; ciliostatic agents; membrane penetration-enhancing agents; modulatory agents of epithelial junction physiology; vasodilator agents; selective transport-enhancing agents; and stabilizing delivery vehicles, carriers, mucoadhesives, support or complex-forming species. Additional mucosal delivery-enhancing agents include, for example, citric acid, sodium citrate, propylene glycol, glycerin, ascorbic acid (e.g., L-ascorbic acid), sodium metabisulfite, ethylenediaminetetraacetic acid (EDTA) disodium, benzalkonium chloride, sodium hydroxide, and mixtures thereof. Delivery-enhancing agents include membrane penetration enhancing agents. Non-limiting examples of a membrane penetration enhancing agents include a surfactant; a bile salt; a phospholipid additive, mixed micelle, liposome, or carrier; an alcohol; an enamine; an NO donor compound; a long-chain amphipathic molecule; a small hydrophobic penetration enhancer; sodium or a salicylic acid derivative; a glycerol ester of acetoacetic acid; a cyclodextrin or beta-cyclodextrin derivative; a medium-chain fatty acid; a chelating agent; an amino acid or salt thereof; an N-acetylamino acid or salt thereof; an enzyme degradative to a selected membrane component; an inhibitor of fatty acid synthesis; an inhibitor of cholesterol synthesis; and any combination thereof.

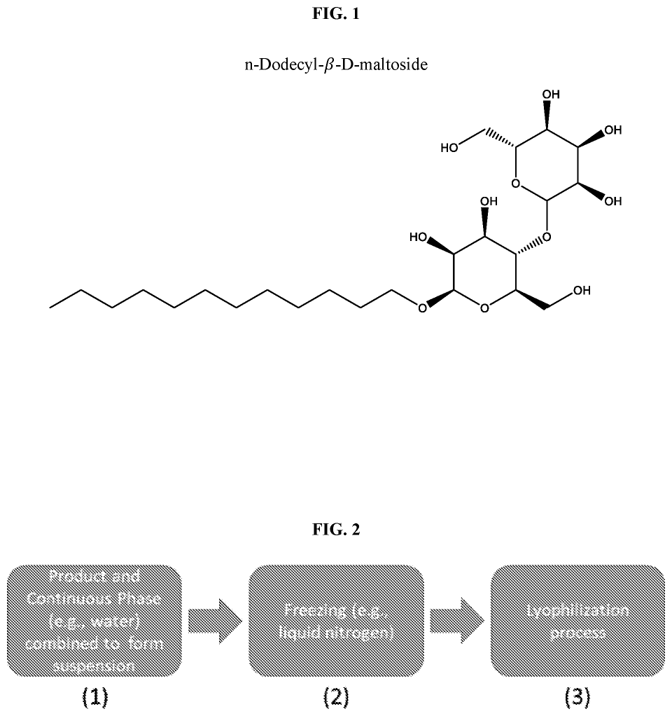

In certain embodiments, a composition comprising a GHRH peptide comprises a delivery-enhancing agent and/or surfactant comprising DDM (also provided in FIG. 1):

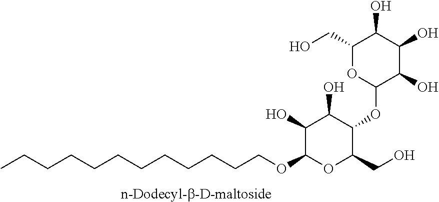 [SMILES: CCCCCCCCCCCCO[C@@H]1O[C@H](CO)[C@@H](OC2O[C@H](CO)[C@@H](O)[C@@H](O)[C@H]2O)[C@H](O)[C@@H]1O]

In some embodiments, about 1 μg to about 30 μg, about 1 μg to about 20 μg, about 1 μg to about 15 μg, about 1 μg to about 10 μg, about 5 μg to about 30 μg, about 5 μg to about 20 μg, about 5 μg to about 15 μg, or about 5 to about 10 μg of the delivery-enhancing agent is present in the composition. In some cases, about 8 μg to about 15 μg, about 8 μg to about 14 μg, about 8 μg to about 13 μg, about 8 μg to about 12 μg, about 9 μg to about 15 μg, about 9 μg to about 14 μg, about 9 μg to about 13 μg, about 9 μg to about 12 μg, about 10 μg to about 15 μg, about 10 μg to about 14 μg, about 10 μg to about 13 μg, about 10 μg to about 12 μg, or about 11 μg to about 13 μg of the delivery-enhancing agent is present in the composition. In some cases, about 8 μg, about 9 μg, about 10 μg, about 11 μg, about 12 μg, about 13 μg, about 14 μg, or about 15 μg of the delivery-enhancing agent is present in the composition.

In some embodiments, the composition comprising GHRH peptide and a delivery-enhancing agent and/or surfactant further comprises one or more agents. In some embodiments, the composition further comprises a stimulatory agent, neurotransmitter, amino acid, steroid, vitamin, or excipient, or two or more thereof.

In certain embodiments, a composition comprising a GHRH peptide comprises a stimulatory agent. In some cases, the stimulatory agent induces secretion of human growth hormone. In some cases, the stimulatory agent comprises an amino acid. In some cases, the stimulatory agent comprises a vitamin. Non-limiting examples of stimulatory agents include glycine, choline, arginine, ornithine, glycine, lysine, glutamine, or niacin (vitamin B3), and a combination of two or more thereof. In some compositions, the stimulatory agent comprises glycine. In some embodiments, about 50 μg to about 3000 μg, about 50 μg to about 2900 μg, about 50 μg to about 2800 μg, about 50 μg to about 2700 μg, about 50 μg to about 2600 μg, about 50 μg to about 2500 μg, about 50 μg to about 2400 μg, about 50 μg to about 2300 μg, about 50 μg to about 2200 μg, about 50 μg to about 2100 μg, about 50 μg to about 2000 μg, about 50 μg to about 1900 μg, about 50 μg to about 1800 μg, about 50 μg to about 1700 μg, about 50 μg to about 1600 μg, about 50 μg to about 1500 μg, about 50 μg to about 1400 μg, about 50 μg to about 1300 μg, about 50 μg to about 1200 μg, about 50 μg to about 1100 μg, about 50 μg to about 1000 μg, about 50 μg to about 900 μg, about 50 μg to about 800 μg, about 50 μg to about 700 μg, about 50 μg to about 600 μg, about 50 μg to about 500 μg, about 100 μg to about 500 μg, about 200 μg to about 500 μg, about 50 μg to about 400 μg, about 50 μg to about 300 μg, about 100 μg to about 300 μg, about 200 to about 300 μg, about 100 μg to about 3000 μg, about 200 μg to about 3000 μg, about 300 μg to about 3000 μg, about 400 μg to about 3000 μg, about 500 μg to about 3000 μg, about 600 μg to about 3000 μg, about 700 μg to about 3000 μg, about 800 μg to about 3000 μg, about 900 μg to about 3000 μg, about 1000 μg to about 3000 μg, about 1100 μg to about 3000 μg, about 1200 μg to about 3000 μg, about 1300 μg to about 3000 μg, about 1400 μg to about 3000 μg, about 1500 μg to about 3000 μg, about 1600 μg to about 3000 μg, about 1700 μg to about 3000 μg, about 1800 μg to about 3000 μg, about 1900 μg to about 3000 μg, about 2000 μg to about 3000 μg, about 2100 μg to about 3000 μg, about 2200 μg to about 3000 μg, about 2300 μg to about 3000 μg, about 2400 μg to about 3000 μg, about 2500 μg to about 3000 μg, about 2600 μg to about 3000 μg, about 2700 μg to about 3000 μg, about 2800 μg to about 3000 μg, about 2900 μg to about 3000 μg, about 100 μg to about 2500 μg, about 200 μg to about 2500 μg, about 300 μg to about 2500 μg, about 400 μg to about 2500 μg, about 500 μg to about 2500 μg, about 600 μg to about 2500 μg, about 700 μg to about 2500 μg, about 800 μg to about 2500 μg, about 900 μg to about 2500 μg, about 1000 μg to about 2500 μg, about 1100 μg to about 2500 μg, about 1200 μg to about 2500 μg, about 1300 μg to about 2500 μg, about 1400 μg to about 2500 μg, about 1500 μg to about 2500 μg, about 1600 μg to about 2500 μg, about 1700 μg to about 2500 μg, about 1800 μg to about 2500 μg, about 1900 μg to about 2500 μg, about 2000 μg to about 2500 μg, about 2100 μg to about 2500 μg, about 2200 μg to about 2500 μg, about 2300 μg to about 2500 μg, about 2400 μg to about 2500 μg, about 100 μg to about 2000 μg, about 200 μg to about 2000 μg, about 300 μg to about 2000 μg, about 400 μg to about 2000 μg, about 500 μg to about 2000 μg, about 600 μg to about 2000 μg, about 700 μg to about 2000 μg, about 800 μg to about 2000 μg, about 900 μg to about 2000 μg, about 1000 μg to about 2000 μg, about 1100 μg to about 2000 μg, about 1200 μg to about 2000 μg, about 1300 μg to about 2000 μg, about 1400 μg to about 2000 μg, about 1500 μg to about 2000 μg, about 1600 μg to about 2000 μg, about 1700 μg to about 2000 μg, about 1800 μg to about 2000 μg, about 1900 μg to about 2000 μg, about 100 μg to about 1500 μg, about 200 μg to about 1500 μg, about 300 μg to about 1500 μg, about 400 μg to about 1500 μg, about 500 μg to about 1500 μg, about 600 μg to about 1500 μg, about 700 μg to about 1500 μg, about 800 μg to about 1500 μg, about 900 μg to about 1500 μg, about 1000 μg to about 1500 μg, about 1100 μg to about 1500 μg, about 1200 μg to about 1500 μg, about 1300 μg to about 1500 μg, about 1400 μg to about 1500 μg, about 100 μg to about 1000 μg, about 200 μg to about 1000 μg, about 300 μg to about 1000 μg, about 400 μg to about 1000 μg, about 500 μg to about 1000 μg, about 600 μg to about 1000 μg, about 700 μg to about 1000 μg, about 800 μg to about 1000 μg, or about 900 μg to about 1000 μg, of stimulatory agent is present in the composition. In some embodiments, about 300 μg to about 700 μg, or about 350 μg to about 700 μg, about 400 μg to about 700 μg, about 450 μg to about 700 μg, about 500 μg to about 700 μg, about 300 μg to about 650 μg, about 350 μg to about 650 μg, about 400 μg to about 650 μg, about 450 μg to about 650 μg, about 500 μg to about 650 μg, about 300 μg to about 600 μg, about 350 μg to about 600 μg, about 400 μg to about 600 μg, about 450 μg to about 600 μg, about 500 μg to about 600 μg, about 300 μg to about 550 μg, about 350 μg to about 550 μg, about 400 μg to about 550 μg, about 450 μg to about 550 μg, about 500 μg to about 550 μg, about 300 μg to about 500 μg, about 350 μg to about 500 μg, about 400 μg to about 500 μg, or about 450 μg to about 500 μg of stimulatory agent is present in the composition. In some embodiments, about 800 μg to about 1200 μg, or about 850 μg to about 1200 μg, about 900 μg to about 1200 μg, about 950 μg to about 1200 μg, about 1000 μg to about 1200 μg, about 800 μg to about 1150 μg, about 850 μg to about 1150 μg, about 900 μg to about 1150 μg, about 950 μg to about 1150 μg, about 1000 μg to about 1150 μg, about 800 μg to about 1100 μg, about 850 μg to about 1100 μg, about 900 μg to about 1100 μg, about 950 μg to about 1100 μg, about 1000 μg to about 1100 μg, about 800 μg to about 1050 μg, about 850 μg to about 1050 μg, about 900 μg to about 1050 μg, about 950 μg to about 1050 μg, about 1000 μg to about 1050 μg, about 800 μg to about 1000 μg, about 850 μg to about 1000 μg, about 900 μg to about 1000 μg, or about 950 μg to about 1000 μg of stimulatory agent is present in the composition. In some embodiments, about 1300 μg to about 1700 μg, or about 1350 μg to about 1700 μg, about 1400 μg to about 1700 μg, about 1450 μg to about 1700 μg, about 1500 μg to about 1700 μg, about 1300 μg to about 1650 μg, about 1350 μg to about 1650 μg, about 1400 μg to about 1650 μg, about 1450 μg to about 1650 μg, about 1500 μg to about 1650 μg, about 1300 μg to about 1600 μg, about 1350 μg to about 1600 μg, about 1400 μg to about 1600 μg, about 1450 μg to about 1600 μg, about 1500 μg to about 1600 μg, about 1300 μg to about 1550 μg, about 1350 μg to about 1550 μg, about 1400 μg to about 1550 μg, about 1450 μg to about 1550 μg, about 1500 μg to about 1550 μg, about 1300 μg to about 1500 μg, about 1350 μg to about 1500 μg, about 1400 μg to about 1500 μg, or about 1450 μg to about 1500 μg of stimulatory agent is present in the composition. In some embodiments, about 1800 μg to about 2200 μg, or about 1850 μg to about 2200 μg, about 1900 μg to about 2200 μg, about 1950 μg to about 2200 μg, about 2000 μg to about 2200 μg, about 1800 μg to about 2150 μg, about 1850 μg to about 2150 μg, about 1900 μg to about 2150 μg, about 1950 μg to about 2150 μg, about 2000 μg to about 2150 μg, about 1800 μg to about 2100 μg, about 1850 μg to about 2100 μg, about 1900 μg to about 2100 μg, about 1950 μg to about 2100 μg, about 2000 μg to about 2100 μg, about 1800 μg to about 2050 μg, about 1850 μg to about 2050 μg, about 1900 μg to about 2050 μg, about 1950 μg to about 2050 μg, about 2000 μg to about 2050 μg, about 1800 μg to about 2000 μg, about 1850 μg to about 2000 μg, about 1900 μg to about 2000 μg, or about 1950 μg to about 2000 μg of stimulatory agent is present in the composition. In some embodiments, about 2300 μg to about 2700 μg, or about 2350 μg to about 2700 μg, about 2400 μg to about 2700 μg, about 2450 μg to about 2700 μg, about 2500 μg to about 2700 μg, 2300 μg to about 2650 μg, about 2350 μg to about 2650 μg, about 2400 μg to about 2650 μg, about 2450 μg to about 2650 μg, about 2500 μg to about 2650 μg, about 2300 μg to about 2600 μg, about 2350 μg to about 2600 μg, about 2400 μg to about 2600 μg, about 2450 μg to about 2600 μg, about 2500 μg to about 2600 μg, about 2300 μg to about 2550 μg, about 2350 μg to about 2550 μg, about 2400 μg to about 2550 μg, about 2450 μg to about 2550 μg, about 2500 μg to about 2550 μg, 2300 μg to about 2500 μg, about 2350 μg to about 2500 μg, about 2400 μg to about 2500 μg, or about 2450 μg to about 2500 μg of stimulatory agent is present in the composition. In some embodiments, about 400 ug, about 410 ug, about 420 ug, about 430 ug, about 440 ug, about 450 ug, about 460 ug, about 470 ug, about 480 ug, about 490 ug, about 500 ug, about 510 ug, about 520 ug, about 530 ug, about 540 ug, about 550 ug, about 560 ug, about 570 ug, about 580 ug, about 590 ug, or about 600 ug of stimulatory agent is present in the composition. In some embodiments, about 500 μg of stimulatory agent is present in the composition. In some embodiments, about 900 ug, about 910 ug, about 920 ug, about 930 ug, about 940 ug, about 950 ug, about 960 ug, about 970 ug, about 980 ug, about 990 ug, about 1000 ug, about 1010 ug, about 1020 ug, about 1030 ug, about 1040 ug, about 1050 ug, about 1060 ug, about 1070 ug, about 1080 ug, about 1090 ug, or about 1100 ug of stimulatory agent is present in the composition. In some embodiments, about 1000 μg of stimulatory agent is present in the composition. In some embodiments, about 1400 ug, about 1410 ug, about 1420 ug, about 1430 ug, about 1440 ug, about 1450 ug, about 1460 ug, about 1470 ug, about 1480 ug, about 1490 ug, about 1500 ug, about 1510 ug, about 1520 ug, about 1530 ug, about 1540 ug, about 1550 ug, about 1560 ug, about 1570 ug, about 1580 ug, about 1590 ug, or about 1600 ug of stimulatory agent is present in the composition. In some embodiments, about 1500 μg of stimulatory agent is present in the composition. In some embodiments, about 1900 ug, about 1910 ug, about 1920 ug, about 1930 ug, about 1940 ug, about 1950 ug, about 1960 ug, about 1970 ug, about 1980 ug, about 1990 ug, about 2000 ug, about 2010 ug, about 2020 ug, about 2030 ug, about 2040 ug, about 2050 ug, about 2060 ug, about 2070 ug, about 2080 ug, about 2090 ug, or about 2100 ug of stimulatory agent is present in the composition. In some embodiments, about 2000 μg of stimulatory agent is present in the composition. In some embodiments, about 2400 ug, about 2410 ug, about 2420 ug, about 2430 ug, about 2440 ug, about 2450 ug, about 2460 ug, about 2470 ug, about 2480 ug, about 2490 ug, about 2500 ug, about 2510 ug, about 2520 ug, about 2530 ug, about 2540 ug, about 2550 ug, about 2560 ug, about 2570 ug, about 2580 ug, about 2590 ug, or about 2600 ug of stimulatory agent is present in the composition. In some embodiments, about 2500 μg of stimulatory agent is present in the composition. In some embodiments, about 50 μg to about 500 μg, about 100 μg to about 500 μg, about 200 μg to about 500 μg, about 50 μg to about 400 μg, about 50 μg to about 300 μg, about 100 μg to about 300 μg, or about 200 to about 300 μg of the stimulatory agent is present in the composition. In some cases, about 200 μg to about 300 μg, or about 225 μg to about 300 μg, about 250 μg to about 300 μg, about 200 to about 275 μg, about 200 to about 250 μg, or about 225 μg to about 275 μg of the stimulatory agent is present in the composition. In some cases, about 200 μg, about 210 μg, about 220 μg, about 230 μg, about 240 μg, about 250 μg, about 260 μg, about 270 μg, about 280 μg, about 290 μg, or about 300 μg of the stimulatory agent is present in the composition. In some embodiments, the composition further comprises a delivery-enhancing agent, surfactant, alkyl glycoside, neurotransmitter, amino acid, steroid, vitamin, or excipient, or two or more thereof.

In certain embodiments, a composition comprising a GHRH peptide comprises an amino acid. In some cases, the amino acid induces secretion of human growth hormone. In some cases, the amino acid is a stimulatory agent. Non-limiting examples of amino acids include glycine, arginine, ornithine, glycine, lysine, glutamine, and a combination of two or more thereof. In some compositions, the amino acid comprises glycine. In some embodiments, about 50 μg to about 3000 μg, about 50 μg to about 2900 μg, about 50 μg to about 2800 μg, about 50 μg to about 2700 μg, about 50 μg to about 2600 μg, about 50 μg to about 2500 μg, about 50 μg to about 2400 μg, about 50 μg to about 2300 μg, about 50 μg to about 2200 μg, about 50 μg to about 2100 μg, about 50 μg to about 2000 μg, about 50 μg to about 1900 μg, about 50 μg to about 1800 μg, about 50 μg to about 1700 μg, about 50 μg to about 1600 μg, about 50 μg to about 1500 μg, about 50 μg to about 1400 μg, about 50 μg to about 1300 μg, about 50 μg to about 1200 μg, about 50 μg to about 1100 μg, about 50 μg to about 1000 μg, about 50 μg to about 900 μg, about 50 μg to about 800 μg, about 50 μg to about 700 μg, about 50 μg to about 600 μg, about 50 μg to about 500 μg, about 100 μg to about 500 μg, about 200 μg to about 500 μg, about 50 μg to about 400 μg, about 50 μg to about 300 μg, about 100 μg to about 300 μg, about 200 to about 300 μg, about 100 μg to about 3000 μg, about 200 μg to about 3000 μg, about 300 μg to about 3000 μg, about 400 μg to about 3000 μg, about 500 μg to about 3000 μg, about 600 μg to about 3000 μg, about 700 μg to about 3000 μg, about 800 μg to about 3000 μg, about 900 μg to about 3000 μg, about 1000 μg to about 3000 μg, about 1100 μg to about 3000 μg, about 1200 μg to about 3000 μg, about 1300 μg to about 3000 μg, about 1400 μg to about 3000 μg, about 1500 μg to about 3000 μg, about 1600 μg to about 3000 μg, about 1700 μg to about 3000 μg, about 1800 μg to about 3000 μg, about 1900 μg to about 3000 μg, about 2000 μg to about 3000 μg, about 2100 μg to about 3000 μg, about 2200 μg to about 3000 μg, about 2300 μg to about 3000 μg, about 2400 μg to about 3000 μg, about 2500 μg to about 3000 μg, about 2600 μg to about 3000 μg, about 2700 μg to about 3000 μg, about 2800 μg to about 3000 μg, about 2900 μg to about 3000 μg, about 100 μg to about 2500 μg, about 200 μg to about 2500 μg, about 300 μg to about 2500 μg, about 400 μg to about 2500 μg, about 500 μg to about 2500 μg, about 600 μg to about 2500 μg, about 700 μg to about 2500 μg, about 800 μg to about 2500 μg, about 900 μg to about 2500 μg, about 1000 μg to about 2500 μg, about 1100 μg to about 2500 μg, about 1200 μg to about 2500 μg, about 1300 μg to about 2500 μg, about 1400 μg to about 2500 μg, about 1500 μg to about 2500 μg, about 1600 μg to about 2500 μg, about 1700 μg to about 2500 μg, about 1800 μg to about 2500 μg, about 1900 μg to about 2500 μg, about 2000 μg to about 2500 μg, about 2100 μg to about 2500 μg, about 2200 μg to about 2500 μg, about 2300 μg to about 2500 μg, about 2400 μg to about 2500 μg, about 100 μg to about 2000 μg, about 200 μg to about 2000 μg, about 300 μg to about 2000 μg, about 400 μg to about 2000 μg, about 500 μg to about 2000 μg, about 600 μg to about 2000 μg, about 700 μg to about 2000 μg, about 800 μg to about 2000 μg, about 900 μg to about 2000 μg, about 1000 μg to about 2000 μg, about 1100 μg to about 2000 μg, about 1200 μg to about 2000 μg, about 1300 μg to about 2000 μg, about 1400 μg to about 2000 μg, about 1500 μg to about 2000 μg, about 1600 μg to about 2000 μg, about 1700 μg to about 2000 μg, about 1800 μg to about 2000 μg, about 1900 μg to about 2000 μg, about 100 μg to about 1500 μg, about 200 μg to about 1500 μg, about 300 μg to about 1500 μg, about 400 μg to about 1500 μg, about 500 μg to about 1500 μg, about 600 μg to about 1500 μg, about 700 μg to about 1500 μg, about 800 μg to about 1500 μg, about 900 μg to about 1500 μg, about 1000 μg to about 1500 μg, about 1100 μg to about 1500 μg, about 1200 μg to about 1500 μg, about 1300 μg to about 1500 μg, about 1400 μg to about 1500 μg, about 100 μg to about 1000 μg, about 200 μg to about 1000 μg, about 300 μg to about 1000 μg, about 400 μg to about 1000 μg, about 500 μg to about 1000 μg, about 600 μg to about 1000 μg, about 700 μg to about 1000 μg, about 800 μg to about 1000 μg, or about 900 μg to about 1000 μg, of amino acid is present in the composition. In some embodiments, about 300 μg to about 700 μg, or about 350 μg to about 700 μg, about 400 μg to about 700 μg, about 450 μg to about 700 μg, about 500 μg to about 700 μg, about 300 μg to about 650 μg, about 350 μg to about 650 μg, about 400 μg to about 650 μg, about 450 μg to about 650 μg, about 500 μg to about 650 μg, about 300 μg to about 600 μg, about 350 μg to about 600 μg, about 400 μg to about 600 μg, about 450 μg to about 600 μg, about 500 μg to about 600 μg, about 300 μg to about 550 μg, about 350 μg to about 550 μg, about 400 μg to about 550 μg, about 450 μg to about 550 μg, about 500 μg to about 550 μg, about 300 μg to about 500 μg, about 350 μg to about 500 μg, about 400 μg to about 500 μg, or about 450 μg to about 500 μg of amino acid is present in the composition. In some embodiments, about 800 μg to about 1200 μg, or about 850 μg to about 1200 μg, about 900 μg to about 1200 μg, about 950 μg to about 1200 μg, about 1000 μg to about 1200 μg, about 800 μg to about 1150 μg, about 850 μg to about 1150 μg, about 900 μg to about 1150 μg, about 950 μg to about 1150 μg, about 1000 μg to about 1150 μg, about 800 μg to about 1100 μg, about 850 μg to about 1100 μg, about 900 μg to about 1100 μg, about 950 μg to about 1100 μg, about 1000 μg to about 1100 μg, about 800 μg to about 1050 μg, about 850 μg to about 1050 μg, about 900 μg to about 1050 μg, about 950 μg to about 1050 μg, about 1000 μg to about 1050 μg, about 800 μg to about 1000 μg, about 850 μg to about 1000 μg, about 900 μg to about 1000 μg, or about 950 μg to about 1000 μg of amino acid is present in the composition. In some embodiments, about 1300 μg to about 1700 μg, or about 1350 μg to about 1700 μg, about 1400 μg to about 1700 μg, about 1450 μg to about 1700 μg, about 1500 μg to about 1700 μg, about 1300 μg to about 1650 μg, about 1350 μg to about 1650 μg, about 1400 μg to about 1650 μg, about 1450 μg to about 1650 μg, about 1500 μg to about 1650 μg, about 1300 μg to about 1600 μg, about 1350 μg to about 1600 μg, about 1400 μg to about 1600 μg, about 1450 μg to about 1600 μg, about 1500 μg to about 1600 μg, about 1300 μg to about 1550 μg, about 1350 μg to about 1550 μg, about 1400 μg to about 1550 μg, about 1450 μg to about 1550 μg, about 1500 μg to about 1550 μg, about 1300 μg to about 1500 μg, about 1350 μg to about 1500 μg, about 1400 μg to about 1500 μg, or about 1450 μg to about 1500 μg of amino acid is present in the composition. In some embodiments, about 1800 μg to about 2200 μg, or about 1850 μg to about 2200 μg, about 1900 μg to about 2200 μg, about 1950 μg to about 2200 μg, about 2000 μg to about 2200 μg, about 1800 μg to about 2150 μg, about 1850 μg to about 2150 μg, about 1900 μg to about 2150 μg, about 1950 μg to about 2150 μg, about 2000 μg to about 2150 μg, about 1800 μg to about 2100 μg, about 1850 μg to about 2100 μg, about 1900 μg to about 2100 μg, about 1950 μg to about 2100 μg, about 2000 μg to about 2100 μg, about 1800 μg to about 2050 μg, about 1850 μg to about 2050 μg, about 1900 μg to about 2050 μg, about 1950 μg to about 2050 μg, about 2000 μg to about 2050 μg, about 1800 μg to about 2000 μg, about 1850 μg to about 2000 μg, about 1900 μg to about 2000 μg, or about 1950 μg to about 2000 μg of amino acid is present in the composition. In some embodiments, about 2300 μg to about 2700 μg, or about 2350 μg to about 2700 μg, about 2400 μg to about 2700 μg, about 2450 μg to about 2700 μg, about 2500 μg to about 2700 μg, 2300 μg to about 2650 μg, about 2350 μg to about 2650 μg, about 2400 μg to about 2650 μg, about 2450 μg to about 2650 μg, about 2500 μg to about 2650 μg, about 2300 μg to about 2600 μg, about 2350 μg to about 2600 μg, about 2400 μg to about 2600 μg, about 2450 μg to about 2600 μg, about 2500 μg to about 2600 μg, about 2300 μg to about 2550 μg, about 2350 μg to about 2550 μg, about 2400 μg to about 2550 μg, about 2450 μg to about 2550 μg, about 2500 μg to about 2550 μg, 2300 μg to about 2500 μg, about 2350 μg to about 2500 μg, about 2400 μg to about 2500 μg, or about 2450 μg to about 2500 μg of amino acid is present in the composition. In some embodiments, about 400 ug, about 410 ug, about 420 ug, about 430 ug, about 440 ug, about 450 ug, about 460 ug, about 470 ug, about 480 ug, about 490 ug, about 500 ug, about 510 ug, about 520 ug, about 530 ug, about 540 ug, about 550 ug, about 560 ug, about 570 ug, about 580 ug, about 590 ug, or about 600 ug of amino acid is present in the composition. In some embodiments, about 500 μg of amino acid is present in the composition. In some embodiments, about 900 ug, about 910 ug, about 920 ug, about 930 ug, about 940 ug, about 950 ug, about 960 ug, about 970 ug, about 980 ug, about 990 ug, about 1000 ug, about 1010 ug, about 1020 ug, about 1030 ug, about 1040 ug, about 1050 ug, about 1060 ug, about 1070 ug, about 1080 ug, about 1090 ug, or about 1100 ug of amino acid is present in the composition. In some embodiments, about 1000 μg of amino acid is present in the composition. In some embodiments, about 1400 ug, about 1410 ug, about 1420 ug, about 1430 ug, about 1440 ug, about 1450 ug, about 1460 ug, about 1470 ug, about 1480 ug, about 1490 ug, about 1500 ug, about 1510 ug, about 1520 ug, about 1530 ug, about 1540 ug, about 1550 ug, about 1560 ug, about 1570 ug, about 1580 ug, about 1590 ug, or about 1600 ug of amino acid is present in the composition. In some embodiments, about 1500 μg of amino acid is present in the composition. In some embodiments, about 1900 ug, about 1910 ug, about 1920 ug, about 1930 ug, about 1940 ug, about 1950 ug, about 1960 ug, about 1970 ug, about 1980 ug, about 1990 ug, about 2000 ug, about 2010 ug, about 2020 ug, about 2030 ug, about 2040 ug, about 2050 ug, about 2060 ug, about 2070 ug, about 2080 ug, about 2090 ug, or about 2100 ug of amino acid is present in the composition. In some embodiments, about 2000 μg of amino acid is present in the composition. In some embodiments, about 2400 ug, about 2410 ug, about 2420 ug, about 2430 ug, about 2440 ug, about 2450 ug, about 2460 ug, about 2470 ug, about 2480 ug, about 2490 ug, about 2500 ug, about 2510 ug, about 2520 ug, about 2530 ug, about 2540 ug, about 2550 ug, about 2560 ug, about 2570 ug, about 2580 ug, about 2590 ug, or about 2600 ug of amino acid is present in the composition. In some embodiments, about 2500 μg of amino acid is present in the composition. In some embodiments, about 50 μg to about 500 μg, about 100 μg to about 500 μg, about 200 μg to about 500 μg, about 50 μg to about 400 μg, about 50 μg to about 300 μg, about 100 μg to about 300 μg, or about 200 to about 300 μg of the amino acid is present in the composition. In some cases, about 200 μg to about 300 μg, or about 225 μg to about 300 μg, about 250 μg to about 300 μg, about 200 to about 275 μg, about 200 to about 250 μg, or about 225 μg to about 275 μg of the amino acid is present in the composition. In some cases, about 200 μg, about 210 μg, about 220 μg, about 230 μg, about 240 μg, about 250 μg, about 260 μg, about 270 μg, about 280 μg, about 290 μg, or about 300 μg of the amino acid is present in the composition. In some embodiments, the composition further comprises a delivery-enhancing agent, surfactant, alkyl glycoside, stimulatory agent, neurotransmitter, steroid, vitamin, or excipient, or two or more thereof.

In certain embodiments, a composition comprising a GHRH peptide comprises a neurotransmitter. Non-limiting examples of neurotransmitters include acetylcholine, dopamine, glutamate, serotonin, norepinephrine, gamma-aminobutyric acid (GABA), glycine, corticotropin-releasing factor (CRF), galanin, encephalin, enkephalin, dynorphin, CRH, dynorphin, and neuropeptide Y. Certain amino acids act as neurotransmitters including GABA, aspartate, N-methyl-D-aspartate, and glycine. In some embodiments an amino acid neurotransmitter acts as a stimulatory agent. Growth hormone secretion is mainly regulated by hypophysiotropic neurohormones, including GHRH. Many neurotransmitters, such as acetylcholine, arginine, galanin, and glycine, play a stimulatory role in growth hormone secretion. In certain embodiments, the role of the neurotransmitter and/or stimulatory agent is to induce the secretion of human growth hormone. In some cases, the neurotransmitter is an amino acid. In some cases, the neurotransmitter is a stimulatory agent. In some cases, the amino acid is a neurotransmitter. In some cases, the stimulatory agent is a neurotransmitter. In certain embodiments the composition comprising a GHRH peptide comprises an amino acid and/or neurotransmitter and/or stimulatory agent such as choline, arginine, ornithine, glycine, lysine, glutamine, or niacin, or a combination of two or more thereof. In some cases, the amino acid and/or neurotransmitter and/or stimulatory agent induces secretion of human growth hormone. Non-limiting examples of amino acids include glycine, arginine, ornithine, glycine, lysine, glutamine, and a combination of two or more thereof. In some compositions, the amino acid comprises glycine. In some embodiments, about 50 μg to about 3000 μg, about 50 μg to about 2900 μg, about 50 μg to about 2800 μg, about 50 μg to about 2700 μg, about 50 μg to about 2600 μg, about 50 μg to about 2500 μg, about 50 μg to about 2400 μg, about 50 μg to about 2300 μg, about 50 μg to about 2200 μg, about 50 μg to about 2100 μg, about 50 μg to about 2000 μg, about 50 μg to about 1900 μg, about 50 μg to about 1800 μg, about 50 μg to about 1700 μg, about 50 μg to about 1600 μg, about 50 μg to about 1500 μg, about 50 μg to about 1400 μg, about 50 μg to about 1300 μg, about 50 μg to about 1200 μg, about 50 μg to about 1100 μg, about 50 μg to about 1000 μg, about 50 μg to about 900 μg, about 50 μg to about 800 μg, about 50 μg to about 700 μg, about 50 μg to about 600 μg, about 50 μg to about 500 μg, about 100 μg to about 500 μg, about 200 μg to about 500 μg, about 50 μg to about 400 μg, about 50 μg to about 300 μg, about 100 μg to about 300 μg, about 200 to about 300 μg, about 100 μg to about 3000 μg, about 200 μg to about 3000 μg, about 300 μg to about 3000 μg, about 400 μg to about 3000 μg, about 500 μg to about 3000 μg, about 600 μg to about 3000 μg, about 700 μg to about 3000 μg, about 800 μg to about 3000 μg, about 900 μg to about 3000 μg, about 1000 μg to about 3000 μg, about 1100 μg to about 3000 μg, about 1200 μg to about 3000 μg, about 1300 μg to about 3000 μg, about 1400 μg to about 3000 μg, about 1500 μg to about 3000 μg, about 1600 μg to about 3000 μg, about 1700 μg to about 3000 μg, about 1800 μg to about 3000 μg, about 1900 μg to about 3000 μg, about 2000 μg to about 3000 μg, about 2100 μg to about 3000 μg, about 2200 μg to about 3000 μg, about 2300 μg to about 3000 μg, about 2400 μg to about 3000 μg, about 2500 μg to about 3000 μg, about 2600 μg to about 3000 μg, about 2700 μg to about 3000 μg, about 2800 μg to about 3000 μg, about 2900 μg to about 3000 μg, about 100 μg to about 2500 μg, about 200 μg to about 2500 μg, about 300 μg to about 2500 μg, about 400 μg to about 2500 μg, about 500 μg to about 2500 μg, about 600 μg to about 2500 μg, about 700 μg to about 2500 μg, about 800 μg to about 2500 μg, about 900 μg to about 2500 μg, about 1000 μg to about 2500 μg, about 1100 μg to about 2500 μg, about 1200 μg to about 2500 μg, about 1300 μg to about 2500 μg, about 1400 μg to about 2500 μg, about 1500 μg to about 2500 μg, about 1600 μg to about 2500 μg, about 1700 μg to about 2500 μg, about 1800 μg to about 2500 μg, about 1900 μg to about 2500 μg, about 2000 μg to about 2500 μg, about 2100 μg to about 2500 μg, about 2200 μg to about 2500 μg, about 2300 μg to about 2500 μg, about 2400 μg to about 2500 μg, about 100 μg to about 2000 μg, about 200 μg to about 2000 μg, about 300 μg to about 2000 μg, about 400 μg to about 2000 μg, about 500 μg to about 2000 μg, about 600 μg to about 2000 μg, about 700 μg to about 2000 μg, about 800 μg to about 2000 μg, about 900 μg to about 2000 μg, about 1000 μg to about 2000 μg, about 1100 μg to about 2000 μg, about 1200 μg to about 2000 μg, about 1300 μg to about 2000 μg, about 1400 μg to about 2000 μg, about 1500 μg to about 2000 μg, about 1600 μg to about 2000 μg, about 1700 μg to about 2000 μg, about 1800 μg to about 2000 μg, about 1900 μg to about 2000 μg, about 100 μg to about 1500 μg, about 200 μg to about 1500 μg, about 300 μg to about 1500 μg, about 400 μg to about 1500 μg, about 500 μg to about 1500 μg, about 600 μg to about 1500 μg, about 700 μg to about 1500 μg, about 800 μg to about 1500 μg, about 900 μg to about 1500 μg, about 1000 μg to about 1500 μg, about 1100 μg to about 1500 μg, about 1200 μg to about 1500 μg, about 1300 μg to about 1500 μg, about 1400 μg to about 1500 μg, about 100 μg to about 1000 μg, about 200 μg to about 1000 μg, about 300 μg to about 1000 μg, about 400 μg to about 1000 μg, about 500 μg to about 1000 μg, about 600 μg to about 1000 μg, about 700 μg to about 1000 μg, about 800 μg to about 1000 μg, or about 900 μg to about 1000 μg, of amino acid and/or neurotransmitter and/or stimulatory agent is present in the composition. In some embodiments, about 300 μg to about 700 μg, or about 350 μg to about 700 μg, about 400 μg to about 700 μg, about 450 μg to about 700 μg, about 500 μg to about 700 μg, about 300 μg to about 650 μg, about 350 μg to about 650 μg, about 400 μg to about 650 μg, about 450 μg to about 650 μg, about 500 μg to about 650 μg, about 300 μg to about 600 μg, about 350 μg to about 600 μg, about 400 μg to about 600 μg, about 450 μg to about 600 μg, about 500 μg to about 600 μg, about 300 μg to about 550 μg, about 350 μg to about 550 μg, about 400 μg to about 550 μg, about 450 μg to about 550 μg, about 500 μg to about 550 μg, about 300 μg to about 500 μg, about 350 μg to about 500 μg, about 400 μg to about 500 μg, or about 450 μg to about 500 μg of amino acid and/or neurotransmitter and/or stimulatory agent is present in the composition. In some embodiments, about 800 μg to about 1200 μg, or about 850 μg to about 1200 μg, about 900 μg to about 1200 μg, about 950 μg to about 1200 μg, about 1000 μg to about 1200 μg, about 800 μg to about 1150 μg, about 850 μg to about 1150 μg, about 900 μg to about 1150 μg, about 950 μg to about 1150 μg, about 1000 μg to about 1150 μg, about 800 μg to about 1100 μg, about 850 μg to about 1100 μg, about 900 μg to about 1100 μg, about 950 μg to about 1100 μg, about 1000 μg to about 1100 μg, about 800 μg to about 1050 μg, about 850 μg to about 1050 μg, about 900 μg to about 1050 μg, about 950 μg to about 1050 μg, about 1000 μg to about 1050 μg, about 800 μg to about 1000 μg, about 850 μg to about 1000 μg, about 900 μg to about 1000 μg, or about 950 μg to about 1000 μg of amino acid and/or neurotransmitter and/or stimulatory agent is present in the composition. In some embodiments, about 1300 μg to about 1700 μg, or about 1350 μg to about 1700 μg, about 1400 μg to about 1700 μg, about 1450 μg to about 1700 μg, about 1500 μg to about 1700 μg, about 1300 μg to about 1650 μg, about 1350 μg to about 1650 μg, about 1400 μg to about 1650 μg, about 1450 μg to about 1650 μg, about 1500 μg to about 1650 μg, about 1300 μg to about 1600 μg, about 1350 μg to about 1600 μg, about 1400 μg to about 1600 μg, about 1450 μg to about 1600 μg, about 1500 μg to about 1600 μg, about 1300 μg to about 1550 μg, about 1350 μg to about 1550 μg, about 1400 μg to about 1550 μg, about 1450 μg to about 1550 μg, about 1500 μg to about 1550 μg, about 1300 μg to about 1500 μg, about 1350 μg to about 1500 μg, about 1400 μg to about 1500 μg, or about 1450 μg to about 1500 μg of amino acid and/or neurotransmitter and/or stimulatory agent is present in the composition. In some embodiments, about 1800 μg to about 2200 μg, or about 1850 μg to about 2200 μg, about 1900 μg to about 2200 μg, about 1950 μg to about 2200 μg, about 2000 μg to about 2200 μg, about 1800 μg to about 2150 μg, about 1850 μg to about 2150 μg, about 1900 μg to about 2150 μg, about 1950 μg to about 2150 μg, about 2000 μg to about 2150 μg, about 1800 μg to about 2100 μg, about 1850 μg to about 2100 μg, about 1900 μg to about 2100 μg, about 1950 μg to about 2100 μg, about 2000 μg to about 2100 μg, about 1800 μg to about 2050 μg, about 1850 μg to about 2050 μg, about 1900 μg to about 2050 μg, about 1950 μg to about 2050 μg, about 2000 μg to about 2050 μg, about 1800 μg to about 2000 μg, about 1850 μg to about 2000 μg, about 1900 μg to about 2000 μg, or about 1950 μg to about 2000 μg of amino acid and/or neurotransmitter and/or stimulatory agent is present in the composition. In some embodiments, about 2300 μg to about 2700 μg, or about 2350 μg to about 2700 μg, about 2400 μg to about 2700 μg, about 2450 μg to about 2700 μg, about 2500 μg to about 2700 μg, 2300 μg to about 2650 μg, about 2350 μg to about 2650 μg, about 2400 μg to about 2650 μg, about 2450 μg to about 2650 μg, about 2500 μg to about 2650 μg, about 2300 μg to about 2600 μg, about 2350 μg to about 2600 μg, about 2400 μg to about 2600 μg, about 2450 μg to about 2600 μg, about 2500 μg to about 2600 μg, about 2300 μg to about 2550 μg, about 2350 μg to about 2550 μg, about 2400 μg to about 2550 μg, about 2450 μg to about 2550 μg, about 2500 μg to about 2550 μg, 2300 μg to about 2500 μg, about 2350 μg to about 2500 μg, about 2400 μg to about 2500 μg, or about 2450 μg to about 2500 μg of amino acid and/or neurotransmitter and/or stimulatory agent is present in the composition. In some embodiments, about 400 ug, about 410 ug, about 420 ug, about 430 ug, about 440 ug, about 450 ug, about 460 ug, about 470 ug, about 480 ug, about 490 ug, about 500 ug, about 510 ug, about 520 ug, about 530 ug, about 540 ug, about 550 ug, about 560 ug, about 570 ug, about 580 ug, about 590 ug, or about 600 ug of amino acid and/or neurotransmitter and/or stimulatory agent is present in the composition. In some embodiments, about 500 μg of amino acid and/or neurotransmitter and/or stimulatory agent is present in the composition. In some embodiments, about 900 ug, about 910 ug, about 920 ug, about 930 ug, about 940 ug, about 950 ug, about 960 ug, about 970 ug, about 980 ug, about 990 ug, about 1000 ug, about 1010 ug, about 1020 ug, about 1030 ug, about 1040 ug, about 1050 ug, about 1060 ug, about 1070 ug, about 1080 ug, about 1090 ug, or about 1100 ug of amino acid and/or neurotransmitter and/or stimulatory agent is present in the composition. In some embodiments, about 1000 μg of amino acid and/or neurotransmitter and/or stimulatory agent is present in the composition. In some embodiments, about 1400 ug, about 1410 ug, about 1420 ug, about 1430 ug, about 1440 ug, about 1450 ug, about 1460 ug, about 1470 ug, about 1480 ug, about 1490 ug, about 1500 ug, about 1510 ug, about 1520 ug, about 1530 ug, about 1540 ug, about 1550 ug, about 1560 ug, about 1570 ug, about 1580 ug, about 1590 ug, or about 1600 ug of amino acid and/or neurotransmitter and/or stimulatory agent is present in the composition. In some embodiments, about 1500 μg of amino acid and/or neurotransmitter and/or stimulatory agent is present in the composition. In some embodiments, about 1900 ug, about 1910 ug, about 1920 ug, about 1930 ug, about 1940 ug, about 1950 ug, about 1960 ug, about 1970 ug, about 1980 ug, about 1990 ug, about 2000 ug, about 2010 ug, about 2020 ug, about 2030 ug, about 2040 ug, about 2050 ug, about 2060 ug, about 2070 ug, about 2080 ug, about 2090 ug, or about 2100 ug of amino acid and/or neurotransmitter and/or stimulatory agent is present in the composition. In some embodiments, about 1000 μg of amino acid and/or neurotransmitter and/or stimulatory agent is present in the composition. In some embodiments, about 2400 ug, about 2410 ug, about 2420 ug, about 2430 ug, about 2440 ug, about 2450 ug, about 2460 ug, about 2470 ug, about 2480 ug, about 2490 ug, about 2500 ug, about 2510 ug, about 2520 ug, about 2530 ug, about 2540 ug, about 2550 ug, about 2560 ug, about 2570 ug, about 2580 ug, about 2590 ug, or about 2600 ug of amino acid and/or neurotransmitter and/or stimulatory agent is present in the composition. In some embodiments, about 2500 μg of amino acid and/or neurotransmitter and/or stimulatory agent is present in the composition. In some embodiments, about 50 μg to about 500 μg, about 100 μg to about 500 μg, about 200 μg to about 500 μg, about 50 μg to about 400 μg, about 50 μg to about 300 μg, about 100 μg to about 300 μg, or about 200 to about 300 μg of the neurotransmitter is present in the composition. In some cases, about 200 μg to about 300 μg, or about 225 μg to about 300 μg, about 250 μg to about 300 μg, about 200 to about 275 μg, about 200 to about 250 μg, or about 225 μg to about 275 μg of the neurotransmitter is present in the composition. In some cases, about 200 μg, about 210 μg, about 220 μg, about 230 μg, about 240 μg, about 250 μg, about 260 μg, about 270 μg, about 280 μg, about 290 μg, or about 300 μg of the neurotransmitter is present in the composition. In some embodiments, the composition further comprises a delivery-enhancing agent, surfactant, alkyl glycoside, stimulatory agent, amino acid, steroid, vitamin, or excipient, or two or more thereof.

In certain embodiments a composition comprising a GHRH peptide comprises a steroid, including certain steroidal glycosides. Steroidal glycosides are naturally occurring sugar conjugates of C27 steroidal compounds. The aglycone of a steroid saponin is usually a spirostanol or a furostanol. The glycone parts of these compounds are mostly oligosaccharides, arranged either in a linear or branched fashion, attached to hydroxyl groups through an acetal linkage (2, 3). The basic steroid saponins, another class of saponins, contain nitrogen analogs of steroid sapogenins as aglycones. Steroidal glycosides include groups of biologically active steroid saponin groups found in ginseng that have been shown to exert pharmacological effects. Saponins are a group of naturally occurring plant glycosides, characterized by their strong foam-forming properties in aqueous solution. Ginseng is consumed as a health supplement due to the presence of pharmacologically active ginsenoside saponins. About 150 ginsenoside saponins are known, and more than 90% of ginsenoside saponins are classified as Rb1, Rb2, Rc, Rd, Re, Rg1, and Rg3. Rg3 ginsenoside is known to have antioxidant, antiaging, anti-inflammatory, and anticancer properties. In certain embodiments, the steroid comprises a ginsenoside. In some embodiments the ginsenoside comprises ginsenoside Rb1, ginsenoside Rb2, ginsenoside Rb3, ginsenoside Rc, ginsenoside Rd, ginsenoside Re, ginsenoside Rg1, ginsenoside Rg2, ginsenoside Rg3, or ginsenoside Rh1, or a combination of two or more thereof. In certain embodiments the ginsenoside comprises Rg3 ginsenosides. In some embodiments, about 200 μg to about 2000 μg, about 500 μg to about 2000 μg, about 500 μg to about 1500 μg, about 500 μg to about 1000 μg, about 1000 μg to about 2000 μg, or about 1000 to about 1500 μg of the steroid is present in the composition. In some cases, about 800 μg to about 1200 μg, or about 900 μg to about 1100 μg, or about 950 μg to about 1050 μg of the steroid is present in the composition. In some cases, about 900 μg, about 950 μg, about 1000 μg, about 1050 μg, or about 1100 μg of the steroid is present in the composition. In some embodiments, the composition further comprises a delivery-enhancing agent, surfactant, alkyl glycoside, stimulatory agent, neurotransmitter, amino acid, vitamin, or excipient, or two or more thereof.

In certain embodiments, a composition comprising a GHRH peptide comprises a vitamin. Non-limiting examples of vitamins include vitamins: A, C, D, E, K, B1, B2, B3, B5, B6, B7, B9, and B12. Additional vitamins include pyridoxine, cyanocobalamin, ascorbic acid, biotin niacin, folate, pantothenic acid, riboflavin, thiamine, choline, carnitine, and folic acid. Many vitamins are required for the body to work properly, and serve important functions and work as, as non-limiting examples, antioxidants, and coagulants. Non-limiting sources for vitamins include methylcobalamin, adenosylcobalamin, hydroxycobalamin, nicotinamide riboside, nicotinamide adenine dinucleotide, nicotinamide, and nicotinic acid. In some cases, the vitamin is methylcobalamin, which has a high bioavailability. In some embodiments, a vitamin is a vitamin analog. In some embodiments the vitamins or vitamin analogs serve functions such as improving pathophysiological conditions, metabolic pathways, improving bioavailability of the composition, increased absorption of the composition, and increased NAD+ levels. In some embodiments the vitamin comprises vitamin B3 or forms of vitamin B3. Nicotinamide riboside is a pyridine-nucleoside and a form vitamin B3. It is a precursor to NAD+. Nicotinamide riboside has numerous potential health benefits mediated via elevated levels of NAD+ content in the body. NAD+ is an essential coenzyme responsible for important roles in various metabolic pathways. Increasing the overall content of NAD+ has been confirmed to be a valuable strategy for treating manypathophysiological conditions. The health benefits of nicotinamide riboside have facilitated numerous animal and human studies for the treatment of several metabolic, cardiovascular, and neurodegenerative disorders. Moreover, nicotinamide riboside has oral availability. In some embodiments the vitamin comprises vitamin B12 or forms of vitamin B12. Natural forms of vitamin B12 are commercially available, such as methylcobalamin, adenosylcobalamin, and hydroxycobalamin. All have been shown in clinical studies to improve vitamin B12 status. These commercially available B12 vitamins are bioidentical to the B12 forms occurring in human physiology and in animal foods. There are also synthetic forms such as cyanocobalamin, a synthetic B12 compound used for food fortification and in some supplements. Cyanocobalamin occurs only in trace amounts in human tissues, usually resulting from cyanide intake from smoking or other sources. All of the following forms have been shown to improve vitamin B12 status. In certain embodiments a composition comprising a GHRH peptide comprises vitamin B3. In non-limiting embodiments, the vitamin B3 component comprises nicotinamide or niacin. In further non-limiting embodiments the nicotinamide comprises nicotinamide riboside. In certain embodiments a composition comprising a GHRH peptide comprises vitamin B12. In non-limiting embodiments, the vitamin B12 component comprises methylcobalamin, cyanocobalamin, adenosylcobalamin, or hydroxycobalamin. In further non-limiting embodiments, the vitamin B12 component comprises methylcobalamin. In a non-limiting embodiment, the composition comprises two vitamins, e.g., nicotinamide riboside and methylcobalamin. In some embodiments, the composition further comprises a delivery-enhancing agent, surfactant, alkyl glycoside, stimulatory agent, neurotransmitter, amino acid, steroid, or excipient, or two or more thereof.

In some embodiments, about 200 μg to about 2000 μg, about 500 μg to about 2000 μg, about 500 μg to about 1500 μg, about 500 μg to about 1000 μg, about 1000 μg to about 2000 μg, or about 1000 to about 1500 μg of the vitamin is present in the composition. In some cases, about 800 μg to about 1200 μg, or about 900 μg to about 1100 μg, or about 950 μg to about 1050 μg of the vitamin is present in the composition. In some cases, about 900 μg, about 950 μg, about 1000 μg, about 1050 μg, or about 1100 μg of the vitamin is present in the composition.

In some embodiments, about 400 μg to about 4000 μg, about 1000 μg to about 4000 μg, about 1000 μg to about 3000 μg, about 1000 μg to about 2000 μg, about 2000 μg to about 4000 μg, or about 2000 to about 3000 μg of the vitamin is present in the composition. In some cases, about 1600 μg to about 2400 μg, or about 1800 μg to about 2200 μg, or about 1900 μg to about 1100 μg of the vitamin is present in the composition. In some cases, about 1900 μg, about 1950 μg, about 2000 μg, about 2050 μg, or about 2100 μg of the vitamin is present in the composition.

In some embodiments, there are two vitamins (e.g., nicotinamide riboside and methylcobalamin) present in the composition. In some cases, about 200 μg to about 2000 μg, about 500 μg to about 2000 μg, about 500 μg to about 1500 μg, about 500 μg to about 1000 μg, about 1000 μg to about 2000 μg, or about 1000 to about 1500 μg of each vitamin is present in the composition. In some cases, about 800 μg to about 1200 μg, or about 900 μg to about 1100 μg, or about 950 μg to about 1050 μg of each vitamin is present in the composition. In some cases, about 900 μg, about 950 μg, about 1000 μg, about 1050 μg, or about 1100 μg of each vitamin is present in the composition.

In certain embodiments, a composition comprising a GHRH peptide comprises excipients and/or enhancing agents. Excipients, or enhancing agents, may be added to compositions and serve as vehicles for their delivery. An excipient must be stable and reproducible, have no unwished interactions with compositions, be inert and nontoxic. The role of excipients may be to aid in the processing of composition delivery systems during composition manufacture. Excipients may protect, support, or enhance stability and bioavailability and composition delivery. The excipient may be a penetration enhancer. The excipient may include a chelating agent that assists in drug delivery by depleting Ca2+. Non-limiting examples of excipients used in formulations include magnesium stearate, microcrystalline cellulose, starch (corn), silicone/titanium dioxide, stearic acid, sodium starch glycolate, sodium alginate, calcium alginate, gelatin, talc, sucrose, calcium stearate, povodione, pregalantinized starch, HPMC, croscarmellose, hydroxypropyl cellulose, ethycellulose, calcium phosphate, and crospovidone. Alginate is a natural polymer. The use of alginate can provide advantages including ease of preparation, biocompatibility, biodegradability, and nontoxicity. In some embodiments, the alginate is a penetration enhancer. Alginates can be applied to various routes of composition administration including targeted or localized composition-delivery systems. In some embodiments, the excipient comprises an alginate. In further embodiments the excipient comprises sodium alginate. In some embodiments, about 1 μg to about 30 μg, about 1 μg to about 20 μg, about 1 μg to about 15 μg, about 1 μg to about 10 μg, about 5 μg to about 30 μg, about 5 μg to about 20 μg, about 5 μg to about 15 μg, or about 5 to about 10 μg of the excipient is present in the composition. In some cases, about 8 μg to about 15 μg, about 8 μg to about 14 μg, about 8 μg to about 13 μg, about 8 μg to about 12 μg, about 9 μg to about 15 μg, about 9 μg to about 14 μg, about 9 μg to about 13 μg, about 9 μg to about 12 μg, about 10 μg to about 15 μg, about 10 μg to about 14 μg, about 10 μg to about 13 μg, about 10 μg to about 12 μg, or about 11 μg to about 13 μg of the excipient is present in the composition. In some cases, about 8 μg, about 9 μg, about 10 μg, about 11 μg, about 12 μg, about 13 μg, about 14 μg, or about 15 μg of the excipient is present in the composition. In some embodiments, the composition further comprises a delivery-enhancing agent, surfactant, alkyl glycoside, stimulatory agent, neurotransmitter, amino acid, steroid, or vitamin, or two or more thereof.

In certain embodiments a GHRH peptide is formulated in a composition with a delivery-enhancing agent, surfactant, alkyl glycoside, stimulatory agent, neurotransmitter, amino acid, steroid, or vitamin, or two or more thereof.

In some embodiments, the composition comprises a GHRH peptide (e.g., a peptide of Table 1, or a peptide having at least 80%, 81%, 82%, 83%, 84%, 85%, 86%, 87%, 88%, 89%, 90%, 91%, 92%, 93%, 94%, 95%, 96%, 97%, 98%, 99% or 100% identical to a sequence of Table 1) that is present in the composition from about 200 μg to about 2000 μg, about 500 μg to about 2000 μg, about 500 μg to about 1500 μg, about 500 μg to about 1000 μg, about 1000 μg to about 2000 μg, or about 1000 to about 1500 μg; and: (a) a delivery-enhancing agent and/or surfactant that is present in the composition from about 1 μg to about 30 μg, about 1 μg to about 20 μg, about 1 μg to about 15 μg, about 1 μg to about 10 μg, about 5 μg to about 30 μg, about 5 μg to about 20 μg, about 5 μg to about 15 μg, or about 5 to about 10 μg; (b) a stimulatory agent and/or neurotransmitter and/or amino acid that is present in the composition from about 50 μg to about 500 μg, about 100 μg to about 500 μg, about 200 μg to about 500 μg, about 50 μg to about 400 μg, about 50 μg to about 300 μg, about 100 μg to about 300 μg, or about 200 to about 300 μg; (c) a steroid that is present in the composition from about 200 μg to about 2000 μg, about 500 μg to about 2000 μg, about 500 μg to about 1500 μg, about 500 μg to about 1000 μg, about 1000 μg to about 2000 μg, or about 1000 to about 1500 μg; (d) one or more vitamins, wherein each vitamin is present in the composition from about 200 μg to about 2000 μg, about 500 μg to about 2000 μg, about 500 μg to about 1500 μg, about 500 μg to about 1000 μg, about 1000 μg to about 2000 μg, or about 1000 to about 1500 μg; (e) an excipient; (f) a combination of (a) and (b); (g) a combination of (a) and (c); (h) a combination of (a) and (d); (i) a combination of (a) and (e); (j) a combination of (b) and (c); (k) a combination of (b) and (d); (l) a combination of (b) and (e); (m) a combination of (c) and (d); (n) a combination of (c) and (e); (o) a combination of (d) and (e); (p) a combination of (a), (b), and (c); (q) a combination of (a), (b), and (d); (r) a combination of (a), (b), and (e); (s) a combination of (a), (c), and (d); (t) a combination of (a), (c), and (e); (u) a combination of (a), (d), and (e); (v) a combination of (b), (c), and (d); (w) a combination of (b), (c), and (e); (x) a combination of (c), (d), and (e); (y) a combination of (a), (b), (c), and (d); (z) a combination of (a), (b), (c), and (e); or (aa) a combination of (b), (c), (d), and (e). The excipient may be present in the composition from about 1 μg to about 30 μg, about 1 μg to about 20 μg, about 1 μg to about 15 μg, about 1 μg to about 10 μg, about 5 μg to about 30 μg, about 5 μg to about 20 μg, about 5 μg to about 15 μg, or about 5 to about 10 μg.

In some embodiments, the composition comprises a GHRH peptide (e.g., a peptide of Table 1, or a peptide having at least 80%, 81%, 82%, 83%, 84%, 85%, 86%, 87%, 88%, 89%, 90%, 91%, 92%, 93%, 94%, 95%, 96%, 97%, 98%, 99% or 100% identical to a sequence of Table 1) that is present in the composition from about 200 μg to about 2000 μg, about 500 μg to about 2000 μg, about 500 μg to about 1500 μg, about 500 μg to about 1000 μg, about 1000 μg to about 2000 μg, or about 1000 to about 1500 μg; and glycine that is present in the composition from about 50 μg to about 3000 μg, about 50 μg to about 2900 μg, about 50 μg to about 2800 μg, about 50 μg to about 2700 μg, about 50 μg to about 2600 μg, about 50 μg to about 2500 μg, about 50 μg to about 2400 μg, about 50 μg to about 2300 μg, about 50 μg to about 2200 μg, about 50 μg to about 2100 μg, about 50 μg to about 2000 μg, about 50 μg to about 1900 μg, about 50 μg to about 1800 μg, about 50 μg to about 1700 μg, about 50 μg to about 1600 μg, about 50 μg to about 1500 μg, about 50 μg to about 1400 μg, about 50 μg to about 1300 μg, about 50 μg to about 1200 μg, about 50 μg to about 1100 μg, about 50 μg to about 1000 μg, about 50 μg to about 900 μg, about 50 μg to about 800 μg, about 50 μg to about 700 μg, about 50 μg to about 600 μg, about 50 μg to about 500 μg, about 300 μg to about 700 μg, or about 350 μg to about 700 μg, about 400 μg to about 700 μg, about 450 μg to about 700 μg, about 500 μg to about 700 μg, about 300 μg to about 650 μg, about 350 μg to about 650 μg, about 400 μg to about 650 μg, about 450 μg to about 650 μg, about 500 μg to about 650 μg, about 300 μg to about 600 μg, about 350 μg to about 600 μg, about 400 μg to about 600 μg, about 450 μg to about 600 μg, about 500 μg to about 600 μg, about 300 μg to about 550 μg, about 350 μg to about 550 μg, about 400 μg to about 550 μg, about 450 μg to about 550 μg, about 500 μg to about 550 μg, about 300 μg to about 500 μg, about 350 μg to about 500 μg, about 400 μg to about 500 μg, or about 450 μg to about 500 μg, about 800 μg to about 1200 μg, or about 850 μg to about 1200 μg, about 900 μg to about 1200 μg, about 950 μg to about 1200 μg, about 1000 μg to about 1200 μg, about 800 μg to about 1150 μg, about 850 μg to about 1150 μg, about 900 μg to about 1150 μg, about 950 μg to about 1150 μg, about 1000 μg to about 1150 μg, about 800 μg to about 1100 μg, about 850 μg to about 1100 μg, about 900 μg to about 1100 μg, about 950 μg to about 1100 μg, about 1000 μg to about 1100 μg, about 800 μg to about 1050 μg, about 850 μg to about 1050 μg, about 900 μg to about 1050 μg, about 950 μg to about 1050 μg, about 1000 μg to about 1050 μg, about 800 μg to about 1000 μg, about 850 μg to about 1000 μg, about 900 μg to about 1000 μg, or about 950 μg to about 1000 μg, about 1300 μg to about 1700 μg, or about 1350 μg to about 1700 μg, about 1400 μg to about 1700 μg, about 1450 μg to about 1700 μg, about 1500 μg to about 1700 μg, about 1300 μg to about 1650 μg, about 1350 μg to about 1650 μg, about 1400 μg to about 1650 μg, about 1450 μg to about 1650 μg, about 1500 μg to about 1650 μg, about 1300 μg to about 1600 μg, about 1350 μg to about 1600 μg, about 1400 μg to about 1600 μg, about 1450 μg to about 1600 μg, about 1500 μg to about 1600 μg, about 1300 μg to about 1550 μg, about 1350 μg to about 1550 μg, about 1400 μg to about 1550 μg, about 1450 μg to about 1550 μg, about 1500 μg to about 1550 μg, about 1300 μg to about 1500 μg, about 1350 μg to about 1500 μg, about 1400 μg to about 1500 μg, or about 1450 μg to about 1500 μg, about 1800 μg to about 2200 μg, or about 1850 μg to about 2200 μg, about 1900 μg to about 2200 μg, about 1950 μg to about 2200 μg, about 2000 μg to about 2200 μg, about 1800 μg to about 2150 μg, about 1850 μg to about 2150 μg, about 1900 μg to about 2150 μg, about 1950 μg to about 2150 μg, about 2000 μg to about 2150 μg, about 1800 μg to about 2100 μg, about 1850 μg to about 2100 μg, about 1900 μg to about 2100 μg, about 1950 μg to about 2100 μg, about 2000 μg to about 2100 μg, about 1800 μg to about 2050 μg, about 1850 μg to about 2050 μg, about 1900 μg to about 2050 μg, about 1950 μg to about 2050 μg, about 2000 μg to about 2050 μg, about 1800 μg to about 2000 μg, about 1850 μg to about 2000 μg, about 1900 μg to about 2000 μg, or about 1950 μg to about 2000 μg, about 2300 μg to about 2700 μg, or about 2350 μg to about 2700 μg, about 2400 μg to about 2700 μg, about 2450 μg to about 2700 μg, about 2500 μg to about 2700 μg, 2300 μg to about 2650 μg, about 2350 μg to about 2650 μg, about 2400 μg to about 2650 μg, about 2450 μg to about 2650 μg, about 2500 μg to about 2650 μg, about 2300 μg to about 2600 μg, about 2350 μg to about 2600 μg, about 2400 μg to about 2600 μg, about 2450 μg to about 2600 μg, about 2500 μg to about 2600 μg, about 2300 μg to about 2550 μg, about 2350 μg to about 2550 μg, about 2400 μg to about 2550 μg, about 2450 μg to about 2550 μg, about 2500 μg to about 2550 μg, 2300 μg to about 2500 μg, about 2350 μg to about 2500 μg, about 2400 μg to about 2500 μg, or about 2450 μg to about 2500 μg, about 50 μg to about 500 μg, about 100 μg to about 500 μg, about 200 μg to about 500 μg, about 50 μg to about 400 μg, about 50 μg to about 300 μg, about 100 μg to about 300 μg, or about 200 to about 300 μg. In some embodiments, about 400 ug, about 410 ug, about 420 ug, about 430 ug, about 440 ug, about 450 ug, about 460 ug, about 470 ug, about 480 ug, about 490 ug, about 500 ug, about 510 ug, about 520 ug, about 530 ug, about 540 ug, about 550 ug, about 560 ug, about 570 ug, about 580 ug, about 590 ug, or about 600 ug of glycine is present in the composition. In some embodiments, about 500 ug of glycine is present in the composition. In some embodiments, about 900 ug, about 910 ug, about 920 ug, about 930 ug, about 940 ug, about 950 ug, about 960 ug, about 970 ug, about 980 ug, about 990 ug, about 1000 ug, about 1010 ug, about 1020 ug, about 1030 ug, about 1040 ug, about 1050 ug, about 1060 ug, about 1070 ug, about 1080 ug, about 1090 ug, or about 1100 ug of glycine is present in the composition. In some embodiments, about 1000 ug of glycine is present in the composition. In some embodiments, about 1400 ug, about 1410 ug, about 1420 ug, about 1430 ug, about 1440 ug, about 1450 ug, about 1460 ug, about 1470 ug, about 1480 ug, about 1490 ug, about 1500 ug, about 1510 ug, about 1520 ug, about 1530 ug, about 1540 ug, about 1550 ug, about 1560 ug, about 1570 ug, about 1580 ug, about 1590 ug, or about 1600 ug of glycine is present in the composition. In some embodiments, about 1500 ug of glycine is present in the composition. In some embodiments, about 1900 ug, about 1910 ug, about 1920 ug, about 1930 ug, about 1940 ug, about 1950 ug, about 1960 ug, about 1970 ug, about 1980 ug, about 1990 ug, about 2000 ug, about 2010 ug, about 2020 ug, about 2030 ug, about 2040 ug, about 2050 ug, about 2060 ug, about 2070 ug, about 2080 ug, about 2090 ug, or about 2100 ug of glycine is present in the composition. In some embodiments, about 2000 ug of glycine is present in the composition. In some embodiments, about 2400 ug, about 2410 ug, about 2420 ug, about 2430 ug, about 2440 ug, about 2450 ug, about 2460 ug, about 2470 ug, about 2480 ug, about 2490 ug, about 2500 ug, about 2510 ug, about 2520 ug, about 2530 ug, about 2540 ug, about 2550 ug, about 2560 ug, about 2570 ug, about 2580 ug, about 2590 ug, or about 2600 ug of glycine is present in the composition. In some embodiments, about 2500 μg of glycine is present in the composition. In some cases, the composition comprises one or more additional agents and/or excipients, e.g., one or more of the following: stimulatory agent, surfactant, neurotransmitter, amino acid, steroid, and vitamin.

In certain embodiments the delivery-enhancing agent and/or surfactant component comprises an alkyl saccharide. Non-limiting examples of saccharides covalently joined to an alkyl include glucose, maltose, maltotriose, maltotetrose, sucrose and trehalose. Non-limiting examples of alkyl glycosides include octyl-, nonyl-, decyl-, undecyl-, dodecyl, tridecyl, tetradecyl, pentadecyl, octadecyl a- or β-D-maltoside,-giucoside or sucroside. Non-limiting embodiments include alkyl glycosides marketed under the name Intravail. In certain embodiments the stimulatory agent and/or neurotransmitter component comprises neurotransmitters that play a stimulatory role in growth hormone secretion. Non-limiting examples of such stimulatory agents and/or neurotransmitters include choline, arginine, ornithin, glycine, lysine, glutamine and niacin. In certain embodiments the steroid component comprises a steroidal glycoside. Non-limiting examples of steroidal glycosides include ginsenoside saponins. Non-limiting examples of ginsenoside saponins are ginsenosides: Rb1, Rb2, Rc, Rd, Re, Rg1, and Rg3. In certain embodiments the vitamin comprises one or more vitamins or vitamin analogs. Non-limiting examples of vitamins or vitamin analogs include vitamin (e.g., vitamin B3, vitamin B12) pyridoxine, cyanocobalamin, ascorbic acid, biotin niacin, folate, pantothenic acid, riboflavin, thiamine, choline, carnitine, folic acid, adenosylcobalamin, hydroxycobalamin, nicotinamide riboside, nicotinamide adenine dinucleotide, nicotinamide, methylcobalamin, and nicotinic acid. In certain embodiments the excipient comprises a non-toxic, stable excipient for composition formulation. Non-limiting examples of excipients include magnesium stearate, microcrystalline cellulose, starch (corn), silicone/titanium dioxide, stearic acid, sodium starch glycolate, sodium alginate, calcium alginate, gelatin, talc, sucrose, calcium stearate, povodione, pregalantinized starch, HPMC, croscarmellose, hydroxypropyl cellulose, ethycellulose, calcium phosphate, and crospovidone. In some embodiments the excipient comprises an alginate. In some embodiments, the alginate is a penetration enhancer.

In some embodiments, the composition has an active ingredient weight of about 0.1 mg to about 8 mg, about 0.5 mg to about 7 mg, about 2 mg to about 6 mg, or about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 2.1 mg, about 2.2 mg, about 2.3 mg, about 2.4 mg, about 2.5 mg, about 2.6 mg, about 2.7 mg, about 2.8 mg, about 2.9 mg, about 3 mg, about 3.1 mg, about 3.2 mg, about 3.3 mg, about 3.4 mg, about 3.5 mg, about 3.6 mg, about 3.7 mg, about 3.8 mg, about 3.9 mg, about 4 mg, about 4.1 mg, about 4.2 mg, about 4.3 mg, about 4.4 mg, about 4.5 mg, about 4.6 mg, about 4.7 mg, about 4.8 mg, about 4.9 mg, about 5 mg, about 5.1 mg, about 5.2 mg, about 5.3 mg, about 5.4 mg, about 5.5 mg, about 5.6 mg, about 5.7 mg, about 5.8 mg, about 5.9 mg, about 6 mg, about 6.1 mg, about 6.2 mg, about 6.3 mg, about 6.4 mg, about 6.5 mg, about 6.6 mg, about 6.7 mg, about 6.8 mg, about 6.9 mg, about 7 mg, about 7.1 mg, about 7.2 mg, about 7.3 mg, about 7.4 mg, about 7.5 mg, about 7.6 mg, about 7.7 mg, about 7.8 mg, about 7.9 mg, or about 8 mg. In some embodiments, the composition has an active ingredient weight of about 1 mg to about 5 mg, about 1 mg to about 4.9 mg, about 1 mg to about 4.8 mg, about 1 mg to about 4.7 mg, about 1 mg to about 4.6 mg, about 1 mg to about 4.5 mg, about 1 mg to about 4.4 mg, about 1 mg to about 4.3 mg, about 1 mg to about 4.2 mg, about 1 mg to about 4.1 mg, about 1 mg to about 4 mg, about 2 mg to about 5 mg, about 2 mg to about 4.9 mg, about 2 mg to about 4.8 mg, about 2 mg to about 4.7 mg, about 2 mg to about 4.6 mg, about 2 mg to about 4.5 mg, about 2 mg to about 4.4 mg, about 2 mg to about 4.3 mg, about 2 mg to about 4.2 mg, about 2 mg to about 4.1 mg, about 2 mg to about 4 mg, about 1 mg to about 4 mg, about 1 mg to about 3.9 mg, about 1 mg to about 3.8 mg, about 1 mg to about 3.7 mg, about 1 mg to about 3.6 mg, about 1 mg to about 3.5 mg, about 1 mg to about 3.4 mg, about 1 mg to about 3.3 mg, about 1 mg to about 3.2 mg, about 1 mg to about 3.1 mg, about 1 mg to about 3 mg, about 2 mg to about 4 mg, about 2 mg to about 3.9 mg, about 2 mg to about 3.8 mg, about 2 mg to about 3.7 mg, about 2 mg to about 3.6 mg, about 2 mg to about 3.5 mg, about 2 mg to about 3.4 mg, about 2 mg to about 3.3 mg, about 2 mg to about 3.2 mg, about 2 mg to about 3.1 mg, or about 2 mg to about 3 mg. For instance, the composition has an active ingredient weight of about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1 mg, about 1.05 mg, about 1.1 mg, about 1.15 mg, about 1.2 mg, about 1.25 mg, about 1.3 mg, about 1.35 mg, about 1.4 mg, about 1.45 mg, or about 1.5 mg. In some embodiments, the composition has an active ingredient weight of about 0.5 mg to about 2 mg, about 0.5 mg to about 1.9 mg, about 0.5 mg to about 1.8 mg, about 0.5 mg to about 1.7 mg, about 0.5 mg to about 1.6 mg, about 0.5 mg to about 1.5 mg, about 0.5 mg to about 1.4 mg, about 0.5 mg to about 1.3 mg, about 0.5 mg to about 1.2 mg, about 0.5 mg to about 1.1 mg, about 0.5 mg to about 1 mg, about 0.6 mg to about 2 mg, about 0.6 mg to about 1.9 mg, about 0.6 mg to about 1.8 mg, about 0.6 mg to about 1.7 mg, about 0.6 mg to about 1.6 mg, about 0.6 mg to about 1.5 mg, about 0.6 mg to about 1.4 mg, about 0.6 mg to about 1.3 mg, about 0.6 mg to about 1.2 mg, about 0.6 mg to about 1.1 mg, about 0.6 mg to about 1 mg, about 0.7 mg to about 2 mg, about 0.7 mg to about 1.9 mg, about 0.7 mg to about 1.8 mg, about 0.7 mg to about 1.7 mg, about 0.7 mg to about 1.6 mg, about 0.7 mg to about 1.5 mg, about 0.7 mg to about 1.4 mg, about 0.7 mg to about 1.3 mg, about 0.7 mg to about 1.2 mg, about 0.7 mg to about 1.1 mg, about 0.7 mg to about 1 mg, about 0.8 mg to about 2 mg, about 0.8 mg to about 1.9 mg, about 0.8 mg to about 1.8 mg, about 0.8 mg to about 1.7 mg, about 0.8 mg to about 1.6 mg, about 0.8 mg to about 1.5 mg, about 0.8 mg to about 1.4 mg, about 0.8 mg to about 1.3 mg, about 0.8 mg to about 1.2 mg, about 0.8 mg to about 1.1 mg, about 0.8 mg to about 1 mg, about 0.9 mg to about 2 mg, about 0.9 mg to about 1.9 mg, about 0.9 mg to about 1.8 mg, about 0.9 mg to about 1.7 mg, about 0.9 mg to about 1.6 mg, about 0.9 mg to about 1.5 mg, about 0.9 mg to about 1.4 mg, about 0.9 mg to about 1.3 mg, about 0.9 mg to about 1.2 mg, about 0.9 mg to about 1.1 mg, or about 0.9 mg to about 1 mg. For instance, the composition has an active ingredient weight of about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1 mg, about 1.05 mg, about 1.1 mg, about 1.15 mg, about 1.2 mg, about 1.25 mg, about 1.3 mg, about 1.35 mg, about 1.4 mg, about 1.45 mg, or about 1.5 mg. In a non-limiting example, active ingredients include GHRH peptide and optionally glycine.

In some embodiments, the composition has about 200 μg to about 2000 μg of GHRH peptide, e.g., about 200 μg to about 1800 μg, about 200 μg to about 1600 μg, about 200 μg to about 1400 μg, about 200 μg to about 1200 μg, about 200 μg to about 1000 μg, about 200 μg to about 800 μg, about 400 μg to about 2000 μg, about 400 μg to about 1800 μg, about 400 μg to about 1600 μg, about 400 μg to about 1400 μg, about 400 μg to about 1200 μg, about 400 μg to about 1000 μg, about 400 μg to about 800 μg, about 600 μg to about 2000 μg, about 600 μg to about 1800 μg, about 600 μg to about 1600 μg, about 600 μg to about 1400 μg, about 600 μg to about 1200 μg, about 600 μg to about 1000 μg, about 600 μg to about 800 μg, about 800 μg to about 2000 μg, about 800 μg to about 1800 μg, about 800 μg to about 1600 μg, about 800 μg to about 1400 μg, about 800 μg to about 1200 μg, about 800 μg to about 1000 μg, about 800 μg, about 850 μg, about 900 μg, about 950 μg, about 1000 μg, about 1050 μg, about 1100 μg, about 1150 μg, about 1200 μg, about 1250 μg, about 1300 μg, about 1400 μg, about 1500 μg, about 1600 μg, about 1800 μg, or about 2000 μg of GHRH peptide (e.g., a peptide comprising a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1 (YADAIFTNSY RKVLGQLSAR KLLQDIMSR), SEQ ID NO: 2 (YADAIFTNSY RKVLGQLSAR KLLQDIMSR-NH2), or a sequence of Table 1). In some embodiments, the composition has about 50 μg to about 3000 μg or about 50 μg to about 500 μg of stimulatory agent and/or neurotransmitter and/or amino acid (e.g., glycine), e.g., about 50 μg to about 3000 μg, about 50 μg to about 2900 μg, about 50 μg to about 2800 μg, about 50 μg to about 2700 μg, about 50 μg to about 2600 μg, about 50 μg to about 2500 μg, about 50 μg to about 2400 μg, about 50 μg to about 2300 μg, about 50 μg to about 2200 μg, about 50 μg to about 2100 μg, about 50 μg to about 2000 μg, about 50 μg to about 1900 μg, about 50 μg to about 1800 μg, about 50 μg to about 1700 μg, about 50 μg to about 1600 μg, about 50 μg to about 1500 μg, about 50 μg to about 1400 μg, about 50 μg to about 1300 μg, about 50 μg to about 1200 μg, about 50 μg to about 1100 μg, about 50 μg to about 1000 μg, about 50 μg to about 900 μg, about 50 μg to about 800 μg, about 50 μg to about 700 μg, about 50 μg to about 600 μg, about 100 μg to about 3000 μg, about 200 μg to about 3000 μg, about 300 μg to about 3000 μg, about 400 μg to about 3000 μg, about 500 μg to about 3000 μg, about 600 μg to about 3000 μg, about 700 μg to about 3000 μg, about 800 μg to about 3000 μg, about 900 μg to about 3000 μg, about 1000 μg to about 3000 μg, about 1100 μg to about 3000 μg, about 1200 μg to about 3000 μg, about 1300 μg to about 3000 μg, about 1400 μg to about 3000 μg, about 1500 μg to about 3000 μg, about 1600 μg to about 3000 μg, about 1700 μg to about 3000 μg, about 1800 μg to about 3000 μg, about 1900 μg to about 3000 μg, about 2000 μg to about 3000 μg, about 2100 μg to about 3000 μg, about 2200 μg to about 3000 μg, about 2300 μg to about 3000 μg, about 2400 μg to about 3000 μg, about 2500 μg to about 3000 μg, about 2600 μg to about 3000 μg, about 2700 μg to about 3000 μg, about 2800 μg to about 3000 μg, about 2900 μg to about 3000 μg, about 100 μg to about 2500 μg, about 200 μg to about 2500 μg, about 300 μg to about 2500 μg, about 400 μg to about 2500 μg, about 500 μg to about 2500 μg, about 600 μg to about 2500 μg, about 700 μg to about 2500 μg, about 800 μg to about 2500 μg, about 900 μg to about 2500 μg, about 1000 μg to about 2500 μg, about 1100 μg to about 2500 μg, about 1200 μg to about 2500 μg, about 1300 μg to about 2500 μg, about 1400 μg to about 2500 μg, about 1500 μg to about 2500 μg, about 1600 μg to about 2500 μg, about 1700 μg to about 2500 μg, about 1800 μg to about 2500 μg, about 1900 μg to about 2500 μg, about 2000 μg to about 2500 μg, about 2100 μg to about 2500 μg, about 2200 μg to about 2500 μg, about 2300 μg to about 2500 μg, about 2400 μg to about 2500 μg, about 100 μg to about 2000 μg, about 200 μg to about 2000 μg, about 300 μg to about 2000 μg, about 400 μg to about 2000 μg, about 500 μg to about 2000 μg, about 600 μg to about 2000 μg, about 700 μg to about 2000 μg, about 800 μg to about 2000 μg, about 900 μg to about 2000 μg, about 1000 μg to about 2000 μg, about 1100 μg to about 2000 μg, about 1200 μg to about 2000 μg, about 1300 μg to about 2000 μg, about 1400 μg to about 2000 μg, about 1500 μg to about 2000 μg, about 1600 μg to about 2000 μg, about 1700 μg to about 2000 μg, about 1800 μg to about 2000 μg, about 1900 μg to about 2000 μg, about 100 μg to about 1500 μg, about 200 μg to about 1500 μg, about 300 μg to about 1500 μg, about 400 μg to about 1500 μg, about 500 μg to about 1500 μg, about 600 μg to about 1500 μg, about 700 μg to about 1500 μg, about 800 μg to about 1500 μg, about 900 μg to about 1500 μg, about 1000 μg to about 1500 μg, about 1100 μg to about 1500 μg, about 1200 μg to about 1500 μg, about 1300 μg to about 1500 μg, about 1400 μg to about 1500 μg, about 100 μg to about 1000 μg, about 200 μg to about 1000 μg, about 300 μg to about 1000 μg, about 400 μg to about 1000 μg, about 500 μg to about 1000 μg, about 600 μg to about 1000 μg, about 700 μg to about 1000 μg, about 800 μg to about 1000 μg, about 900 μg to about 1000 μg, about 50 μg to about 500 μg, about 50 μg to about 450 μg, about 50 μg to about 400 μg, about 50 μg to about 350 μg, about 50 μg to about 300 μg, about 50 μg to about 250 μg, about 50 μg to about 200 μg, about 50 μg to about 150 μg, about 50 μg to about 100 μg, about 100 μg to about 500 μg, about 100 μg to about 450 μg, about 100 μg to about 400 μg, about 100 μg to about 350 μg, about 100 μg to about 300 μg, about 100 μg to about 250 μg, about 100 μg to about 200 μg, about 100 μg to about 150 μg, about 150 μg to about 500 μg, about 150 μg to about 450 μg, about 150 μg to about 400 μg, about 150 μg to about 350 μg, about 150 μg to about 300 μg, about 150 μg to about 250 μg, about 150 μg to about 200 μg, about 200 μg to about 500 μg, about 200 μg to about 450 μg, about 200 μg to about 400 μg, about 200 μg to about 350 μg, about 200 μg to about 300 μg, about 200 μg to about 250 μg, about 250 μg to about 500 μg, about 250 μg to about 450 μg, about 250 μg to about 400 μg, about 250 μg to about 350 μg, about 250 μg to about 300 μg, about 50 μg, about 100 μg, about 150 μg, about 200 μg, about 300 μg to about 700 μg, or about 350 μg to about 700 μg, about 400 μg to about 700 μg, about 450 μg to about 700 μg, about 500 μg to about 700 μg, about 300 μg to about 650 μg, about 350 μg to about 650 μg, about 400 μg to about 650 μg, about 450 μg to about 650 μg, about 500 μg to about 650 μg, about 300 μg to about 600 μg, about 350 μg to about 600 μg, about 400 μg to about 600 μg, about 450 μg to about 600 μg, about 500 μg to about 600 μg, about 300 μg to about 550 μg, about 350 μg to about 550 μg, about 400 μg to about 550 μg, about 450 μg to about 550 μg, about 500 μg to about 550 μg, about 300 μg to about 500 μg, about 350 μg to about 500 μg, about 400 μg to about 500 μg, or about 450 μg to about 500 μg of glycine is present in the composition. In some embodiments, about 800 μg to about 1200 μg, or about 850 μg to about 1200 μg, about 900 μg to about 1200 μg, about 950 μg to about 1200 μg, about 1000 μg to about 1200 μg, about 800 μg to about 1150 μg, about 850 μg to about 1150 μg, about 900 μg to about 1150 μg, about 950 μg to about 1150 μg, about 1000 μg to about 1150 μg, about 800 μg to about 1100 μg, about 850 μg to about 1100 μg, about 900 μg to about 1100 μg, about 950 μg to about 1100 μg, about 1000 μg to about 1100 μg, about 800 μg to about 1050 μg, about 850 μg to about 1050 μg, about 900 μg to about 1050 μg, about 950 μg to about 1050 μg, about 1000 μg to about 1050 μg, about 800 μg to about 1000 μg, about 850 μg to about 1000 μg, about 900 μg to about 1000 μg, or about 950 μg to about 1000 μg of glycine is present in the composition. In some embodiments, about 1300 μg to about 1700 μg, or about 1350 μg to about 1700 μg, about 1400 μg to about 1700 μg, about 1450 μg to about 1700 μg, about 1500 μg to about 1700 μg, about 1300 μg to about 1650 μg, about 1350 μg to about 1650 μg, about 1400 μg to about 1650 μg, about 1450 μg to about 1650 μg, about 1500 μg to about 1650 μg, about 1300 μg to about 1600 μg, about 1350 μg to about 1600 μg, about 1400 μg to about 1600 μg, about 1450 μg to about 1600 μg, about 1500 μg to about 1600 μg, about 1300 μg to about 1550 μg, about 1350 μg to about 1550 μg, about 1400 μg to about 1550 μg, about 1450 μg to about 1550 μg, about 1500 μg to about 1550 μg, about 1300 μg to about 1500 μg, about 1350 μg to about 1500 μg, about 1400 μg to about 1500 μg, or about 1450 μg to about 1500 μg of glycine is present in the composition. In some embodiments, about 1800 μg to about 2200 μg, or about 1850 μg to about 2200 μg, about 1900 μg to about 2200 μg, about 1950 μg to about 2200 μg, about 2000 μg to about 2200 μg, about 1800 μg to about 2150 μg, about 1850 μg to about 2150 μg, about 1900 μg to about 2150 μg, about 1950 μg to about 2150 μg, about 2000 μg to about 2150 μg, about 1800 μg to about 2100 μg, about 1850 μg to about 2100 μg, about 1900 μg to about 2100 μg, about 1950 μg to about 2100 μg, about 2000 μg to about 2100 μg, about 1800 μg to about 2050 μg, about 1850 μg to about 2050 μg, about 1900 μg to about 2050 μg, about 1950 μg to about 2050 μg, about 2000 μg to about 2050 μg, about 1800 μg to about 2000 μg, about 1850 μg to about 2000 μg, about 1900 μg to about 2000 μg, or about 1950 μg to about 2000 μg of glycine is present in the composition. In some embodiments, about 2300 μg to about 2700 μg, or about 2350 μg to about 2700 μg, about 2400 μg to about 2700 μg, about 2450 μg to about 2700 μg, about 2500 μg to about 2700 μg, 2300 μg to about 2650 μg, about 2350 μg to about 2650 μg, about 2400 μg to about 2650 μg, about 2450 μg to about 2650 μg, about 2500 μg to about 2650 μg, about 2300 μg to about 2600 μg, about 2350 μg to about 2600 μg, about 2400 μg to about 2600 μg, about 2450 μg to about 2600 μg, about 2500 μg to about 2600 μg, about 2300 μg to about 2550 μg, about 2350 μg to about 2550 μg, about 2400 μg to about 2550 μg, about 2450 μg to about 2550 μg, about 2500 μg to about 2550 μg, 2300 μg to about 2500 μg, about 2350 μg to about 2500 μg, about 2400 μg to about 2500 μg, or about 2450 μg to about 2500 μg, about 400 ug, about 410 ug, about 420 ug, about 430 ug, about 440 ug, about 450 ug, about 460 ug, about 470 ug, about 480 ug, about 490 ug, about 500 ug, about 510 ug, about 520 ug, about 530 ug, about 540 ug, about 550 ug, about 560 ug, about 570 ug, about 580 ug, about 590 ug, about 600 ug, about 900 ug, about 910 ug, about 920 ug, about 930 ug, about 940 ug, about 950 ug, about 960 ug, about 970 ug, about 980 ug, about 990 ug, about 1000 ug, about 1010 ug, about 1020 ug, about 1030 ug, about 1040 ug, about 1050 ug, about 1060 ug, about 1070 ug, about 1080 ug, about 1090 ug, about 1100 ug, about 1400 ug, about 1410 ug, about 1420 ug, about 1430 ug, about 1440 ug, about 1450 ug, about 1460 ug, about 1470 ug, about 1480 ug, about 1490 ug, about 1500 ug, about 1510 ug, about 1520 ug, about 1530 ug, about 1540 ug, about 1550 ug, about 1560 ug, about 1570 ug, about 1580 ug, about 1590 ug, about 1600 ug, about 1900 ug, about 1910 ug, about 1920 ug, about 1930 ug, about 1940 ug, about 1950 ug, about 1960 ug, about 1970 ug, about 1980 ug, about 1990 ug, about 2000 ug, about 2010 ug, about 2020 ug, about 2030 ug, about 2040 ug, about 2050 ug, about 2060 ug, about 2070 ug, about 2080 ug, about 2090 ug, about 2100 ug, about 2400 ug, about 2410 ug, about 2420 ug, about 2430 ug, about 2440 ug, about 2450 ug, about 2460 ug, about 2470 ug, about 2480 ug, about 2490 ug, about 2500 ug, about 2510 ug, about 2520 ug, about 2530 ug, about 2540 ug, about 2550 ug, about 2560 ug, about 2570 ug, about 2580 ug, about 2590 ug, about 2600 ug, about 250 μg, about 300 μg, about 350 μg, about 400 μg, about 450 μg, or about 500 μg of stimulatory agent and/or neurotransmitter and/or amino acid (e.g., glycine). In some embodiments, the composition has about 200 μg to about 2000 μg steroid (e.g., ginsenoside such as ginsenoside Rg3), e.g., about 200 μg to about 1800 μg, about 200 μg to about 1600 μg, about 200 μg to about 1400 μg, about 200 μg to about 1200 μg, about 200 μg to about 1000 μg, about 200 μg to about 800 μg, about 400 μg to about 2000 μg, about 400 μg to about 1800 μg, about 400 μg to about 1600 μg, about 400 μg to about 1400 μg, about 400 μg to about 1200 μg, about 400 μg to about 1000 μg, about 400 μg to about 800 μg, about 600 μg to about 2000 μg, about 600 μg to about 1800 μg, about 600 μg to about 1600 μg, about 600 μg to about 1400 μg, about 600 μg to about 1200 μg, about 600 μg to about 1000 μg, about 600 μg to about 800 μg, about 800 μg to about 2000 μg, about 800 μg to about 1800 μg, about 800 μg to about 1600 μg, about 800 μg to about 1400 μg, about 800 μg to about 1200 μg, about 800 μg to about 1000 μg, about 800 μg, about 850 μg, about 900 μg, about 950 μg, about 1000 μg, about 1050 μg, about 1100 μg, about 1150 μg, about 1200 μg, about 1250 μg, about 1300 μg, about 1400 μg, about 1500 μg, about 1600 μg, about 1800 μg, or about 2000 μg of steroid (e.g., ginsenoside such as ginsenoside Rg3). In some embodiments, the composition has about 200 μg to about 2000 μg vitamin (e.g., nicotinamide riboside and/or methylcobalamin), e.g., about 200 μg to about 1800 μg, about 200 μg to about 1600 μg, about 200 μg to about 1400 μg, about 200 μg to about 1200 μg, about 200 μg to about 1000 μg, about 200 μg to about 800 μg, about 400 μg to about 2000 μg, about 400 μg to about 1800 μg, about 400 μg to about 1600 μg, about 400 μg to about 1400 μg, about 400 μg to about 1200 μg, about 400 μg to about 1000 μg, about 400 μg to about 800 μg, about 600 μg to about 2000 μg, about 600 μg to about 1800 μg, about 600 μg to about 1600 μg, about 600 μg to about 1400 μg, about 600 μg to about 1200 μg, about 600 μg to about 1000 μg, about 600 μg to about 800 μg, about 800 μg to about 2000 μg, about 800 μg to about 1800 μg, about 800 μg to about 1600 μg, about 800 μg to about 1400 μg, about 800 μg to about 1200 μg, about 800 μg to about 1000 μg, about 800 μg, about 850 μg, about 900 μg, about 950 μg, about 1000 μg, about 1050 μg, about 1100 μg, about 1150 μg, about 1200 μg, about 1250 μg, about 1300 μg, about 1400 μg, about 1500 μg, about 1600 μg, about 1800 μg, or about 2000 μg of vitamin (e.g., nicotinamide riboside and/or methylcobalamin). In some cases, the composition comprises two vitamins, e.g., nicotinamide riboside and methylcobalamin, wherein each vitamin is present in the composition at about 200 μg to about 2000 μg, about 200 μg to about 1800 μg, about 200 μg to about 1600 μg, about 200 μg to about 1400 μg, about 200 μg to about 1200 μg, about 200 μg to about 1000 μg, about 200 μg to about 800 μg, about 400 μg to about 2000 μg, about 400 μg to about 1800 μg, about 400 μg to about 1600 μg, about 400 μg to about 1400 μg, about 400 μg to about 1200 μg, about 400 μg to about 1000 μg, about 400 μg to about 800 μg, about 600 μg to about 2000 μg, about 600 μg to about 1800 μg, about 600 μg to about 1600 μg, about 600 μg to about 1400 μg, about 600 μg to about 1200 μg, about 600 μg to about 1000 μg, about 600 μg to about 800 μg, about 800 μg to about 2000 μg, about 800 μg to about 1800 μg, about 800 μg to about 1600 μg, about 800 μg to about 1400 μg, about 800 μg to about 1200 μg, about 800 μg to about 1000 μg, about 800 μg, about 850 μg, about 900 μg, about 950 μg, about 1000 μg, about 1050 μg, about 1100 μg, about 1150 μg, about 1200 μg, about 1250 μg, about 1300 μg, about 1400 μg, about 1500 μg, about 1600 μg, about 1800 μg, or about 2000 μg. In some embodiments, the composition comprises about 1 μg to about 30 μg of excipient (e.g., alginate, such as sodium alginate), e.g., about 1 μg to about 30 μg, about 1 μg to about 29 μg, about 1 μg to about 28 μg, about 1 μg to about 27 μg, about 1 μg to about 26 μg, about 1 μg to about 25 μg, about 1 μg to about 24 μg, about 1 μg to about 23 μg, about 1 μg to about 22 μg, about 1 μg to about 21 μg, about 1 μg to about 20 μg, about 1 μg to about 19 μg, about 1 μg to about 18 μg, about 1 μg to about 17 μg, about 1 μg to about 16 μg, about 1 μg to about 15 μg, about 1 μg to about 14 μg, about 1 μg to about 13 μg, about 1 μg to about 12 μg, about 1 μg to about 11 μg, about 1 μg to about 10 μg, about 1 μg to about 9 μg, about 1 μg to about 8 μg, about 1 μg to about 7 μg, about 1 μg to about 6 μg, about 1 μg to about 5 μg, about 2 μg to about 30 μg, about 2 μg to about 29 μg, about 2 μg to about 28 μg, about 2 μg to about 27 μg, about 2 μg to about 26 μg, about 2 μg to about 25 μg, about 2 μg to about 24 μg, about 2 μg to about 23 μg, about 2 μg to about 22 μg, about 2 μg to about 21 μg, about 2 μg to about 20 μg, about 2 μg to about 19 μg, about 2 μg to about 18 μg, about 2 μg to about 17 μg, about 2 μg to about 16 μg, about 2 μg to about 15 μg, about 2 μg to about 14 μg, about 2 μg to about 13 μg, about 2 μg to about 12 μg, about 2 μg to about 11 μg, about 2 μg to about 10 μg, about 2 μg to about 9 μg, about 2 μg to about 8 μg, about 2 μg to about 7 μg, about 2 μg to about 6 μg, about 2 μg to about 5 μg, about 3 μg to about 30 μg, about 3 μg to about 29 μg, about 3 μg to about 28 μg, about 3 μg to about 27 μg, about 3 μg to about 26 μg, about 3 μg to about 25 μg, about 3 μg to about 24 μg, about 3 μg to about 23 μg, about 3 μg to about 22 μg, about 3 μg to about 21 μg, about 3 μg to about 20 μg, about 3 μg to about 19 μg, about 3 μg to about 18 μg, about 3 μg to about 17 μg, about 3 μg to about 16 μg, about 3 μg to about 15 μg, about 3 μg to about 14 μg, about 3 μg to about 13 μg, about 3 μg to about 12 μg, about 3 μg to about 11 μg, about 3 μg to about 10 μg, about 3 μg to about 9 μg, about 3 μg to about 8 μg, about 3 μg to about 7 μg, about 3 μg to about 6 μg, about 3 μg to about 5 μg, about 4 μg to about 30 μg, about 4 μg to about 29 μg, about 4 μg to about 28 μg, about 4 μg to about 27 μg, about 4 μg to about 26 μg, about 4 μg to about 25 μg, about 4 μg to about 24 μg, about 4 μg to about 23 μg, about 4 μg to about 22 μg, about 4 μg to about 21 μg, about 4 μg to about 20 μg, about 4 μg to about 19 μg, about 4 μg to about 18 μg, about 4 μg to about 17 μg, about 4 μg to about 16 μg, about 4 μg to about 15 μg, about 4 μg to about 14 μg, about 4 μg to about 13 μg, about 4 μg to about 12 μg, about 4 μg to about 11 μg, about 4 μg to about 10 μg, about 4 μg to about 9 μg, about 4 μg to about 8 μg, about 4 μg to about 7 μg, about 4 μg to about 6 μg, about 4 μg to about 5 μg, about 5 μg to about 30 μg, about 5 μg to about 29 μg, about 5 μg to about 28 μg, about 5 μg to about 27 μg, about 5 μg to about 26 μg, about 5 μg to about 25 μg, about 5 μg to about 24 μg, about 5 μg to about 23 μg, about 5 μg to about 22 μg, about 5 μg to about 21 μg, about 5 μg to about 20 μg, about 5 μg to about 19 μg, about 5 μg to about 18 μg, about 5 μg to about 17 μg, about 5 μg to about 16 μg, about 5 μg to about 15 μg, about 5 μg to about 14 μg, about 5 μg to about 13 μg, about 5 μg to about 12 μg, about 5 μg to about 11 μg, about 5 μg to about 10 μg, about 5 μg to about 9 μg, about 5 μg to about 8 μg, about 5 μg to about 7 μg, about 5 μg to about 6 μg, about 10 μg to about 30 μg, about 10 μg to about 29 μg, about 10 μg to about 28 μg, about 10 μg to about 27 μg, about 10 μg to about 26 μg, about 10 μg to about 25 μg, about 10 μg to about 24 μg, about 10 μg to about 23 μg, about 10 μg to about 22 μg, about 10 μg to about 21 μg, about 10 μg to about 20 μg, about 10 μg to about 19 μg, about 10 μg to about 18 μg, about 10 μg to about 17 μg, about 10 μg to about 16 μg, about 10 μg to about 15 μg, about 10 μg to about 14 μg, about 10 μg to about 13 μg, about 10 μg to about 12 μg, about 10 μg to about 11 μg, about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 29 μg, or about 30 μg of excipient (e.g., alginate, such as sodium alginate). In some embodiments, the composition has about 1 μg to about 30 μg of delivery-enhancing agent and/or penetration enhancer and/or surfactant (e.g., alkyl glycoside, such as dodecyl maltoside or DDM), e.g., about 1 μg to about 30 μg, about 1 μg to about 29 μg, about 1 μg to about 28 μg, about 1 μg to about 27 μg, about 1 μg to about 26 μg, about 1 μg to about 25 μg, about 1 μg to about 24 μg, about 1 μg to about 23 μg, about 1 μg to about 22 μg, about 1 μg to about 21 μg, about 1 μg to about 20 μg, about 1 μg to about 19 μg, about 1 μg to about 18 μg, about 1 μg to about 17 μg, about 1 μg to about 16 μg, about 1 μg to about 15 μg, about 1 μg to about 14 μg, about 1 μg to about 13 μg, about 1 μg to about 12 μg, about 1 μg to about 11 μg, about 1 μg to about 10 μg, about 1 μg to about 9 μg, about 1 μg to about 8 μg, about 1 μg to about 7 μg, about 1 μg to about 6 μg, about 1 μg to about 5 μg, about 2 μg to about 30 μg, about 2 μg to about 29 μg, about 2 μg to about 28 μg, about 2 μg to about 27 μg, about 2 μg to about 26 μg, about 2 μg to about 25 μg, about 2 μg to about 24 μg, about 2 μg to about 23 μg, about 2 μg to about 22 μg, about 2 μg to about 21 μg, about 2 μg to about 20 μg, about 2 μg to about 19 μg, about 2 μg to about 18 μg, about 2 μg to about 17 μg, about 2 μg to about 16 μg, about 2 μg to about 15 μg, about 2 μg to about 14 μg, about 2 μg to about 13 μg, about 2 μg to about 12 μg, about 2 μg to about 11 μg, about 2 μg to about 10 μg, about 2 μg to about 9 μg, about 2 μg to about 8 μg, about 2 μg to about 7 μg, about 2 μg to about 6 μg, about 2 μg to about 5 μg, about 3 μg to about 30 μg, about 3 μg to about 29 μg, about 3 μg to about 28 μg, about 3 μg to about 27 μg, about 3 μg to about 26 μg, about 3 μg to about 25 μg, about 3 μg to about 24 μg, about 3 μg to about 23 μg, about 3 μg to about 22 μg, about 3 μg to about 21 μg, about 3 μg to about 20 μg, about 3 μg to about 19 μg, about 3 μg to about 18 μg, about 3 μg to about 17 μg, about 3 μg to about 16 μg, about 3 μg to about 15 μg, about 3 μg to about 14 μg, about 3 μg to about 13 μg, about 3 μg to about 12 μg, about 3 μg to about 11 μg, about 3 μg to about 10 μg, about 3 μg to about 9 μg, about 3 μg to about 8 μg, about 3 μg to about 7 μg, about 3 μg to about 6 μg, about 3 μg to about 5 μg, about 4 μg to about 30 μg, about 4 μg to about 29 μg, about 4 μg to about 28 μg, about 4 μg to about 27 μg, about 4 μg to about 26 μg, about 4 μg to about 25 μg, about 4 μg to about 24 μg, about 4 μg to about 23 μg, about 4 μg to about 22 μg, about 4 μg to about 21 μg, about 4 μg to about 20 μg, about 4 μg to about 19 μg, about 4 μg to about 18 μg, about 4 μg to about 17 μg, about 4 μg to about 16 μg, about 4 μg to about 15 μg, about 4 μg to about 14 μg, about 4 μg to about 13 μg, about 4 μg to about 12 μg, about 4 μg to about 11 μg, about 4 μg to about 10 μg, about 4 μg to about 9 μg, about 4 μg to about 8 μg, about 4 μg to about 7 μg, about 4 μg to about 6 μg, about 4 μg to about 5 μg, about 5 μg to about 30 μg, about 5 μg to about 29 μg, about 5 μg to about 28 μg, about 5 μg to about 27 μg, about 5 μg to about 26 μg, about 5 μg to about 25 μg, about 5 μg to about 24 μg, about 5 μg to about 23 μg, about 5 μg to about 22 μg, about 5 μg to about 21 μg, about 5 μg to about 20 μg, about 5 μg to about 19 μg, about 5 μg to about 18 μg, about 5 μg to about 17 μg, about 5 μg to about 16 μg, about 5 μg to about 15 μg, about 5 μg to about 14 μg, about 5 μg to about 13 μg, about 5 μg to about 12 μg, about 5 μg to about 11 μg, about 5 μg to about 10 μg, about 5 μg to about 9 μg, about 5 μg to about 8 μg, about 5 μg to about 7 μg, about 5 μg to about 6 μg, about 10 μg to about 30 μg, about 10 μg to about 29 μg, about 10 μg to about 28 μg, about 10 μg to about 27 μg, about 10 μg to about 26 μg, about 10 μg to about 25 μg, about 10 μg to about 24 μg, about 10 μg to about 23 μg, about 10 μg to about 22 μg, about 10 μg to about 21 μg, about 10 μg to about 20 μg, about 10 μg to about 19 μg, about 10 μg to about 18 μg, about 10 μg to about 17 μg, about 10 μg to about 16 μg, about 10 μg to about 15 μg, about 10 μg to about 14 μg, about 10 μg to about 13 μg, about 10 μg to about 12 μg, about 10 μg to about 11 μg, about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 29 μg, or about 30 μg of delivery-enhancing agent and/or penetration enhancer and/or surfactant (e.g., alkyl glycoside, such as dodecyl maltoside or DDM.

### Oral Dissolving Table Formulations

In some aspects, further provided herein is an oral disintegrating formulation, e.g., a rapidly disintegrating formulation of a composition herein. The formulation may be in a solid formation, such as a tablet. The formulation may comprise particles of the active ingredient (e.g., peptide, optionally glycine, delivery-enhancing agent, stimulatory agent, steroid, vitamin) dispersed in a matrix of a water soluble or water dispersible carrier material to form a suspension. In some embodiments, the formulation comprises GHRH. In some embodiments, the formulation comprises glycine. In some embodiments, the formulation comprises a delivery-enhancing agent. Non-limiting examples of a delivery-enhancing agent includes surfactant, alkyl saccharide, glycine, aggregation inhibitory agents; charge-modifying agents; pH control agents; degradative enzyme inhibitory agents; mucolytic or mucus clearing agents; ciliostatic agents; membrane penetration-enhancing agents; modulatory agents of epithelial junction physiology; vasodilator agents; selective transport-enhancing agents; and stabilizing delivery vehicles, carriers, mucoadhesives, support or complex-forming species. Additional mucosal delivery-enhancing agents include, for example, citric acid, sodium citrate, propylene glycol, glycerin, ascorbic acid (e.g., L-ascorbic acid), sodium metabisulfite, ethylenediaminetetraacetic acid (EDTA) disodium, benzalkonium chloride, sodium hydroxide, and mixtures thereof. Delivery-enhancing agents include membrane penetration enhancing agents. Non-limiting examples of a membrane penetration enhancing agents include a surfactant; a bile salt; a phospholipid additive, mixed micelle, liposome, or carrier; an alcohol; an enamine; an NO donor compound; a long-chain amphipathic molecule; a small hydrophobic penetration enhancer; sodium or a salicylic acid derivative; a glycerol ester of acetoacetic acid; a cyclodextrin or beta-cyclodextrin derivative; a medium-chain fatty acid; a chelating agent; an amino acid or salt thereof; an N-acetylamino acid or salt thereof; an enzyme degradative to a selected membrane component; an inhibitor of fatty acid synthesis; an inhibitor of cholesterol synthesis; and any combination thereof.

The carrier material may be any water-soluble or water-dispersible material that is pharmaceutically acceptable, inert to the pharmaceutically active substance and which is capable of forming a rapidly disintegrating network, e.g., disintegrates within 10 seconds or less in the mouth. In one embodiment, the carrier material for use in the present composition comprises gelatin, usually pharmaceutical grade gelatin. In some embodiments, the carrier material comprises, for example, hydrolyzed dextrose, dextran, dextrin, maltodextrin, alginates, hydroxyethyl cellulose, sodium carboxymethyl cellulose, microcrystalline cellulose, corn-syrup solids, pectin, carrageenan, agar, chitosan, locust bean gum, xanthan gum, guar gum, acacia gum, tragacanth, conjac flower, rice flower, wheat gluten, sodium starch glycolate, soy fiber protein, potato protein, papain, horseradish peroxidase, glycine, or mannitol, or any combination of two or more thereof. In some embodiments, the carrier material comprises water. Matrix forming agents suitable for use in dissolving formulations include materials derived from animal or vegetable proteins, such as the gelatins, collagens, dextrins and soy, wheat and psyllium seed proteins; gums such as acacia, guar, agar, and xanthan; polysaccharides; alginates; carrageenans; dextrans; carboxymethylcelluloses; pectins; synthetic polymers such as polyvinylpyrrolidone; and polypeptide/protein or polysaccharide complexes such as gelatin-acacia complexes. In some aspects, gelatin, including fish gelatin or porcine gelatin is used.

In some embodiments, the particles are coated to prevent or minimize early release of the formulation during processing, and/or to prevent early release during rapid disintegration of the dosage form in the mouth. The coated particles may be produced using any of the coating techniques capable of producing particles in the size range of interest. Non-limiting examples include solvent evaporation, solvent extraction, coarcevation, spray congealing, spray drying, pan coating, air suspension techniques, spheronization, lyposomes, complex formation, hot-melt encapsulation, interfacial polymerization, electrostatic encapsulation, ion-exchange resins, centrifugal processes, or combinations thereof. In some embodiments the coating on the particles is a polymer or lipid material and serves to prevent loss of the active ingredient during processing, as well as delaying release of the ingredient beyond the point of disintegration of the form in the mouth. In some aspects, any suitable polymer or lipid or combination can be used as the coating material. Non-limiting examples of suitable polymers include cellulose and cellulose derivatives such as ethylcellulose, hydroxyethylcellulose, hydroxypropylmethylcellulose, hydroxypropylcellulose, methylcellulose, cellulose acetate, cellulose acetate phthalate, hydroxypropylmethylcellulosephthalate, acrylic derivatives, such as polymethacrylates, polyglycolic-polylactic acid, polyvinylalcohol, gelatin, collagen and polyethyleneglycol. Non-limiting examples of suitable lipid materials include waxes such as beeswax and lanolin, stearic acid and derivatives such as glycerol esters, fixed oils, fats, phospholipids, and glycolipids.

In some aspects the continuous phase used for forming the suspension of the active ingredient of the composition is water. The water may be admixed, if desired, with a co-solvent such as an alcohol, e.g. ethanol.

According to the one embodiment of a process, sedimentation in the compound suspension in the carrier material is controlled by manipulation of the matrix temperature to create a more viscous solution. In a nonlimiting example, by cooling a 3% gelatin solution from about 25° C. to about 15° C., the viscosity increases from about 2.0 mPa to 50.0 mPa. By following this approach, it is possible to sufficiently delay the rate of sedimentation of coated particles without significantly altering the physical properties of the finished units.

In certain aspects the suspension of active ingredient(s) and carrier material(s) has a solids content of 50% by weight or less, e.g., 5-25% by weight. In some embodiments, the solids content by weight and the dosing and freeze-drying processes produces a product with a very rapid disintegration time.

In some aspects the suspension may contain other additional ingredients such as, for example, flavoring agents and sweetening agents. Preservatives and coloring agents may also be added.

The discrete units into which the suspension is formed may be liquid units, for example contained within the pockets of a suitable mold. Alternatively, the suspension may be in the form of solid units, for example frozen units. In certain aspects each unit will contain up to 100 mg of the composition, e.g., 1-100 mg, 1-50 mg, 1-25 mg, 1-20 mg, 1-15 mg, 1-10 mg, or 1-5 mg. Unit dosage forms of the composition in disintegrating form are encompassed by the example formulation process (also provided in FIG. 2). The desired quantities of the suspension may be filled into molds using an automatic filling instrument which delivers a predetermined dose into each of the depressions in each mold.

Removal of the continuous phase from the discrete units of the suspension comprising the active ingredient may be carried out by techniques well known to those skilled in the art. In a non-limiting example embodiment, when the discrete units are in a liquid form, they will generally be frozen prior to drying. The suspension contained within the pockets of a suitable mold is frozen, for example by passing a gaseous cooling medium such as liquid nitrogen over the mold or by inserting the mold into a nitrogen spray freezing chamber. Alternatively, the mold may be cooled by passing the mold over a cold surface. Once the dosage forms have been frozen, the mold may be stored in a cold store prior to drying.

Frozen discrete units may be dried by freeze drying according to techniques which are well known in the art. The continuous phase, for example water, is sublimed in a freeze-drying process under a reduced pressure which transforms the solid phase solvent (ice) directly into a vapor. In certain aspects, the freeze-drying process will be carried out in a freeze-drying chamber typically operating under a vacuum of 0.1 to 1.0 mBar for a period of time from 180 to 500 minutes. This process may be advantageously used to prepare oral solid disintegrating dosage forms of various formulations.

In some embodiments the oral dosage form disintegrates within 30 seconds, 20 sections, or less than 10 seconds, of being placed in the oral cavity. For instance, it disintegrates within 1 to 30, 5 to 30, 10 to 30, 1 to 20, 5 to 20, 5 to 20, 1 to 10, or 1 to 5 seconds.

### Non-Limiting Example Embodiments

1. A composition comprising a growth hormone-releasing hormone (GHRH) peptide and glycine.
2. The composition of embodiment 1, wherein the glycine facilitates absorption of the peptide.
3. The composition of embodiment 1 or embodiment 2, wherein about 50 μg to about 3000 μg, about 50 μg to about 500 μg, about 100 μg to about 500 μg, about 200 μg to about 500 μg, about 50 μg to about 400 μg, about 50 μg to about 300 μg, about 100 μg to about 300 μg, or about 200 to about 300 μg of glycine is present in the composition.
4. The composition of any one of embodiments 1-3, wherein about 300 μg to about 700 μg, about 800 μg to about 1200 μg, about 1300 μg to about 1700 μg, about 1800 μg to about 2200 μg, about 2300 μg to about 2700 μg, about 200 μg to about 300 μg, or about 225 μg to about 300 μg, about 250 μg to about 300 μg, about 200 to about 275 μg, about 200 to about 250 μg, or about 225 μg to about 275 μg of glycine is present in the composition.
5. The composition of any one of embodiments 1-4, wherein about 200 μg, about 210 μg, about 220 μg, about 230 μg, about 240 μg, about 250 μg, about 260 μg, about 270 μg, about 280 μg, about 290 μg, about 300 μg, about 450 ug, about 460 ug, about 470 ug, about 480 ug, about 490 ug, about 500 ug, about 510 ug, about 520 ug, about 530 ug, about 540 ug, about 550 ug, about 950 ug, about 960 ug, about 970 ug, about 980 ug, about 990 ug, about 1000 ug, about 1010 ug, about 1020 ug, about 1030 ug, about 1040 ug, about 1050 ug, about 1450 ug, about 1460 ug, about 1470 ug, about 1480 ug, about 1490 ug, about 1500 ug, about 1510 ug, about 1520 ug, about 1530 ug, about 1540 ug, about 1550 ug, about 1950 ug, about 1960 ug, about 1970 ug, about 1980 ug, about 1990 ug, about 2000 ug, about 2010 ug, about 2020 ug, about 2030 ug, about 2040 ug, about 2050 ug, about 2450 ug, about 2460 ug, about 2470 ug, about 2480 ug, about 2490 ug, about 2500 ug, about 2510 ug, about 2520 ug, about 2530 ug, about 2540 ug, or about 2550 ug of glycine is present in the composition.
6. The composition of any one of embodiments 1-5, wherein about 1000 ug of glycine is present in the composition.
7. The composition of any one of embodiments 1-5, wherein about 1500 ug of glycine is present in the composition.
8. The composition of any one of embodiments 1-5, wherein about 2000 ug of glycine is present in the composition.
9. The composition of any one of embodiments 1-5, wherein about 2500 ug of glycine is present in the composition.
10. The composition of any one of embodiments 1-5, wherein about 3000 ug of glycine is present in the composition.
11. The composition of any one of embodiments 1-10, comprising a delivery-enhancing agent.
12. A composition comprising a growth hormone-releasing hormone (GHRH) peptide and a delivery-enhancing agent.
13. The composition of clam 11 or embodiment 12, where the delivery-enhancing agent increases absorption of the peptide across mucous membranes.
14. The composition of any one of embodiments 11-13, wherein the delivery-enhancing agent comprises glycine, an aggregation inhibitory agent, a charge-modifying agent, a pH control agent, a degradative enzyme inhibitory agent, a mucolytic or mucus clearing agent, a ciliostatic agent, a modulatory agent of epithelial junction physiology, a vasodilator agent, a selective transport-enhancing agent, or a membrane penetration-enhancing agent, or a combination of two or more thereof.
15. The composition of embodiment 14, wherein the delivery-enhancing agent comprises the membrane penetration-enhancing agent.
16. The composition of any one of embodiments 11-15, wherein the delivery-enhancing agent comprises (i) a surfactant, (ii) a bile salt, (ii) a phospholipid additive, mixed micelle, liposome, or carrier, (iii) an alcohol, (iv) an enamine, (v) an NO donor compound, (vi) a long-chain amphipathic molecule, (vii) a small hydrophobic penetration enhancer, (viii) sodium or a salicylic acid derivative, (ix) a glycerol ester of acetoacetic acid, (x) a cyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, (x) an inhibitor of cholesterol synthesis, (xi) glycine, and (xii) any combination of the delivery-enhancing agents recited in (i)-(xi).
17. The composition of any one of embodiments 11-15, wherein the delivery-enhancing agent comprises an alkyl glycoside.
18. The composition of embodiment 17, wherein the alkyl glycoside comprises a saccharide joined to a hydrophobic alkyl.
19. The composition of embodiment 18, wherein the saccharide comprises glucose, maltose, maltotriose, maltotetrose, sucrose or trehalose, or a combination of two or more thereof.
20. The composition of embodiment 18 or embodiment 19, wherein the hydrophobic alkyl has a length of about 9 to about 24 carbons.
21. The composition of embodiment 20, wherein the hydrophobic alkyl has a length of about 10 to about 14 carbons.
22. The composition of any one of embodiments 17-21, wherein the alkyl glycoside comprises octyl-, nonyl-, decyl-, undecyl-, dodecyl, tridecyl, tctradecyl, pentadecyl, octadecyl a- or β-D-maltoside,-giucoside or sucroside.
23. The composition of any one of embodiments 17-22, wherein the alkyl glycoside comprises dodecyl maltoside, tetradecyl maltoside, sucrose dodecanoate, sucrose monostearate, sucrose distearate, or a combination of two or more thereof.
24. The composition of embodiment 23, wherein the alkyl glycoside comprises dodecyl maltoside.
25. The composition of embodiment 24, wherein the alkyl glycoside comprises n-dodecyl-β-D-maltoside.
26. The composition of any one of embodiments 11-25, wherein about 1 μg to about 30 μg, about 1 μg to about 20 μg, about 1 μg to about 15 μg, about 1 μg to about 10 μg, about 5 μg to about 30 μg, about 5 μg to about 20 μg, about 5 μg to about 15 μg, or about 5 to about 10 μg of the delivery-enhancing agent is present in the composition.
27. The composition of any one of embodiments 11-25, wherein about 8 μg to about 15 μg, about 8 μg to about 14 μg, about 8 μg to about 13 μg, about 8 μg to about 12 μg, about 9 μg to about 15 μg, about 9 μg to about 14 μg, about 9 μg to about 13 μg, about 9 μg to about 12 μg, about 10 μg to about 15 μg, about 10 μg to about 14 μg, about 10 μg to about 13 μg, about 10 μg to about 12 μg, or about 11 μg to about 13 μg of the delivery-enhancing agent is present in the composition.
28. The composition of any one of embodiments 11-25, wherein about 8 μg, about 9 μg, about 10 μg, about 11 μg, about 12 μg, about 13 μg, about 14 μg, or about 15 μg of the delivery-enhancing agent is present in the composition.
29. The composition of any one of embodiments 1-28, further comprising a stimulatory agent.
30. A composition comprising a growth hormone-releasing hormone (GHRH) peptide and a stimulatory agent.
31. The composition of embodiment 29 or embodiment 30, wherein the stimulatory agent induces secretion of human growth hormone.
32. The composition of any one of embodiments 29-31, wherein the stimulatory agent comprises an amino acid.
33. The composition of any one of embodiments 29-32, wherein the stimulatory agent comprises a neurotransmitter.
34. The composition of any one of embodiments 29-33, wherein the stimulatory agent comprises glycine, choline, arginine, ornithine, glycine, lysine, glutamine, or niacin (vitamin B3), or a combination of two or more thereof.
35. The composition of embodiment 34, wherein the stimulatory agent comprises glycine.
36. The composition of any one of embodiments 29-34, wherein about 50 μg to about 3000 μg, about 50 μg to about 500 μg, about 100 μg to about 500 μg, about 200 μg to about 500 μg, about 50 μg to about 400 μg, about 50 μg to about 300 μg, about 100 μg to about 300 μg, or about 200 to about 300 μg of the stimulatory agent is present in the composition.
37. The composition of any one of embodiments 29-34, wherein about 300 μg to about 700 μg, about 800 μg to about 1200 μg, about 1300 μg to about 1700 μg, about 1800 μg to about 2200 μg, about 2300 μg to about 2700 μg, about 200 μg to about 300 μg, or about 225 μg to about 300 μg, about 250 μg to about 300 μg, about 200 to about 275 μg, about 200 to about 250 μg, or about 225 μg to about 275 μg of the stimulatory agent is present in the composition.
38. The composition of any one of embodiments 29-34, wherein about 200 μg, about 210 μg, about 220 μg, about 230 μg, about 240 μg, about 250 μg, about 260 μg, about 270 μg, about 280 μg, about 290 μg, about 300 μg, about 450 ug, about 460 ug, about 470 ug, about 480 ug, about 490 ug, about 500 ug, about 510 ug, about 520 ug, about 530 ug, about 540 ug, about 550 ug, about 950 g, about 960 ug, about 970 ug, about 980 ug, about 990 ug, about 1000 ug, about 1010 ug, about 1020 ug, about 1030 ug, about 1040 ug, about 1050 ug, about 1450 ug, about 1460 ug, about 1470 ug, about 1480 ug, about 1490 ug, about 1500 ug, about 1510 ug, about 1520 ug, about 1530 ug, about 1540 ug, about 1550 ug, about 1950 ug, about 1960 ug, about 1970 ug, about 1980 ug, about 1990 ug, about 2000 ug, about 2010 ug, about 2020 ug, about 2030 ug, about 2040 ug, about 2050 ug, about 2450 ug, about 2460 ug, about 2470 ug, about 2480 ug, about 2490 ug, about 2500 ug, about 2510 ug, about 2520 ug, about 2530 ug, about 2540 ug, or about 2550 ug of the stimulatory agent is present in the composition.
39. The composition of any one of embodiments 29-34, wherein about 1000 ug of stimulatory agent is present in the composition.
40. The composition of any one of embodiments 29-34, wherein about 1500 ug of stimulatory agent is present in the composition.
41. The composition of any one of embodiments 29-34, wherein about 2000 ug of stimulatory agent is present in the composition.
42. The composition of any one of embodiments 29-34, wherein about 2500 ug of stimulatory agent is present in the composition.
43. The composition of any one of embodiments 29-34, wherein about 3000 ug of stimulatory agent is present in the composition.
44. The composition of any one of embodiments 1-43, further comprising a steroid.
45. A composition comprising a growth hormone-releasing hormone (GHRH) peptide and a steroid.
46. The composition of embodiment 44 or embodiment 45, wherein the steroid comprises a steroid glycoside.
47. The composition of embodiment 44 or embodiment 45, wherein the steroid comprises a triterpene saponin.
48. The composition of any one of embodiments 44-47, wherein the steroid comprises a ginsenoside.
49. The composition of embodiment 48, wherein the ginsenoside comprises ginsenoside Rb1, ginsenoside Rb2, ginsenoside Rb3, ginsenoside Rc, ginsenoside Rd, ginsenoside Re, ginsenoside Rg1, ginsenoside Rg2, ginsenoside Rg3, or ginsenoside Rh1, or a combination of two or more thereof.
50. The composition of embodiment 49, comprising ginsenoside Rg3.
51. The composition of any one of embodiments 44-50, wherein about 200 μg to about 2000 μg, about 500 μg to about 2000 μg, about 500 μg to about 1500 μg, about 500 μg to about 1000 μg, about 1000 μg to about 2000 μg, or about 1000 to about 1500 μg of the steroid is present in the composition.
52. The composition of any one of embodiments 44-50, wherein about 800 μg to about 1200 μg, or about 900 μg to about 1100 μg, or about 950 μg to about 1050 μg of the steroid is present in the composition.
53. The composition of any one of embodiments 44-50, wherein about 900 μg, about 950 μg, about 1000 μg, about 1050 μg, or about 1100 μg of the steroid is present in the composition.
54. The composition of any one of embodiments 1-53, further comprising a vitamin.
55. A composition comprising a growth hormone-releasing hormone (GHRH) peptide and a vitamin.
56. The composition of embodiment 54 or embodiment 55, wherein the vitamin increases NAD+ levels.
57. The composition of any one of embodiments 54-56, wherein the vitamin comprises vitamin A, vitamin C, vitamin D, vitamin E, vitamin K, vitamin B1, vitamin B2, vitamin B3, vitamin B5, vitamin B6, vitamin B7, vitamin B9, vitamin B12, or a combination of two or more thereof.
58. The composition of any one of embodiments 54-57, wherein the vitamin comprises vitamin B3 and/or vitamin B12.
59. The composition of embodiment 58, comprising vitamin B3.
60. The composition of embodiment 59, wherein the vitamin B3 comprises nicotinamide or niacin.
61. The composition of embodiment 60, wherein the nicotinamide comprises nicotinamide riboside.
62. The composition of any one of embodiments 58-61, comprising vitamin B12.
63. The composition of embodiment 62, wherein the vitamin B12 comprises methylcobalamin or cyanocobalamin.
64. The composition of embodiment 63, wherein the vitamin B12 comprises methylcobalamin.
65. The composition of any one of embodiments 54-64, wherein about 200 μg to about 2000 μg, about 500 μg to about 2000 μg, about 500 μg to about 1500 μg, about 500 μg to about 1000 μg, about 1000 μg to about 2000 μg, or about 1000 to about 1500 μg of the vitamin is present in the composition.
66. The composition of any one of embodiments 54-64, wherein about 800 μg to about 1200 μg, or about 900 μg to about 1100 μg, or about 950 μg to about 1050 μg of the vitamin is present in the composition.
67. The composition of any one of embodiments 54-64, wherein about 900 μg, about 950 μg, about 1000 μg, about 1050 μg, or about 1100 μg of the vitamin is present in the composition.
68. The composition of any one of embodiments 54-64, wherein about 400 μg to about 4000 μg, about 1000 μg to about 4000 μg, about 1000 μg to about 3000 μg, about 1000 μg to about 2000 μg, about 2000 μg to about 4000 μg, or about 2000 to about 3000 μg of the vitamin is present in the composition.
69. The composition of any one of embodiments 54-64, wherein about 1600 μg to about 2400 μg, or about 1800 μg to about 2200 μg, or about 1900 μg to about 1100 μg of the vitamin is present in the composition.
70. The composition of any one of embodiments 54-64, wherein about 1900 μg, about 1950 μg, about 2000 μg, about 2050 μg, or about 2100 μg of the vitamin is present in the composition.
71. The composition of any one of embodiments 54-70, wherein the vitamin comprises two or more vitamins.
72. The composition of embodiment 71, wherein the two or more vitamins comprise vitamin B3 and vitamin B12.
73. The composition of embodiment 71 or embodiment 72, wherein about 200 μg to about 2000 μg, about 500 μg to about 2000 μg, about 500 μg to about 1500 μg, about 500 μg to about 1000 μg, about 1000 μg to about 2000 μg, or about 1000 to about 1500 μg of each vitamin is present in the composition.
74. The composition of embodiment 71 or embodiment 72, wherein about 800 μg to about 1200 μg, or about 900 μg to about 1100 μg, or about 950 μg to about 1050 μg of each vitamin is present in the composition.
75. The composition of embodiment 71 or embodiment 72, wherein about 900 μg, about 950 μg, about 1000 μg, about 1050 μg, or about 1100 μg of each vitamin is present in the composition.
76. The composition of any one of embodiments 1-75, further comprising an excipient.
77. A composition comprising a growth hormone-releasing hormone (GHRH) peptide and an excipient.
78. The composition of embodiment 76 or embodiment 77, wherein the excipient is a penetration enhancer.
79. The composition of any one of embodiments 76-78, wherein the excipient comprises an alginate.
80. The composition of any one of embodiments 76-79, wherein the excipient comprises sodium alginate.
81. The composition of any one of embodiments 76-80, wherein about 1 μg to about 30 μg, about 1 μg to about 20 μg, about 1 μg to about 15 μg, about 1 μg to about 10 μg, about 5 μg to about 30 μg, about 5 μg to about 20 μg, about 5 μg to about 15 μg, or about 5 to about 10 μg of the excipient is present in the composition.
82. The composition of any one of embodiments 76-80, wherein about 8 μg to about 15 μg, about 8 μg to about 14 μg, about 8 μg to about 13 μg, about 8 μg to about 12 μg, about 9 μg to about 15 μg, about 9 μg to about 14 μg, about 9 μg to about 13 μg, about 9 μg to about 12 μg, about 10 μg to about 15 μg, about 10 μg to about 14 μg, about 10 μg to about 13 μg, about 10 μg to about 12 μg, or about 11 μg to about 13 μg of the excipient is present in the composition.
83. The composition of any one of embodiments 76-80, wherein about 8 μg, about 9 μg, about 10 μg, about 11 μg, about 12 μg, about 13 μg, about 14 μg, or about 15 μg of the excipient is present in the composition.
84. The composition of any one of embodiments 1-83, wherein the GHRH peptide comprises amino acids 1-29 of human GHRH.
85. The composition of any one of embodiments 1-84, wherein the GHRH peptide comprises a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1 (YADAIFTNSY RKVLGQLSAR KLLQDIMSR).
86. The composition of any one of embodiments 1-84, wherein the GHRH peptide comprises a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 (YADAIFTNSY RKVLGQLSAR KLLQDIMSR-NH2).
87. The composition of any one of embodiments 1-84, wherein the GHRH peptide comprises a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 1.
88. The composition of any one of embodiments 1-84, wherein the GHRH peptide comprises SEQ ID NO: 1 (YADAIFTNSY RKVLGQLSAR KLLQDIMSR).
89. The composition of any one of embodiments 1-84, wherein the GHRH peptide comprises SEQ ID NO: 2 (YADAIFTNSY RKVLGQLSAR KLLQDIMSR-NH2).
90. The composition of any one of embodiments 1-89, wherein about 200 μg to about 2000 μg, about 500 μg to about 2000 μg, about 500 μg to about 1500 μg, about 500 μg to about 1000 μg, about 1000 μg to about 2000 μg, or about 1000 to about 1500 μg of the GHRH peptide is present in the composition.
91. The composition of any one of embodiments 1-89, wherein about 800 μg to about 1200 μg, or about 900 μg to about 1100 μg, or about 950 μg to about 1050 μg of the GHRH peptide is present in the composition.
92. The composition of any one of embodiments 1-89, wherein about 900 μg, about 950 μg, about 1000 μg, about 1050 μg, or about 1100 μg of the GHRH peptide is present in the composition.
93. The composition of any one of embodiments 1-92, formulated in a tablet.
94. The composition of embodiment 83, formulated in an oral dissolving tablet.
95. The composition of any one of embodiments 1-94, wherein the composition disintegrates in water in less than about 30 seconds, 29 seconds, 28 seconds, 27 seconds, 26 seconds, 25 seconds, 24 seconds, 23 seconds, 22 seconds, 21 seconds, 20 seconds, 19 seconds, 18 seconds, 17 seconds, 16 seconds, 15 seconds, 14 seconds, 13 seconds, 12 seconds, 11 seconds, 10 seconds, 9 seconds, 8 seconds, 7 seconds, 6 seconds, or 5 seconds.
96. The composition of embodiment 95, wherein the composition disintegrates in water in less than about 10 seconds.
97. The composition of any one of embodiments 1-96, wherein the composition is prepared in a process comprising lyophilization.
98. The composition of any one of embodiment 1-97, wherein the composition is prepared in a unit dosage form having about 1 mg to about 10 mg weight of active ingredient.
99. The composition of embodiment 98, wherein the active ingredient comprises the GHRH peptide, and optionally one or more of the following: glycine, delivery-enhancing agent, stimulatory agent, steroid, vitamin, and excipient.
100. The composition of embodiment 98, wherein the active ingredient comprises the GHRH peptide and glycine, and optionally an excipient.
101. The composition of any one of embodiments 98-100, wherein the unit dosage form has about 1, 2, 3, 4, 5, 6, or 7 mg of active ingredient.
102. A method of preparing a tablet comprising the composition of any one of embodiments 1-101, the method comprising lyophilizing a formulation comprising the composition of any one of embodiments 1-101.
103. The method of embodiment 102, wherein the formulation comprises a continuous phase.
104. The method of embodiment 103, wherein the continuous phase comprises water.
105. A method of improving the health of a subject, the method comprising administering to the subject a composition of any one of embodiments 1-104.
106. The method of embodiment 105, wherein the administration is oral.
107. The method of embodiment 105 or embodiment 106, wherein the administration is sublingual or buccal.
108. The method of any one of embodiments 105-107, wherein the composition increases testosterone, improves endurance, improves performance, boosts the immune system, boosts metabolism, naturally modulates hGH, improves libido, improves cognitive function, improves mood, increases energy, improves skin texture & tone, repairs DNA and maintains cell health, increases strength, helps to build lean muscles, improves muscle mass, improves skin health, improves bone health, has a calming effect, has a deep sleep effect, reduces time it takes to fall asleep, or boosts daytime alertness, or any combination of two or more thereof, as compared to the subject prior to administration of the composition.
109. The method of any one of embodiments 105-108, wherein the composition is administered in a regimen comprises two or more doses.
110. The method of embodiment 109, wherein the two or more doses is 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 doses.
111. The method of embodiment 109, wherein the two or more doses is 2, 3, 4, 5, 6, 7, 8, 9, or 10 doses.
112. The method of embodiment 109, wherein the two or more doses is 5 doses.
113. The method of any one of embodiments 109-112, wherein each dose is administered daily.
114. The method of any one of embodiments 109-112, wherein each dose is administered every other day, every two days, every three days, every four days, every five days, every six days, every seven days, once a week, once every two weeks, once every three weeks, or once every four weeks.
115. The method of any one of embodiments 109-112, wherein the regimen comprises the two or more doses, followed by one or more days where the composition is not administered to the subject.
116. The method of embodiment 115, wherein the one or more days is 1, 2, 3, 4, 5, 6, or 7 days.
117. The method of embodiment 115, wherein the one or more days is 2 days.
118. The method of any one of embodiments 109-117, wherein the regimen comprises five daily doses, followed by two days where the composition is not administered to the subject.
119. The method of any one of embodiments 109-118, wherein the regimen is repeated one or more times.
120. The method of any one of embodiments 109-119, wherein the regimen is repeated weekly, every two weeks, every three weeks, or every four weeks.
121. The method of any one of embodiments 109-120, wherein the regimen is repeated weekly.

### Further Non-Limiting Example Embodiments

1. A composition comprising a growth hormone-releasing hormone (GHRH) peptide and glycine.
2. The composition of embodiment 1, wherein about 50 μg to about 3000 μg of glycine is present in the composition.
3. The composition of embodiment 1 or embodiment 2, comprising a delivery-enhancing agent.
4. A composition comprising a growth hormone-releasing hormone (GHRH) peptide and a delivery-enhancing agent.
5. The composition of embodiment 3 or embodiment 4, wherein the delivery-enhancing agent comprises an alkyl glycoside.
6. The composition of embodiment 5, wherein the alkyl glycoside comprises n-dodecyl-β-D-maltoside.
7. The composition of any one of embodiments 3-6, wherein the delivery-enhancing agent comprises a membrane penetration-enhancing agent.
8. The composition of any one of embodiments 3-7, wherein the delivery-enhancing agent comprises (i) a surfactant, (ii) a bile salt, (ii) a phospholipid additive, mixed micelle, liposome, or carrier, (iii) an alcohol, (iv) an enamine, (v) an NO donor compound, (vi) a long-chain amphipathic molecule, (vii) a small hydrophobic penetration enhancer, (viii) sodium or a salicylic acid derivative, (ix) a glycerol ester of acetoacetic acid, (x) a cyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, (x) an inhibitor of cholesterol synthesis, (xi) glycine, and (xii) any combination of the delivery-enhancing agents recited in (i)-(xi).
9. The composition of any one of embodiments 3-8, wherein about 1 μg to about 30 μg of the delivery-enhancing agent is present in the composition.
10. The composition of any one of embodiments 1-9, further comprising a stimulatory agent.
11. A composition comprising a growth hormone-releasing hormone (GHRH) peptide and a stimulatory agent.
12. The composition of embodiment 10 or embodiment 11, wherein the stimulatory agent induces secretion of human growth hormone.
13. The composition of any one of embodiments 10-12, wherein about 50 μg to about 3000 μg of the stimulatory agent is present in the composition.
14. The composition of any one of embodiments 1-13, further comprising a steroid.
15. A composition comprising a growth hormone-releasing hormone (GHRH) peptide and a steroid.
16. The composition of embodiment 14 or embodiment 15, wherein the steroid comprises ginsenoside Rg3.
17. The composition of any one of embodiments 14-16, wherein about 200 μg to about 2000 μg of the steroid is present in the composition.
18. The composition of any one of embodiments 1-17, further comprising a vitamin.
19. A composition comprising a growth hormone-releasing hormone (GHRH) peptide and a vitamin.
20. The composition of embodiment 18 or embodiment 19, wherein the vitamin comprises nicotinamide riboside.
21. The composition of any one of embodiments 18-20, wherein the vitamin comprises methylcobalamin.
22. The composition of any one of embodiments 18-20, wherein about 200 μg to about 2000 μg of the vitamin is present in the composition.
23. The composition of any one of embodiments 1-22, further comprising an excipient.
24. A composition comprising a growth hormone-releasing hormone (GHRH) peptide and an excipient.
25. The composition of embodiment 23 or embodiment 24, wherein the excipient is a penetration enhancer.
26. The composition of any one of embodiments 23-25, wherein the excipient comprises sodium alginate.
27. The composition of any one of embodiments 23-26, wherein about 1 μg to about 30 μg of the excipient is present in the composition.
28. The composition of any one of embodiments 1-27, wherein the GHRH peptide comprises amino acids 1-29 of human GHRH.
29. The composition of any one of embodiments 1-28, wherein the GHRH peptide comprises a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1 (YADAIFTNSY RKVLGQLSAR KLLQDIMSR) or SEQ ID NO: 2 (YADAIFTNSY RKVLGQLSAR KLLQDIMSR-NH2).
30. The composition of any one of embodiments 1-29, wherein about 200 μg to about 2000 μg of the GHRH peptide is present in the composition.
31. The composition of any one of embodiments 1-30, formulated in an oral dissolving tablet.
32. The composition of embodiment 31, wherein the composition disintegrates in water in less than about 30 seconds.
33. The composition of any one of embodiments 1-32, wherein the composition is prepared in a process comprising lyophilization.
34. A method of preparing a tablet comprising the composition of any one of embodiments 1-33, the method comprising lyophilizing a formulation comprising the composition of any one of embodiments 1-33.
35. A method of improving the health of a subject, the method comprising administering to the subject a composition of any one of embodiments 1-34.
36. The method of embodiment 35, wherein the composition increases testosterone, improves endurance, improves performance, boosts the immune system, boosts metabolism, naturally modulates hGH, improves libido, improves cognitive function, improves mood, increases energy, improves skin texture & tone, repairs DNA and maintains cell health, increases strength, helps to build lean muscles, improves muscle mass, improves skin health, improves bone health, has a calming effect, has a deep sleep effect, reduces time it takes to fall asleep, or boosts daytime alertness, or any combination of two or more thereof, as compared to the subject prior to administration of the composition.

### Therapeutic Effect/Improved Health

Embodiments of peptide compositions and formulations comprising compositions herein are adapted to have therapeutic effects when taken by a subject. A therapeutic effect includes an improvement in the health of the subject. In certain aspects, such effects include increased testosterone, improved endurance, improved performance, and a boosted metabolism. Other effects include increased strength, improved muscle mass, improved skin and bone health, and improved ability to build lean muscle. In certain aspects, peptide compositions naturally modulate hGH, improve libido, and improve cognitive function and mood. Certain embodiments of the composition repair DNA and maintain cell health, and boost the immune system. Furthermore, certain embodiments of the composition have deep sleep and calming effects, reduce the time it takes to fall asleep and boost daytime alertness, increase energy, and improve skin texture and tone. Embodiments of the composition may result in a combination of two or more of these effects, as compared to the subject prior to taking the composition.

In some embodiments, a subject refers to any animal, including, but not limited to, humans, non-human primates, rodents, and domestic and game animals, which is to be the recipient of a particular treatment. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat; canine species, e.g., dog, fox, wolf; avian species, e.g., chicken, emu, ostrich; and fish, e.g., trout, catfish and salmon. In various embodiments, a subject can be one who has been previously diagnosed with or identified as suffering from or having a condition in need of treatment. In certain embodiments, the subject is a human. In various embodiments, the subject previously diagnosed with or identified as suffering from or having a condition may or may not have undergone treatment for a condition. In some embodiments, a subject can also be one who has not been previously diagnosed as having a condition (e.g., a subject who exhibits one or more risk factors for a condition). In some aspects a subject in need of treatment for a particular condition can be a subject having that condition, diagnosed as having that condition, or at risk of developing that condition. In some embodiments, the subject is a patient that has been diagnosed with a condition described herein. In some instances, the subject is suffering from a symptom related to a condition disclosed herein.

In some embodiments, a therapeutically effective amount refers to an amount of a peptide and/or composition effective to treat a condition in a subject or mammal. In some cases, a therapeutically effective amount reduces the severity of symptoms of the condition.

Non-limiting conditions for treatment with a peptide and/or composition herein include: low testosterone, low endurance, low performance, weak immune system, low metabolism, low libido, low cognitive function, poor mood, low energy, weak skin texture, weak skin ton, DNA damage, cellular damage, weakness, low musculature, low muscle mass, poor skin health, poor bone health, anxiety, insomnia and other sleep disorders, difficulty falling asleep, and poor daytime alertness. In some cases, low or poor refers to a level that is below an average level of healthy subjects. In some cases, low or poor refers to a level that is below the level of a healthy subject. The healthy subject(s) may be of similar age as the subject with the condition.

The compositions may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of therapeutic compound (including activity, pharmacokinetics, pharmacodynamics, and bioavailability), the physiological condition of the subject (including age, sex, disease type and stage, general physical condition, responsiveness to a given dosage), the nature of the carrier or carriers in the formulation, and the route of administration. The therapeutically effective amount may be determined by monitoring a subject's response to administration of a composition and adjusting the dosage accordingly.

The duration of treatment depends upon the subject's clinical progress and responsiveness to therapy.

Provided below are descriptions of embodiments of dosing regimens for compositions. In one aspect, a composition is orally administered to a subject to improve the health of the subject. In some embodiments oral administration is sublingual. In some embodiments oral administration is buccal. In some aspects, buccal administration is done such that a dissolving/dispersing (e.g., fast-dissolve or fast-dispersing) tablets of the peptide composition are placed between the cheek and gum or into close association with buccal tissue inside the mouth. In some aspects, buccal administration results in large proportions of the peptide composition directly absorbed into systemic circulation resulting in a smaller amount subsequently undergoing first pass elimination in the liver. In a nonlimiting embodiment, high oral bioavailability is realized upon administering the peptide compound inside the central portion of the upper lip, between the inside of the lip and gums, directly below the nose. For compositions comprising an alkyl saccharide, the alkyl saccharide content can be reduced to attenuate buccal absorption so that a portion of the peptide is immediately absorbed buccally for rapid onset, but the rest is absorbed through the slower gastric absorption process.

In some embodiments the composition is administered in a regimen comprising two or more doses, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 doses. In one embodiment the two or more dose regimen is 2, 3, 4, 5, 6, 7, 8, 9, or 10 doses. In some embodiments the two or more doses is 5 doses. Administration of the composition varies in some embodiments; a dose of the composition may be administered every day, every other day, every two days, every three days, every four days, every five days, every six days, every seven days, once a week, once every two weeks, once every three weeks, or once every four weeks. Furthermore, in some instances, the dosing regimen may change, e.g., a regimen of two or more doses (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 doses) for a number of days may be followed by one or more days where the composition is not administered to the subject. In some embodiments, the one or more days is 1, 2, 3, 4, 5, 6, or 7 days. So, as a nonlimiting example, a subject may take a dose every day for a period of two weeks followed by a period of 7 wherein the subject is not administered any doses. In an additional nonlimiting embodiment, the subject may follow a dosing regimen wherein the subject is administered 5 daily doses followed by two days where the composition is not administered. In some embodiments, a given regimen may be repeated one or more times, e.g. weekly, every two weeks, every three weeks, or every four weeks.

In some embodiments, the method comprises administering to the subject one or more doses of the composition. In some embodiments, each dose comprises about 0.1 mg to about 8 mg of the composition, about 0.5 mg to about 7 mg, about 2 mg to about 6 mg, or about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 2.1 mg, about 2.2 mg, about 2.3 mg, about 2.4 mg, about 2.5 mg, about 2.6 mg, about 2.7 mg, about 2.8 mg, about 2.9 mg, about 3 mg, about 3.1 mg, about 3.2 mg, about 3.3 mg, about 3.4 mg, about 3.5 mg, about 3.6 mg, about 3.7 mg, about 3.8 mg, about 3.9 mg, about 4 mg, about 4.1 mg, about 4.2 mg, about 4.3 mg, about 4.4 mg, about 4.5 mg, about 4.6 mg, about 4.7 mg, about 4.8 mg, about 4.9 mg, about 5 mg, about 5.1 mg, about 5.2 mg, about 5.3 mg, about 5.4 mg, about 5.5 mg, about 5.6 mg, about 5.7 mg, about 5.8 mg, about 5.9 mg, about 6 mg, about 6.1 mg, about 6.2 mg, about 6.3 mg, about 6.4 mg, about 6.5 mg, about 6.6 mg, about 6.7 mg, about 6.8 mg, about 6.9 mg, about 7 mg, about 7.1 mg, about 7.2 mg, about 7.3 mg, about 7.4 mg, about 7.5 mg, about 7.6 mg, about 7.7 mg, about 7.8 mg, about 7.9 mg, or about 8 mg. In some embodiments, each dose comprises about 200 μg to about 2000 μg of GHRH peptide, e.g., about 200 μg to about 1800 μg, about 200 μg to about 1600 μg, about 200 μg to about 1400 μg, about 200 μg to about 1200 μg, about 200 μg to about 1000 μg, about 200 μg to about 800 μg, about 400 μg to about 2000 μg, about 400 μg to about 1800 μg, about 400 μg to about 1600 μg, about 400 μg to about 1400 μg, about 400 μg to about 1200 μg, about 400 μg to about 1000 μg, about 400 μg to about 800 μg, about 600 μg to about 2000 μg, about 600 μg to about 1800 μg, about 600 μg to about 1600 μg, about 600 μg to about 1400 μg, about 600 μg to about 1200 μg, about 600 μg to about 1000 μg, about 600 μg to about 800 μg, about 800 μg to about 2000 μg, about 800 μg to about 1800 μg, about 800 μg to about 1600 μg, about 800 μg to about 1400 μg, about 800 μg to about 1200 μg, about 800 μg to about 1000 μg, about 800 μg, about 850 μg, about 900 μg, about 950 μg, about 1000 μg, about 1050 μg, about 1100 μg, about 1150 μg, about 1200 μg, about 1250 μg, about 1300 μg, about 1400 μg, about 1500 μg, about 1600 μg, about 1800 μg, or about 2000 μg of GHRH peptide (e.g., a peptide comprising a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1 (YADAIFTNSY RKVLGQLSAR KLLQDIMSR), SEQ ID NO: 2 (YADAIFTNSY RKVLGQLSAR KLLQDIMSR-NH2), or a sequence of Table 1). In some embodiments, each dose comprises about 50 μg to about 3000 μg or about 50 μg to about 500 μg of stimulatory agent and/or neurotransmitter and/or amino acid (e.g., glycine), e.g., about 50 μg to about 3000 μg, about 50 μg to about 2900 μg, about 50 μg to about 2800 μg, about 50 μg to about 2700 μg, about 50 μg to about 2600 μg, about 50 μg to about 2500 μg, about 50 μg to about 2400 μg, about 50 μg to about 2300 μg, about 50 μg to about 2200 μg, about 50 μg to about 2100 μg, about 50 μg to about 2000 μg, about 50 μg to about 1900 μg, about 50 μg to about 1800 μg, about 50 μg to about 1700 μg, about 50 μg to about 1600 μg, about 50 μg to about 1500 μg, about 50 μg to about 1400 μg, about 50 μg to about 1300 μg, about 50 μg to about 1200 μg, about 50 μg to about 1100 μg, about 50 μg to about 1000 μg, about 50 μg to about 900 μg, about 50 μg to about 800 μg, about 50 μg to about 700 μg, about 50 μg to about 600 μg, about 100 μg to about 3000 μg, about 200 μg to about 3000 μg, about 300 μg to about 3000 μg, about 400 μg to about 3000 μg, about 500 μg to about 3000 μg, about 600 μg to about 3000 μg, about 700 μg to about 3000 μg, about 800 μg to about 3000 μg, about 900 μg to about 3000 μg, about 1000 μg to about 3000 μg, about 1100 μg to about 3000 μg, about 1200 μg to about 3000 μg, about 1300 μg to about 3000 μg, about 1400 μg to about 3000 μg, about 1500 μg to about 3000 μg, about 1600 μg to about 3000 μg, about 1700 μg to about 3000 μg, about 1800 μg to about 3000 μg, about 1900 μg to about 3000 μg, about 2000 μg to about 3000 μg, about 2100 μg to about 3000 μg, about 2200 μg to about 3000 μg, about 2300 μg to about 3000 μg, about 2400 μg to about 3000 μg, about 2500 μg to about 3000 μg, about 2600 μg to about 3000 μg, about 2700 μg to about 3000 μg, about 2800 μg to about 3000 μg, about 2900 μg to about 3000 μg, about 100 μg to about 2500 μg, about 200 μg to about 2500 μg, about 300 μg to about 2500 μg, about 400 μg to about 2500 μg, about 500 μg to about 2500 μg, about 600 μg to about 2500 μg, about 700 μg to about 2500 μg, about 800 μg to about 2500 μg, about 900 μg to about 2500 μg, about 1000 μg to about 2500 μg, about 1100 μg to about 2500 μg, about 1200 μg to about 2500 μg, about 1300 μg to about 2500 μg, about 1400 μg to about 2500 μg, about 1500 μg to about 2500 μg, about 1600 μg to about 2500 μg, about 1700 μg to about 2500 μg, about 1800 μg to about 2500 μg, about 1900 μg to about 2500 μg, about 2000 μg to about 2500 μg, about 2100 μg to about 2500 μg, about 2200 μg to about 2500 μg, about 2300 μg to about 2500 μg, about 2400 μg to about 2500 μg, about 100 μg to about 2000 μg, about 200 μg to about 2000 μg, about 300 μg to about 2000 μg, about 400 μg to about 2000 μg, about 500 μg to about 2000 μg, about 600 μg to about 2000 μg, about 700 μg to about 2000 μg, about 800 μg to about 2000 μg, about 900 μg to about 2000 μg, about 1000 μg to about 2000 μg, about 1100 μg to about 2000 μg, about 1200 μg to about 2000 μg, about 1300 μg to about 2000 μg, about 1400 μg to about 2000 μg, about 1500 μg to about 2000 μg, about 1600 μg to about 2000 μg, about 1700 μg to about 2000 μg, about 1800 μg to about 2000 μg, about 1900 μg to about 2000 μg, about 100 μg to about 1500 μg, about 200 μg to about 1500 μg, about 300 μg to about 1500 μg, about 400 μg to about 1500 μg, about 500 μg to about 1500 μg, about 600 μg to about 1500 μg, about 700 μg to about 1500 μg, about 800 μg to about 1500 μg, about 900 μg to about 1500 μg, about 1000 μg to about 1500 μg, about 1100 μg to about 1500 μg, about 1200 μg to about 1500 μg, about 1300 μg to about 1500 μg, about 1400 μg to about 1500 μg, about 100 μg to about 1000 μg, about 200 μg to about 1000 μg, about 300 μg to about 1000 μg, about 400 μg to about 1000 μg, about 500 μg to about 1000 μg, about 600 μg to about 1000 μg, about 700 μg to about 1000 μg, about 800 μg to about 1000 μg, about 900 μg to about 1000 μg, about 50 μg to about 500 μg, about 50 μg to about 450 μg, about 50 μg to about 400 μg, about 50 μg to about 350 μg, about 50 μg to about 300 μg, about 50 μg to about 250 μg, about 50 μg to about 200 μg, about 50 μg to about 150 μg, about 50 μg to about 100 μg, about 100 μg to about 500 μg, about 100 μg to about 450 μg, about 100 μg to about 400 μg, about 100 μg to about 350 μg, about 100 μg to about 300 μg, about 100 μg to about 250 μg, about 100 μg to about 200 μg, about 100 μg to about 150 μg, about 150 μg to about 500 μg, about 150 μg to about 450 μg, about 150 μg to about 400 μg, about 150 μg to about 350 μg, about 150 μg to about 300 μg, about 150 μg to about 250 μg, about 150 μg to about 200 μg, about 200 μg to about 500 μg, about 200 μg to about 450 μg, about 200 μg to about 400 μg, about 200 μg to about 350 μg, about 200 μg to about 300 μg, about 200 μg to about 250 μg, about 250 μg to about 500 μg, about 250 μg to about 450 μg, about 250 μg to about 400 μg, about 250 μg to about 350 μg, about 250 μg to about 300 μg, about 300 μg to about 700 μg, or about 350 μg to about 700 μg, about 400 μg to about 700 μg, about 450 μg to about 700 μg, about 500 μg to about 700 μg, about 300 μg to about 650 μg, about 350 μg to about 650 μg, about 400 μg to about 650 μg, about 450 μg to about 650 μg, about 500 μg to about 650 μg, about 300 μg to about 600 μg, about 350 μg to about 600 μg, about 400 μg to about 600 μg, about 450 μg to about 600 μg, about 500 μg to about 600 μg, about 300 μg to about 550 μg, about 350 μg to about 550 μg, about 400 μg to about 550 μg, about 450 μg to about 550 μg, about 500 μg to about 550 μg, about 300 μg to about 500 μg, about 350 μg to about 500 μg, about 400 μg to about 500 μg, or about 450 μg to about 500 μg of glycine is present in the composition. In some embodiments, about 800 μg to about 1200 μg, or about 850 μg to about 1200 μg, about 900 μg to about 1200 μg, about 950 μg to about 1200 μg, about 1000 μg to about 1200 μg, about 800 μg to about 1150 μg, about 850 μg to about 1150 μg, about 900 μg to about 1150 μg, about 950 μg to about 1150 μg, about 1000 μg to about 1150 μg, about 800 μg to about 1100 μg, about 850 μg to about 1100 μg, about 900 μg to about 1100 μg, about 950 μg to about 1100 μg, about 1000 μg to about 1100 μg, about 800 μg to about 1050 μg, about 850 μg to about 1050 μg, about 900 μg to about 1050 μg, about 950 μg to about 1050 μg, about 1000 μg to about 1050 μg, about 800 μg to about 1000 μg, about 850 μg to about 1000 μg, about 900 μg to about 1000 μg, or about 950 μg to about 1000 μg of glycine is present in the composition. In some embodiments, about 1300 μg to about 1700 μg, or about 1350 μg to about 1700 μg, about 1400 μg to about 1700 μg, about 1450 μg to about 1700 μg, about 1500 μg to about 1700 μg, about 1300 μg to about 1650 μg, about 1350 μg to about 1650 μg, about 1400 μg to about 1650 μg, about 1450 μg to about 1650 μg, about 1500 μg to about 1650 μg, about 1300 μg to about 1600 μg, about 1350 μg to about 1600 μg, about 1400 μg to about 1600 μg, about 1450 μg to about 1600 μg, about 1500 μg to about 1600 μg, about 1300 μg to about 1550 μg, about 1350 μg to about 1550 μg, about 1400 μg to about 1550 μg, about 1450 μg to about 1550 μg, about 1500 μg to about 1550 μg, about 1300 μg to about 1500 μg, about 1350 μg to about 1500 μg, about 1400 μg to about 1500 μg, or about 1450 μg to about 1500 μg of glycine is present in the composition. In some embodiments, about 1800 μg to about 2200 μg, or about 1850 μg to about 2200 μg, about 1900 μg to about 2200 μg, about 1950 μg to about 2200 μg, about 2000 μg to about 2200 μg, about 1800 μg to about 2150 μg, about 1850 μg to about 2150 μg, about 1900 μg to about 2150 μg, about 1950 μg to about 2150 μg, about 2000 μg to about 2150 μg, about 1800 μg to about 2100 μg, about 1850 μg to about 2100 μg, about 1900 μg to about 2100 μg, about 1950 μg to about 2100 μg, about 2000 μg to about 2100 μg, about 1800 μg to about 2050 μg, about 1850 μg to about 2050 μg, about 1900 μg to about 2050 μg, about 1950 μg to about 2050 μg, about 2000 μg to about 2050 μg, about 1800 μg to about 2000 μg, about 1850 μg to about 2000 μg, about 1900 μg to about 2000 μg, or about 1950 μg to about 2000 μg of glycine is present in the composition. In some embodiments, about 2300 μg to about 2700 μg, or about 2350 μg to about 2700 μg, about 2400 μg to about 2700 μg, about 2450 μg to about 2700 μg, about 2500 μg to about 2700 μg, 2300 μg to about 2650 μg, about 2350 μg to about 2650 μg, about 2400 μg to about 2650 μg, about 2450 μg to about 2650 μg, about 2500 μg to about 2650 μg, about 2300 μg to about 2600 μg, about 2350 μg to about 2600 μg, about 2400 μg to about 2600 μg, about 2450 μg to about 2600 μg, about 2500 μg to about 2600 μg, about 2300 μg to about 2550 μg, about 2350 μg to about 2550 μg, about 2400 μg to about 2550 μg, about 2450 μg to about 2550 μg, about 2500 μg to about 2550 μg, 2300 μg to about 2500 μg, about 2350 μg to about 2500 μg, about 2400 μg to about 2500 μg, or about 2450 μg to about 2500 μg, about 400 ug, about 410 ug, about 420 ug, about 430 ug, about 440 ug, about 450 ug, about 460 ug, about 470 ug, about 480 ug, about 490 ug, about 500 ug, about 510 ug, about 520 ug, about 530 ug, about 540 ug, about 550 ug, about 560 ug, about 570 ug, about 580 ug, about 590 ug, about 600 ug, about 900 ug, about 910 ug, about 920 ug, about 930 ug, about 940 ug, about 950 ug, about 960 ug, about 970 ug, about 980 ug, about 990 ug, about 1000 ug, about 1010 ug, about 1020 ug, about 1030 ug, about 1040 ug, about 1050 ug, about 1060 ug, about 1070 ug, about 1080 ug, about 1090 ug, about 1100 ug, about 1400 ug, about 1410 ug, about 1420 ug, about 1430 ug, about 1440 ug, about 1450 ug, about 1460 ug, about 1470 ug, about 1480 ug, about 1490 ug, about 1500 ug, about 1510 ug, about 1520 ug, about 1530 ug, about 1540 ug, about 1550 ug, about 1560 ug, about 1570 ug, about 1580 ug, about 1590 ug, about 1600 ug, about 1900 ug, about 1910 ug, about 1920 ug, about 1930 ug, about 1940 ug, about 1950 ug, about 1960 ug, about 1970 ug, about 1980 ug, about 1990 ug, about 2000 ug, about 2010 ug, about 2020 ug, about 2030 ug, about 2040 ug, about 2050 ug, about 2060 ug, about 2070 ug, about 2080 ug, about 2090 ug, about 2100 ug, about 2400 ug, about 2410 ug, about 2420 ug, about 2430 ug, about 2440 ug, about 2450 ug, about 2460 ug, about 2470 ug, about 2480 ug, about 2490 ug, about 2500 ug, about 2510 ug, about 2520 ug, about 2530 ug, about 2540 ug, about 2550 ug, about 2560 ug, about 2570 ug, about 2580 ug, about 2590 ug, about 2600 ug, about 50 μg, about 100 μg, about 150 μg, about 200 μg, about 250 μg, about 300 μg, about 350 μg, about 400 μg, about 450 μg, or about 500 μg of stimulatory agent and/or neurotransmitter and/or amino acid (e.g., glycine). In some embodiments, each dose comprises about 200 μg to about 2000 μg steroid (e.g., ginsenoside such as ginsenoside Rg3), e.g., about 200 μg to about 1800 μg, about 200 μg to about 1600 μg, about 200 μg to about 1400 μg, about 200 μg to about 1200 μg, about 200 μg to about 1000 μg, about 200 μg to about 800 μg, about 400 μg to about 2000 μg, about 400 μg to about 1800 μg, about 400 μg to about 1600 μg, about 400 μg to about 1400 μg, about 400 μg to about 1200 μg, about 400 μg to about 1000 μg, about 400 μg to about 800 μg, about 600 μg to about 2000 μg, about 600 μg to about 1800 μg, about 600 μg to about 1600 μg, about 600 μg to about 1400 μg, about 600 μg to about 1200 μg, about 600 μg to about 1000 μg, about 600 μg to about 800 μg, about 800 μg to about 2000 μg, about 800 μg to about 1800 μg, about 800 μg to about 1600 μg, about 800 μg to about 1400 μg, about 800 μg to about 1200 μg, about 800 μg to about 1000 μg, about 800 μg, about 850 μg, about 900 μg, about 950 μg, about 1000 μg, about 1050 μg, about 1100 μg, about 1150 μg, about 1200 μg, about 1250 μg, about 1300 μg, about 1400 μg, about 1500 μg, about 1600 μg, about 1800 μg, or about 2000 μg of steroid (e.g., ginsenoside such as ginsenoside Rg3). In some embodiments, each dose comprises about 200 μg to about 2000 μg vitamin (e.g., nicotinamide riboside and/or methylcobalamin), e.g., about 200 μg to about 1800 μg, about 200 μg to about 1600 μg, about 200 μg to about 1400 μg, about 200 μg to about 1200 μg, about 200 μg to about 1000 μg, about 200 μg to about 800 μg, about 400 μg to about 2000 μg, about 400 μg to about 1800 μg, about 400 μg to about 1600 μg, about 400 μg to about 1400 μg, about 400 μg to about 1200 μg, about 400 μg to about 1000 μg, about 400 μg to about 800 μg, about 600 μg to about 2000 μg, about 600 μg to about 1800 μg, about 600 μg to about 1600 μg, about 600 μg to about 1400 μg, about 600 μg to about 1200 μg, about 600 μg to about 1000 μg, about 600 μg to about 800 μg, about 800 μg to about 2000 μg, about 800 μg to about 1800 μg, about 800 μg to about 1600 μg, about 800 μg to about 1400 μg, about 800 μg to about 1200 μg, about 800 μg to about 1000 μg, about 800 μg, about 850 μg, about 900 μg, about 950 μg, about 1000 μg, about 1050 μg, about 1100 μg, about 1150 μg, about 1200 μg, about 1250 μg, about 1300 μg, about 1400 μg, about 1500 μg, about 1600 μg, about 1800 μg, or about 2000 μg of vitamin (e.g., nicotinamide riboside and/or methylcobalamin). In some cases, each dose comprises two vitamins, e.g., nicotinamide riboside and methylcobalamin, wherein each vitamin is present in the dose at about 200 μg to about 2000 μg, about 200 μg to about 1800 μg, about 200 μg to about 1600 μg, about 200 μg to about 1400 μg, about 200 μg to about 1200 μg, about 200 μg to about 1000 μg, about 200 μg to about 800 μg, about 400 μg to about 2000 μg, about 400 μg to about 1800 μg, about 400 μg to about 1600 μg, about 400 μg to about 1400 μg, about 400 μg to about 1200 μg, about 400 μg to about 1000 μg, about 400 μg to about 800 μg, about 600 μg to about 2000 μg, about 600 μg to about 1800 μg, about 600 μg to about 1600 μg, about 600 μg to about 1400 μg, about 600 μg to about 1200 μg, about 600 μg to about 1000 μg, about 600 μg to about 800 μg, about 800 μg to about 2000 μg, about 800 μg to about 1800 μg, about 800 μg to about 1600 μg, about 800 μg to about 1400 μg, about 800 μg to about 1200 μg, about 800 μg to about 1000 μg, about 800 μg, about 850 μg, about 900 μg, about 950 μg, about 1000 μg, about 1050 μg, about 1100 μg, about 1150 μg, about 1200 μg, about 1250 μg, about 1300 μg, about 1400 μg, about 1500 μg, about 1600 μg, about 1800 μg, or about 2000 μg. In some embodiments, each dose comprises about 1 μg to about 30 μg of excipient (e.g., alginate, such as sodium alginate), e.g., about 1 μg to about 30 μg, about 1 μg to about 29 μg, about 1 μg to about 28 μg, about 1 μg to about 27 μg, about 1 μg to about 26 μg, about 1 μg to about 25 μg, about 1 μg to about 24 μg, about 1 μg to about 23 μg, about 1 μg to about 22 μg, about 1 μg to about 21 μg, about 1 μg to about 20 μg, about 1 μg to about 19 μg, about 1 μg to about 18 μg, about 1 μg to about 17 μg, about 1 μg to about 16 μg, about 1 μg to about 15 μg, about 1 μg to about 14 μg, about 1 μg to about 13 μg, about 1 μg to about 12 μg, about 1 μg to about 11 μg, about 1 μg to about 10 μg, about 1 μg to about 9 μg, about 1 μg to about 8 μg, about 1 μg to about 7 μg, about 1 μg to about 6 μg, about 1 μg to about 5 μg, about 2 μg to about 30 μg, about 2 μg to about 29 μg, about 2 μg to about 28 μg, about 2 μg to about 27 μg, about 2 μg to about 26 μg, about 2 μg to about 25 μg, about 2 μg to about 24 μg, about 2 μg to about 23 μg, about 2 μg to about 22 μg, about 2 μg to about 21 μg, about 2 μg to about 20 μg, about 2 μg to about 19 μg, about 2 μg to about 18 μg, about 2 μg to about 17 μg, about 2 μg to about 16 μg, about 2 μg to about 15 μg, about 2 μg to about 14 μg, about 2 μg to about 13 μg, about 2 μg to about 12 μg, about 2 μg to about 11 μg, about 2 μg to about 10 μg, about 2 μg to about 9 μg, about 2 μg to about 8 μg, about 2 μg to about 7 μg, about 2 μg to about 6 μg, about 2 μg to about 5 μg, about 3 μg to about 30 μg, about 3 μg to about 29 μg, about 3 μg to about 28 μg, about 3 μg to about 27 μg, about 3 μg to about 26 μg, about 3 μg to about 25 μg, about 3 μg to about 24 μg, about 3 μg to about 23 μg, about 3 μg to about 22 μg, about 3 μg to about 21 μg, about 3 μg to about 20 μg, about 3 μg to about 19 μg, about 3 μg to about 18 μg, about 3 μg to about 17 μg, about 3 μg to about 16 μg, about 3 μg to about 15 μg, about 3 μg to about 14 μg, about 3 μg to about 13 μg, about 3 μg to about 12 μg, about 3 μg to about 11 μg, about 3 μg to about 10 μg, about 3 μg to about 9 μg, about 3 μg to about 8 μg, about 3 μg to about 7 μg, about 3 μg to about 6 μg, about 3 μg to about 5 μg, about 4 μg to about 30 μg, about 4 μg to about 29 μg, about 4 μg to about 28 μg, about 4 μg to about 27 μg, about 4 μg to about 26 μg, about 4 μg to about 25 μg, about 4 μg to about 24 μg, about 4 μg to about 23 μg, about 4 μg to about 22 μg, about 4 μg to about 21 μg, about 4 μg to about 20 μg, about 4 μg to about 19 μg, about 4 μg to about 18 μg, about 4 μg to about 17 μg, about 4 μg to about 16 μg, about 4 μg to about 15 μg, about 4 μg to about 14 μg, about 4 μg to about 13 μg, about 4 μg to about 12 μg, about 4 μg to about 11 μg, about 4 μg to about 10 μg, about 4 μg to about 9 μg, about 4 μg to about 8 μg, about 4 μg to about 7 μg, about 4 μg to about 6 μg, about 4 μg to about 5 μg, about 5 μg to about 30 μg, about 5 μg to about 29 μg, about 5 μg to about 28 μg, about 5 μg to about 27 μg, about 5 μg to about 26 μg, about 5 μg to about 25 μg, about 5 μg to about 24 μg, about 5 μg to about 23 μg, about 5 μg to about 22 μg, about 5 μg to about 21 μg, about 5 μg to about 20 μg, about 5 μg to about 19 μg, about 5 μg to about 18 μg, about 5 μg to about 17 μg, about 5 μg to about 16 μg, about 5 μg to about 15 μg, about 5 μg to about 14 μg, about 5 μg to about 13 μg, about 5 μg to about 12 μg, about 5 μg to about 11 μg, about 5 μg to about 10 μg, about 5 μg to about 9 μg, about 5 μg to about 8 μg, about 5 μg to about 7 μg, about 5 μg to about 6 μg, about 10 μg to about 30 μg, about 10 μg to about 29 μg, about 10 μg to about 28 μg, about 10 μg to about 27 μg, about 10 μg to about 26 μg, about 10 μg to about 25 μg, about 10 μg to about 24 μg, about 10 μg to about 23 μg, about 10 μg to about 22 μg, about 10 μg to about 21 μg, about 10 μg to about 20 μg, about 10 μg to about 19 μg, about 10 μg to about 18 μg, about 10 μg to about 17 μg, about 10 μg to about 16 μg, about 10 μg to about 15 μg, about 10 μg to about 14 μg, about 10 μg to about 13 μg, about 10 μg to about 12 μg, about 10 μg to about 11 μg, about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 29 μg, or about 30 μg of excipient (e.g., alginate, such as sodium alginate). In some embodiments, each dose comprises about 1 μg to about 30 μg of delivery-enhancing agent and/or penetration enhancer and/or surfactant (e.g., alkyl glycoside, such as dodecyl maltoside or DDM), e.g., about 1 μg to about 30 μg, about 1 μg to about 29 μg, about 1 μg to about 28 μg, about 1 μg to about 27 μg, about 1 μg to about 26 μg, about 1 μg to about 25 μg, about 1 μg to about 24 μg, about 1 μg to about 23 μg, about 1 μg to about 22 μg, about 1 μg to about 21 μg, about 1 μg to about 20 μg, about 1 μg to about 19 μg, about 1 μg to about 18 μg, about 1 μg to about 17 μg, about 1 μg to about 16 μg, about 1 μg to about 15 μg, about 1 μg to about 14 μg, about 1 μg to about 13 μg, about 1 μg to about 12 μg, about 1 μg to about 11 μg, about 1 μg to about 10 μg, about 1 μg to about 9 μg, about 1 μg to about 8 μg, about 1 μg to about 7 μg, about 1 μg to about 6 μg, about 1 μg to about 5 μg, about 2 μg to about 30 μg, about 2 μg to about 29 μg, about 2 μg to about 28 μg, about 2 μg to about 27 μg, about 2 μg to about 26 μg, about 2 μg to about 25 μg, about 2 μg to about 24 μg, about 2 μg to about 23 μg, about 2 μg to about 22 μg, about 2 μg to about 21 μg, about 2 μg to about 20 μg, about 2 μg to about 19 μg, about 2 μg to about 18 μg, about 2 μg to about 17 μg, about 2 μg to about 16 μg, about 2 μg to about 15 μg, about 2 μg to about 14 μg, about 2 μg to about 13 μg, about 2 μg to about 12 μg, about 2 μg to about 11 μg, about 2 μg to about 10 μg, about 2 μg to about 9 μg, about 2 μg to about 8 μg, about 2 μg to about 7 μg, about 2 μg to about 6 μg, about 2 μg to about 5 μg, about 3 μg to about 30 μg, about 3 μg to about 29 μg, about 3 μg to about 28 μg, about 3 μg to about 27 μg, about 3 μg to about 26 μg, about 3 μg to about 25 μg, about 3 μg to about 24 μg, about 3 μg to about 23 μg, about 3 μg to about 22 μg, about 3 μg to about 21 μg, about 3 μg to about 20 μg, about 3 μg to about 19 μg, about 3 μg to about 18 μg, about 3 μg to about 17 μg, about 3 μg to about 16 μg, about 3 μg to about 15 μg, about 3 μg to about 14 μg, about 3 μg to about 13 μg, about 3 μg to about 12 μg, about 3 μg to about 11 μg, about 3 μg to about 10 μg, about 3 μg to about 9 μg, about 3 μg to about 8 μg, about 3 μg to about 7 μg, about 3 μg to about 6 μg, about 3 μg to about 5 μg, about 4 μg to about 30 μg, about 4 μg to about 29 μg, about 4 μg to about 28 μg, about 4 μg to about 27 μg, about 4 μg to about 26 μg, about 4 μg to about 25 μg, about 4 μg to about 24 μg, about 4 μg to about 23 μg, about 4 μg to about 22 μg, about 4 μg to about 21 μg, about 4 μg to about 20 μg, about 4 μg to about 19 μg, about 4 μg to about 18 μg, about 4 μg to about 17 μg, about 4 μg to about 16 μg, about 4 μg to about 15 μg, about 4 μg to about 14 μg, about 4 μg to about 13 μg, about 4 μg to about 12 μg, about 4 μg to about 11 μg, about 4 μg to about 10 μg, about 4 μg to about 9 μg, about 4 μg to about 8 μg, about 4 μg to about 7 μg, about 4 μg to about 6 μg, about 4 μg to about 5 μg, about 5 μg to about 30 μg, about 5 μg to about 29 μg, about 5 μg to about 28 μg, about 5 μg to about 27 μg, about 5 μg to about 26 μg, about 5 μg to about 25 μg, about 5 μg to about 24 μg, about 5 μg to about 23 μg, about 5 μg to about 22 μg, about 5 μg to about 21 μg, about 5 μg to about 20 μg, about 5 μg to about 19 μg, about 5 μg to about 18 μg, about 5 μg to about 17 μg, about 5 μg to about 16 μg, about 5 μg to about 15 μg, about 5 μg to about 14 μg, about 5 μg to about 13 μg, about 5 μg to about 12 μg, about 5 μg to about 11 μg, about 5 μg to about 10 μg, about 5 μg to about 9 μg, about 5 μg to about 8 μg, about 5 μg to about 7 μg, about 5 μg to about 6 μg, about 10 μg to about 30 μg, about 10 μg to about 29 μg, about 10 μg to about 28 μg, about 10 μg to about 27 μg, about 10 μg to about 26 μg, about 10 μg to about 25 μg, about 10 μg to about 24 μg, about 10 μg to about 23 μg, about 10 μg to about 22 μg, about 10 μg to about 21 μg, about 10 μg to about 20 μg, about 10 μg to about 19 μg, about 10 μg to about 18 μg, about 10 μg to about 17 μg, about 10 μg to about 16 μg, about 10 μg to about 15 μg, about 10 μg to about 14 μg, about 10 μg to about 13 μg, about 10 μg to about 12 μg, about 10 μg to about 11 μg, about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 29 μg, or about 30 μg of delivery-enhancing agent and/or penetration enhancer and/or surfactant (e.g., alkyl glycoside, such as dodecyl maltoside or DDM.

In some embodiments, the method comprises administering more than one dose of a peptide composition herein. Subsequent doses may have the same amount, less than, or greater than the amount of peptide as the first dose. A subsequent dose may be administered about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the previous dose. In a nonlimiting example a subject may take a dose daily. A subsequent dose may be administered about 1, 2, 3, or 4 weeks after the previous dose. The one or more doses may be about 1, 2, 3, 4, 5, 6, 7, 8, 9, 10, 11, 12, 13, 14, 15, 16, 17, 18, 19, or 20 doses. In a non-limiting example, the composition is administered in about 5 daily doses.

In some embodiments, the method comprises administering to a subject the first dose of the composition at a first time point and a second dose of the compound at a second time point. In some cases, the second time point is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the first time point. In some cases, the second time point is about 1, 2, 3, or 4 weeks after the first time point. In some cases, the second dose comprises the same amount of composition as the first dose. In some cases, the second dose comprises a different amount of composition as the first dose. In some cases, the second dose comprises about 0.1 mg to about 8 mg of the composition. In example embodiments, the second dose comprises the same dose as the first dose of the composition, administered the day after the first dose.

In some embodiments, the method comprises administering to a subject a third dose of the composition at a third time point. In some cases, the third time point is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the second time point. In some cases, the third time point is about 1, 2, 3, or 4 weeks after the second time point. In some cases, the third dose comprises the same amount of composition as the second dose. In some cases, the third dose comprises a different amount of composition as the second dose. In some cases, the third dose comprises about 0.1 mg to about 8 mg of the composition. In example embodiments, the third dose comprises the same dose as the second dose of the composition, administered the day after the second dose.

In some embodiments, the method comprises administering to a subject a fourth dose of the composition at a fourth time point. In some cases, the fourth time point is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the third time point. In some cases, the fourth time point is about 1, 2, 3, or 4 weeks after the third time point. In some cases, the fourth dose comprises the same amount of composition as the third dose. In some cases, the fourth dose comprises a different amount of composition as the third dose. In some cases, the fourth dose comprises about 0.1 mg to about 8 mg of the composition. In example embodiments, the fourth dose comprises the same dose as the third dose of the composition, administered the day after the third dose.

In some embodiments, the method comprises administering to a subject a fifth dose of the composition at a fifth time point. In some cases, the fifth time point is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the fourth time point. In some cases, the fifth time point is about 1, 2, 3, or 4 weeks after the fourth time point. In some cases, the fifth dose comprises the same amount of composition as the fourth dose. In some cases, the fifth dose comprises a different amount of composition as the fourth dose. In some cases, the fifth dose comprises about 0.1 mg to about 8 mg of the composition. In example embodiments, the fifth dose comprises the same dose as the fourth dose of the composition, administered the day after the fourth dose.

In one aspect, a method of treatment comprises administrating to the subject a first dose on day 1, a second dose on day 2, a third dose on day 3, a fourth dose on day 4, a fifth dose on day 5, no dose on day 6, and no dose on day 7. In some embodiments this regimen is optionally repeated as necessary, e.g., every week. For instance, on day 8, the first dose is taken, on day 9 the second dose is taken, and so on.

### EXAMPLES

The following examples are illustrative of the embodiments described herein and are not to be interpreted as limiting the scope of this disclosure. To the extent that specific materials are mentioned, it is merely for purposes of illustration and is not intended to be limiting. One skilled in the art may develop equivalent means or reactants without the exercise of inventive capacity and without departing from the scope of this disclosure.

### Example 1: Example GHRH Peptide Composition

In one embodiment a GHRH peptide is formulated in a composition for oral administration. The active ingredients of the composition are as shown in Table 2 below. There is a GHRH peptide component of Sermorelin as SEQ ID NO: 2. Sermorelin (as SEQ ID NO: 2) comprises 1 mg of the 1.25 mg tablet composition, or 80%. The delivery-enhancing agent of the composition is glycine. Glycine comprises 0.25 mg of the 1.25 mg tablet composition, or 20%.

TABLE 2
Example Peptide Composition

| | | |
|---|---|---|
| Sermorelin (as SEQ ID NO: 2) | 1,000 | mcg |
| Glycine | 250 | mcg |
| Total | 1,250 | mcg |
| | 1.25 | mg |
| | 0.00125 | g |

In another embodiment a GHRH peptide is formulated in a composition for oral administration. The active ingredients of the composition are as shown in Table 3 below. There is a GHRH peptide component of Sermorelin as SEQ ID NO: 2. Sermorelin (as SEQ ID NO: 2) comprises 1 mg of the 2 mg tablet composition, or 50%. The delivery-enhancing agent of the composition is glycine. Glycine comprises 1 mg of the 2 mg tablet composition, or 50%.

TABLE 3
Example Peptide Composition

| | | |
|---|---|---|
| Sermorelin (as SEQ ID NO: 2) | 1,000 | mcg |
| Glycine | 1,000 | mcg |
| Total | 2,000 | mcg |
| | 2 | mg |
| | 0.002 | g |

In another embodiment a GHRH peptide is formulated in a composition for oral administration. The active ingredients of the composition are as shown in Table 4 below. There is a GHRH peptide component of Sermorelin as SEQ ID NO: 2. Sermorelin (as SEQ ID NO: 2) comprises 1 mg of the 2.5 mg tablet composition, or 40%. The delivery-enhancing agent of the composition is glycine. Glycine comprises 1.5 mg of the 2.5 mg tablet composition, or 60%.

TABLE 4
Example Peptide Composition

| | | |
|---|---|---|
| Sermorelin (as SEQ ID NO: 2) | 1,000 | mcg |
| Glycine | 1,500 | mcg |
| Total | 2,500 | mcg |
| | 2.5 | mg |
| | 0.0025 | g |

In another embodiment a GHRH peptide is formulated in a composition for oral administration. The active ingredients of the composition are as shown in Table 5 below. There is a GHRH peptide component of Sermorelin as SEQ ID NO: 2. Sermorelin (as SEQ ID NO: 2) comprises 1 mg of the 3 mg tablet composition, or about 33%. The delivery-enhancing agent of the composition is glycine. Glycine comprises 2 mg of the 3 mg tablet composition, or about 67%.

TABLE 5
Example Peptide Composition

| | | |
|---|---|---|
| Sermorelin (as SEQ ID NO: 2) | 1,000 | mcg |
| Glycine | 2,000 | mcg |
| Total | 3,000 | mcg |
| | 3 | mg |
| | 0.003 | g |

In another embodiment a GHRH peptide is formulated in a composition for oral administration. The active ingredients of the composition are as shown in Table 6 below. There is a GHRH peptide component of Sermorelin as SEQ ID NO: 2. Sermorelin (as SEQ ID NO: 2) comprises 1 mg of the 3.5 mg tablet composition, or 28.6%. The delivery-enhancing agent of the composition is glycine. Glycine comprises 2.5 mg of the 3.5 mg tablet composition, or 71.4%.

TABLE 6
Example Peptide Composition

| | | |
|---|---|---|
| Sermorelin (as SEQ ID NO: 2) | 1,000 | mcg |
| Glycine | 2,500 | mcg |
| Total | 3,500 | mcg |
| | 3.5 | mg |
| | 0.0035 | g |

In another embodiment a GHRH peptide is formulated in a composition for oral administration. The active ingredients of the composition are as shown in Table 7 below. There is a GHRH peptide component of Sermorelin as SEQ ID NO: 2. Sermorelin (as SEQ ID NO: 2) comprises 1 mg of the 4 mg tablet composition, or 25%. The delivery-enhancing agent of the composition is glycine. Glycine comprises 3 mg of the 4 mg tablet composition, or 75%.

TABLE 7
Example Peptide Composition

| | | |
|---|---|---|
| Sermorelin (as SEQ ID NO: 2) | 1,000 | mcg |
| Glycine | 3,000 | mcg |
| Total | 4,000 | mcg |
| | 4 | mg |
| | 0.004 | g |

### Example 2: Manufacture of a GHRH Peptide Formulation

The GHRH peptide formulation of Example 1 is manufactured into an oral dissolving tablet, e.g., using lyophilization as shown in FIG. 2.

### Example 3: Method of Use

A person suffers from insomnia; they are incapable of having deep, restful sleep. They take the tablet of Example 2 (e.g., the 1.25 mg, 2 mg, 2.5 mg, 3 mg, 3.5 mg, or 4 mg tablet) daily for five consecutive days. Afterward, the person does not take the tablet for two days. This regimen of five days on, two days off is repeated weekly as necessary to continue the therapeutic effects of the peptide formulation.

The foregoing description of various embodiments known to the applicant at this time of filing the application has been presented and is intended for the purposes of illustration and description. The present description is not intended to be exhaustive nor limited to the precise form disclosed and many modifications and variations are possible in the light of the above teachings. The embodiments described serve to explain principles and practical applications, and to enable others skilled in the art to utilize the various embodiments, optionally with various modifications, as are suited to the particular use contemplated. Therefore, it is intended that the disclosure not be limited to the particular embodiments disclosed.

What is claimed is:

1. A composition comprising a growth hormone-releasing hormone (GHRH) peptide and glycine.

2. The composition of claim 1, wherein about 50 μg to about 3000 μg of glycine is present in the composition.

3. The composition of claim 1, comprising a delivery-enhancing agent.

4. A composition comprising a growth hormone-releasing hormone (GHRH) peptide and a delivery-enhancing agent.

5. The composition of claim 3 or claim 4, wherein the delivery-enhancing agent comprises an alkyl glycoside.

6. The composition of claim 5, wherein the alkyl glycoside comprises n-dodecyl-β-D-maltoside.

7. The composition of claim 3 or claim 4, wherein the delivery-enhancing agent comprises a membrane penetration-enhancing agent.

8. The composition of claim 3 or claim 4, wherein the delivery-enhancing agent comprises (i) a surfactant, (ii) a bile salt, (ii) a phospholipid additive, mixed micelle, liposome, or carrier, (iii) an alcohol, (iv) an enamine, (v) an NO donor compound, (vi) a long-chain amphipathic molecule, (vii) a small hydrophobic penetration enhancer, (viii) sodium or a salicylic acid derivative, (ix) a glycerol ester of acetoacetic acid, (x) a cyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, (x) an inhibitor of cholesterol synthesis, (xi) glycine, and (xii) any combination of the delivery-enhancing agents recited in (i)-(xi).

9. The composition of claim 3 or claim 4, wherein about 1 μg to about 30 μg of the delivery-enhancing agent is present in the composition.

10. The composition of claim 1, further comprising a stimulatory agent.

11. A composition comprising a growth hormone-releasing hormone (GHRH) peptide and a stimulatory agent.

12. The composition of claim 10 or claim 11, wherein the stimulatory agent induces secretion of human growth hormone.

13. The composition of claim 10 or claim 11, wherein about 50 μg to about 3000 μg of the stimulatory agent is present in the composition.

14. The composition of claim 1, further comprising a steroid.

15. A composition comprising a growth hormone-releasing hormone (GHRH) peptide and a steroid.

16. The composition of claim 14 or claim 15, wherein the steroid comprises ginsenoside Rg3.

17. The composition of claim 14 or claim 15, wherein about 200 μg to about 2000 μg of the steroid is present in the composition.

18. The composition of claim 1, further comprising a vitamin.

19. A composition comprising a growth hormone-releasing hormone (GHRH) peptide and a vitamin.

20. The composition of claim 18 or claim 19, wherein the vitamin comprises nicotinamide riboside.

21. The composition of claim 18 or claim 19, wherein the vitamin comprises methylcobalamin.

22. The composition of claim 18 or claim 19, wherein about 200 μg to about 2000 μg of the vitamin is present in the composition.

23. The composition of claim 1, further comprising an excipient.

24. A composition comprising a growth hormone-releasing hormone (GHRH) peptide and an excipient.

25. The composition of claim 23 or claim 24, wherein the excipient is a penetration enhancer.

26. The composition of claim 23 or claim 24, wherein the excipient comprises sodium alginate.

27. The composition of claim 23 or claim 24, wherein about 1 μg to about 30 μg of the excipient is present in the composition.

28. The composition of claim 1, wherein the GHRH peptide comprises amino acids 1-29 of human GHRH.

29. The composition of claim 1, wherein the GHRH peptide comprises a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1 (YADAIFTNSY RKVLGQLSAR KLLQDIMSR) or SEQ ID NO: 2 (YADAIFTNSY RKVLGQLSAR KLLQDIMSR-NH2).

30. The composition of claim 1, wherein about 200 μg to about 2000 μg of the GHRH peptide is present in the composition.

31. The composition of claim 1, formulated in an oral dissolving tablet.

32. The composition of claim 31, wherein the composition disintegrates in water in less than about 30 seconds.

33. The composition of claim 1, wherein the composition is prepared in a process comprising lyophilization.

34. A method of preparing a tablet comprising the composition of claim 1, the method comprising lyophilizing a formulation comprising the composition of claim 1.

35. A method of improving the health of a subject, the method comprising administering to the subject a composition of claim 1.

36. The method of claim 35, wherein the composition increases testosterone, improves endurance, improves performance, boosts the immune system, boosts metabolism, naturally modulates hGH, improves libido, improves cognitive function, improves mood, increases energy, improves skin texture & tone, repairs DNA and maintains cell health, increases strength, helps to build lean muscles, improves muscle mass, improves skin health, improves bone health, has a calming effect, has a deep sleep effect, reduces time it takes to fall asleep, or boosts daytime alertness, or any combination of two or more thereof, as compared to the subject prior to administration of the composition.
